SEQUENCE LISTING (249 sequences)

SEQ ID NO: 1 - protein, 8 aa, Artificial Sequence
  Synthetic sequence
RRRRRRRR

SEQ ID NO: 2 - protein, 25 aa, Artificial Sequence
  Synthetic construct
GLFHAIAHFIHGGWHGLIHGWYGGC

SEQ ID NO: 3 - protein, 30 aa, Artificial Sequence
  Synthetic construct
WEARLARALARALARHLARALARALRAGEA

SEQ ID NO: 4 - protein, 30 aa, Artificial Sequence
  Synthetic construct
WEAKLAKALAKALAKHLAKALAKALKAGEA

SEQ ID NO: 5 - protein, 30 aa, Artificial Sequence
  Synthetic construct
WEAALAEALAEALAEHLAEALAEALEALAA

SEQ ID NO: 6 - protein, 23 aa, Artificial Sequence
  Synthetic construct
GLFEAIEGFIENGWEGMIDGWYG

SEQ ID NO: 7-8 - skipped (2 entries)

SEQ ID NO: 9 - protein, 42 aa, Artificial Sequence
  Synthetic construct
GNQSSNFGPMKGGNFGGRSSGPYGGGGQYFAKPRNQGGYGGC

SEQ ID NO: 10 - protein, 7 aa, Artificial Sequence
  Synthetic construct
RRMKWKK

SEQ ID NO: 11 - protein, 7 aa, Artificial Sequence
  Synthetic construct
PKKKRKV

SEQ ID NO: 12 - protein, 16 aa, Artificial Sequence
  Synthetic construct
KRPAATKKAGQAKKKK

SEQ ID NO: 13-19 - skipped (7 entries)

SEQ ID NO: 20 - DNA, 36 bp, Artificial Sequence
  Synthetic construct
guuuuagagcuaugcuguuuugaauggucccaaaac

SEQ ID NO: 21 - DNA, 36 bp, Artificial Sequence
  Synthetic construct
guuuuagagcuauguuauuuugaaugcuaacaaaac

SEQ ID NO: 22 - DNA, 36 bp, Artificial Sequence
  Synthetic construct
guuuuagagcuguguuguuucgaaugguuccaaaac

SEQ ID NO: 23 - DNA, 36 bp, Artificial Sequence
  Synthetic construct
guuuuuguacucucaagauuuaaguaacuguacaac

SEQ ID NO: 24 - DNA, 37 bp, Artificial Sequence
  Synthetic construct
cuaacaguaguuuaccaaauaauucagcaacugaaac

SEQ ID NO: 25 - DNA, 37 bp, Artificial Sequence
  Synthetic construct
gcaacacuuuauagcaaauccgcuuagccugugaaac

SEQ ID NO: 26 - DNA, 38 bp, Artificial Sequence
  Synthetic construct
  misc_feature (1)..(10): where n at each of positions 1-10 can be present or absent and n is a, c, t, g, u, or modified forms thereof
  misc_feature (12)..(12): where n at position 12 can be present or absent and n is a, c, t, g, u, or modified forms thereof
  misc_feature (14)..(35): where n at each of positions 14-35 can be present or absent and n is a, c, t, g, u, or modified forms thereof
nnnnnnnnnnununnnnnnnnnnnnnnnnnnnnnnaac

SEQ ID NO: 27 - DNA, 12 bp, Artificial Sequence
  Synthetic construct
  misc_feature (1)..(10): where n at each of positions 1-10 can be present or absent and n is a, c, t, g, u, or modified forms thereof
  misc_feature (12)..(12): where n at position 12 can be present or absent and n is a, c, t, g, u, or modified forms thereof
nnnnnnnnnnun

SEQ ID NO: 28 - DNA, 16 bp, Artificial Sequence
  Synthetic construct
  misc_feature (1)..(10): where n at each of positions 1-10 can be present or absent and n is a, c, t, g, u, or modified forms thereof
  misc_feature (12)..(12): where n at position 12 can be present or absent and n is a, c, t, g, u, or modified forms thereof
  misc_feature (14)..(16): where n at each of positions 14-16 can be present or absent and n is a, c, t, g, u, or modified forms thereof
nnnnnnnnnnununnn

SEQ ID NO: 29 - DNA, 36 bp, Artificial Sequence
  Synthetic construct
  misc_feature (6)..(6): where n at position 6 can be present or absent and n is a, c, t, g, u, or modified forms thereof
  misc_feature (8)..(9): where n at each of positions 8-9 can be present or absent and n is a, c, t, g, u, or modified forms thereof
  misc_feature (12)..(12): where n at position 12 can be present or absent and n is a, c, t, g, u, or modified forms thereof
  misc_feature (14)..(18): where n at each of positions 14-18 can be present or absent and n is a, c, t, g, u, or modified forms thereof
  misc_feature (21)..(22): where n at each of positions 21-22 can be present or absent and n is a, c, t, g, u, or modified forms thereof
  misc_feature (24)..(24): where n at position 24 can be present or absent and n is a, c, t, g, u, or modified forms thereof
  misc_feature (26)..(31): where n at each of positions 26-31 can be present or absent and n is a, c, t, g, u, or modified forms thereof
  misc_feature (33)..(33): where n at position 33 can be present or absent and n is a, c, t, g, u, or modified forms thereof
guuuungnncununnnnnuunnanunnnnnnanaac

SEQ ID NO: 30 - DNA, 12 bp, Artificial Sequence
  Synthetic construct
  misc_feature (6)..(6): where n at position 6 can be present or absent and n is a, c, t, g, u, or modified forms thereof
  misc_feature (8)..(9): where n at each of positions 8-9 can be present or absent and n is a, c, t, g, u, or modified forms thereof
  misc_feature (12)..(12): where n at position 12 can be present or absent and n is a, c, t, g, u, or modified forms thereof
guuuungnncun

SEQ ID NO: 31 - DNA, 38 bp, Artificial Sequence
  Synthetic construct
  misc_feature (1)..(2): where n at each of positions 1-2 can be present or absent and n is a, c, t, g, u, or modified forms thereof
  misc_feature (7)..(7): where n at position 7 can be present or absent and n is a, c, t, g, u, or modified forms thereof
  misc_feature (9)..(10): where n at each of positions 9-10 can be present or absent and n is a, c, t, g, u, or modified forms thereof
  misc_feature (12)..(12): where n at position 12 can be present or absent and n is a, c, t, g, u, or modified forms thereof
  misc_feature (15)..(15): where n at position 15 can be present or absent and n is a, c, t, g, u, or modified forms thereof
  misc_feature (21)..(24): where n at each of positions 21-24 can be present or absent and n is a, c, t, g, u, or modified forms thereof
  misc_feature (26)..(26): where n at position 26 can be present or absent and n is a, c, t, g, u, or modified forms thereof
  misc_feature (28)..(28): where n at position 28 can be present or absent and n is a, c, t, g, u, or modified forms thereof
  misc_feature (30)..(32): where n at each of positions 30-32 can be present or absent and n is a, c, t, g, u, or modified forms thereof
nnaacanunnunuancaaaunnnnunancnnnugaaac

SEQ ID NO: 32 - DNA, 16 bp, Artificial Sequence
  Synthetic construct
  misc_feature (1)..(2): where n at each of positions 1-2 can be present or absent and n is a, c, t, g, u, or modified forms thereof
  misc_feature (7)..(7): where n at position 7 can be present or absent and n is a, c, t, g, u, or modified forms thereof
  misc_feature (9)..(10): where n at each of positions 9-10 can be present or absent and n is a, c, t, g, u, or modified forms thereof
  misc_feature (12)..(12): where n at position 12 can be present or absent and n is a, c, t, g, u, or modified forms thereof
  misc_feature (15)..(15): where n at position 12 can be present or absent and n is a, c, t, g, u, or modified forms thereof
  misc_feature (15)..(15): where n at position 15 can be present or absent and n is a, c, t, g, u, or modified forms thereof
nnaacanunnunuanc

SEQ ID NO: 33-39 - skipped (7 entries)

SEQ ID NO: 40 - DNA, 36 bp, Artificial Sequence
  Synthetic construct
uuguuggaaccauucaaaacagcauagcaaguuaaa

SEQ ID NO: 41 - DNA, 36 bp, Artificial Sequence
  Synthetic construct
auauuguuaguauucaaaauaacauagcaaguuaaa

SEQ ID NO: 42 - DNA, 36 bp, Artificial Sequence
  Synthetic construct
gguuugaaaccauucgaaacaacacagcgaguuaaa

SEQ ID NO: 43 - DNA, 36 bp, Artificial Sequence
  Synthetic construct
cuuacacaguuacuuaaaucuugcagaagcuacaaa

SEQ ID NO: 44 - DNA, 37 bp, Artificial Sequence
  Synthetic construct
guuucaguuguuagauuauuugguauguacuuguguu

SEQ ID NO: 45 - DNA, 37 bp, Artificial Sequence
  Synthetic construct
auuacagagcauuaauuauuugguacauuuauaauuu

SEQ ID NO: 46 - DNA, 37 bp, Artificial Sequence
  Synthetic construct
uuucaaggcaucgaacggauuugcuauaaaguguugc

SEQ ID NO: 47 - DNA, 37 bp, Artificial Sequence
  Synthetic construct
uuuguuaaagcuggaugggauuauuauagaguguugc

SEQ ID NO: 48 - DNA, 41 bp, Artificial Sequence
  Synthetic construct
  misc_feature (1)..(21): where n at each of positions 1-21 can be present or absent and n is a, c, t, g, u, or modified forms thereof
  misc_feature (23)..(28): where n at each of positions 23-28 can be present or absent and n is a, c, t, g, u, or modified forms thereof
  misc_feature (30)..(41): where n at each of positions 30-41 can be present or absent and n is a, c, t, g, u, or modified forms thereof
nnnnnnnnnnnnnnnnnnnnnannnnnnannnnnnnnnnnn

SEQ ID NO: 49 - DNA, 14 bp, Artificial Sequence
  Synthetic construct
  misc_feature (1)..(1): where n at position 1 can be present or absent and n is a, c, t, g, u, or modified forms thereof
  misc_feature (3)..(14): where n at each of positions 3-14 can be present or absent and n is a, c, t, g, u, or modified forms thereof
nannnnnnnnnnnn

SEQ ID NO: 50 - DNA, 12 bp, Artificial Sequence
  Synthetic construct
  misc_feature (1)..(12): where n at each of positions 1-12 can be present or absent and n is a, c, t, g, u, or modified forms thereof
nnnnnnnnnnnn

SEQ ID NO: 51 - DNA, 37 bp, Artificial Sequence
  Synthetic construct
  misc_feature (1)..(11): where n at each of positions 1-11 can be present or absent and n is a, c, t, g, u, or modified forms thereof
  misc_feature (13)..(13): where n at position 13 can be present or absent and n is a, c, t, g, u, or modified forms thereof
  misc_feature (15)..(16): where n at each of positions 15-16 can be present or absent and n is a, c, t, g, u, or modified forms thereof
  misc_feature (19)..(25): where n at each of positions 19-25 can be present or absent and n is a, c, t, g, u, or modified forms thereof
  misc_feature (28)..(31): where n at each of positions 28-31 can be present or absent and n is a, c, t, g, u, or modified forms thereof
  misc_feature (33)..(34): where n at each of positions 33-34 can be present or absent and n is a, c, t, g, u, or modified forms thereof
nnnnnnnnnnnanunnaannnnnnnagnnnnunnaaa

SEQ ID NO: 52 - DNA, 13 bp, Artificial Sequence
  Synthetic construct
  misc_feature (1)..(1): where n at position 1 can be present or absent and n is a, c, t, g, u, or modified forms thereof
  misc_feature (4)..(7): where n at each of positions 4-7 can be present or absent and n is a, c, t, g, u, or modified forms thereof
  misc_feature (9)..(10): where n at each of positions 9-10 can be present or absent and n is a, c, t, g, u, or modified forms thereof
nagnnnnunnaaa

SEQ ID NO: 53 - DNA, 39 bp, Artificial Sequence
  Synthetic construct
  misc_feature (1)..(2): where n at each of positions 1-2 can be present or absent and n is a, c, t, g, u, or modified forms thereof
  misc_feature (4)..(11): where n at each of positions 4-11 can be present or absent and n is a, c, t, g, u, or modified forms thereof
  misc_feature (13)..(15): where n at each of positions 13-15 can be present or absent and n is a, c, t, g, u, or modified forms thereof
  misc_feature (17)..(21): where n at each of positions 17-21 can be present or absent and n is a, c, t, g, u, or modified forms thereof
  misc_feature (24)..(25): where n at each of positions 24-25 can be present or absent and n is a, c, t, g, u, or modified forms thereof
  misc_feature (28)..(33): where n at each of positions 28-33 can be present or absent and n is a, c, t, g, u, or modified forms thereof
  misc_feature (35)..(39): where n at each of positions 35-39 can be present or absent and n is a, c, t, g, u, or modified forms thereof
nnunnnnnnnnunnnannnnnuunnuannnnnnunnnnn

SEQ ID NO: 54 - DNA, 16 bp, Artificial Sequence
  Synthetic construct
  misc_feature (1)..(2): where n at each of positions 1-2 can be present or absent and n is a, c, t, g, u, or modified forms thereof
  misc_feature (5)..(10): where n at each of positions 5-10 can be present or absent and n is a, c, t, g, u, or modified forms thereof
  misc_feature (12)..(16): where n at each of positions 12-16 can be present or absent and n is a, c, t, g, u, or modified forms thereof
nnuannnnnnunnnnn

SEQ ID NO: 55-59 - skipped (5 entries)

SEQ ID NO: 60 - DNA, 88 bp, Artificial Sequence
  Synthetic construct
guuggaaccauucaaaacagcauagcaaguuaaaauaaggcuaguccguuaucaacuugaaaaaguggcaccgagucggugcuuuuuu

SEQ ID NO: 61 - DNA, 93 bp, Artificial Sequence
  Synthetic construct
auauuguuaguauucaaaauaacauagcaaguuaaaauaaggcuuuguccguuaucaacuuuuaauuaaguagcgcuguuucggcgcuuuuuu

SEQ ID NO: 62 - DNA, 95 bp, Artificial Sequence
  Synthetic construct
uugugguuugaaaccauucgaaacaacacagcgaguuaaaauaaggcuuaguccguacucaacuugaaaagguggcaccgauucgguguuuuuuu

SEQ ID NO: 63 - DNA, 118 bp, Artificial Sequence
  Synthetic construct
uaauaauaguguaagggacgccuuacacaguuacuuaaaucuugcagaagcuacaaagauaaggcuucaugccgaaaucaacacccugucauuuuauggcaggguguuuucguuauuu

SEQ ID NO: 64 - DNA, 121 bp, Artificial Sequence
  Synthetic construct
  misc_feature (1)..(6): where n at each of positions 1-6 can be present or absent and n is a, c, t, g, u, or modified forms thereof
  misc_feature (8)..(11): where n at each of positions 8-11 can be present or absent and n is a, c, t, g, u, or modified forms thereof
  misc_feature (14)..(15): where n at each of positions 14-15 can be present or absent and n is a, c, t, g, u, or modified forms thereof
  misc_feature (17)..(25): where n at each of positions 17-25 can be present or absent and n is a, c, t, g, u, or modified forms thereof
  misc_feature (27)..(27): where n at position 27 can be present or absent and n is a, c, t, g, u, or modified forms thereof
  misc_feature (29)..(30): where n at each of positions 29-30 can be present or absent and n is a, c, t, g, u, or modified forms thereof
  misc_feature (32)..(35): where n at each of positions 32-35 can be present or absent and n is a, c, t, g, u, or modified forms thereof
  misc_feature (37)..(45): where n at each of positions 37-45 can be present or absent and n is a, c, t, g, u, or modified forms thereof
  misc_feature (48)..(50): where n at each of positions 48-50 can be present or absent and n is a, c, t, g, u, or modified forms thereof
  misc_feature (53)..(53): where n at position 53 can be present or absent and n is a, c, t, g, u, or modified forms thereof
  misc_feature (56)..(58): where n at each of positions 56-58 can be present or absent and n is a, c, t, g, u, or modified forms thereof
  misc_feature (60)..(60): where n at position 60 can be present or absent and n is a, c, t, g, u, or modified forms thereof
  misc_feature (69)..(71): where n at each of positions 69-71 can be present or absent and n is a, c, t, g, u, or modified forms thereof
  misc_feature (73)..(73): where n at position 73 can be present or absent and n is a, c, t, g, u, or modified forms thereof
  misc_feature (77)..(79): where n at each of positions 77-79 can be present or absent and n is a, c, t, g, u, or modified forms thereof
  misc_feature (85)..(88): where n at each of positions 85-88 can be present or absent and n is a, c, t, g, u, or modified forms thereof
  misc_feature (90)..(92): where n at each of positions 90-92 can be present or absent and n is a, c, t, g, u, or modified forms thereof
  misc_feature (94)..(98): where n at each of positions 94-98 can be present or absent and n is a, c, t, g, u, or modified forms thereof
  misc_feature (100)..(100): where n at position 100 can be present or absent and n is a, c, t, g, u, or modified forms thereof
  misc_feature (103)..(105): where n at each of positions 103-105 can be present or absent and n is a, c, t, g, u, or modified forms thereof
  misc_feature (107)..(108): where n at each of positions 107-108 can be present or absent and n is a, c, t, g, u, or modified forms thereof
  misc_feature (110)..(113): where n at each of positions 110-113 can be present or absent and n is a, c, t, g, u, or modified forms thereof
  misc_feature (115)..(115): where n at position 115 can be present or absent and n is a, c, t, g, u, or modified forms thereof
  misc_feature (118)..(118): where n at position 118 can be present or absent and n is a, c, t, g, u, or modified forms thereof
nnnnnnunnnnugnnannnnnnnnnuncnnannnncnnnnnnnnngcnnnagnuannnanauaaggcunnnunccgnnnucaacnnnnunnnannnnnungcnnngnnunnnngnuunuuu

SEQ ID NO: 65 - DNA, 39 bp, Artificial Sequence
  Synthetic construct
  misc_feature (1)..(8): where n at each of positions 1-8 can be present or absent and n is a, c, t, g, u, or modified forms thereof
  misc_feature (11)..(13): where n at each of positions 11-13 can be present or absent and n is a, c, t, g, u, or modified forms thereof
  misc_feature (16)..(16): where n at position 16 can be present or absent and n is a, c, t, g, u, or modified forms thereof
  misc_feature (19)..(21): where n at each of positions 19-21 can be present or absent and n is a, c, t, g, u, or modified forms thereof
  misc_feature (23)..(23): where n at position 23 can be present or absent and n is a, c, t, g, u, or modified forms thereof
  misc_feature (32)..(34): where n at each of positions 32-34 can be present or absent and n is a, c, t, g, u, or modified forms thereof
  misc_feature (36)..(36): where n at position 36 can be present or absent and n is a, c, t, g, u, or modified forms thereof
nnnnnnnngcnnnagnuannnanauaaggcunnnunccg

SEQ ID NO: 66-69 - skipped (4 entries)

SEQ ID NO: 70 - DNA, 12 bp, Artificial Sequence
  Synthetic construct
guuuuagagcua

SEQ ID NO: 71 - DNA, 26 bp, Artificial Sequence
  Synthetic construct
uagcaaguuaaaauaaggcuaguccg

SEQ ID NO: 72-79 - skipped (8 entries)

SEQ ID NO: 80 - DNA, 39 bp, Artificial Sequence
  Synthetic construct
  misc_feature (13)..(13): where n is any useful linker
guuuuagagcuanuagcaaguuaaaauaaggcuaguccg

SEQ ID NO: 81 - DNA, 80 bp, Artificial Sequence
  Synthetic construct
  misc_feature (1)..(10): where n at each of positions 1-10 can be present or absent and n is a, c, t, g, u, or modified forms thereof
  misc_feature (12)..(12): where n at position 12 can be present or absent and n is a, c, t, g, u, or modified forms thereof
  misc_feature (14)..(35): where n at each of positions 14-35 can be present or absent and n is a, c, t, g, u, or modified forms thereof
  misc_feature (39)..(39): where n is any useful linker
  misc_feature (40)..(60): where n at each of positions 40-60 can be present or absent and n is a, c, t, g, u, or modified forms thereof
  misc_feature (62)..(67): where n at each of positions 62-67 can be present or absent and n is a, c, t, g, u, or modified forms thereof
  misc_feature (69)..(80): where n at each of positions 69-80 can be present or absent and n is a, c, t, g, u, or modified forms thereof
nnnnnnnnnnununnnnnnnnnnnnnnnnnnnnnnaacnnnnnnnnnnnnnnnnnnnnnnannnnnnannnnnnnnnnnn

SEQ ID NO: 82 - DNA, 27 bp, Artificial Sequence
  Synthetic construct
  misc_feature (1)..(10): where n at each of positions 1-10 can be present or absent and n is a, c, t, g, u, or modified forms thereof
  misc_feature (12)..(12): where n at position 12 can be present or absent and n is a, c, t, g, u, or modified forms thereof
  misc_feature (13)..(13): where n is any useful linker
  misc_feature (14)..(14): where n at position 14 can be present or absent and n is a, c, t, g, u, or modified forms thereof
  misc_feature (16)..(27): where n at each of positions 16-27 can be present or absent and n is a, c, t, g, u, or modified forms thereof
nnnnnnnnnnunnnannnnnnnnnnnn

SEQ ID NO: 83 - DNA, 31 bp, Artificial Sequence
  Synthetic construct
  misc_feature (1)..(10): where n at each of positions 1-10 can be present or absent and n is a, c, t, g, u, or modified forms thereof
  misc_feature (12)..(12): where n at position 12 can be present or absent and n is a, c, t, g, u, or modified forms thereof
  misc_feature (14)..(16): where n at each of positions 14-16 can be present or absent and n is a, c, t, g, u, or modified forms thereof
  misc_feature (17)..(17): where n is any useful linker
  misc_feature (18)..(18): where n at position 18 can be present or absent and n is a, c, t, g, u, or modified forms thereof
  misc_feature (20)..(31): where n at each of positions 20-31 can be present or absent and n is a, c, t, g, u, or modified forms thereof
nnnnnnnnnnununnnnnannnnnnnnnnnn

SEQ ID NO: 84 - DNA, 52 bp, Artificial Sequence
  Synthetic construct
  misc_feature (1)..(10): where n at each of positions 1-10 can be present or absent and n is a, c, t, g, u, or modified forms thereof
  misc_feature (12)..(12): where n at position 12 can be present or absent and n is a, c, t, g, u, or modified forms thereof
  misc_feature (13)..(13): where n is any useful linker
  misc_feature (14)..(21): where n at each of positions 14-21 can be present or absent and n is a, c, t, g, u, or modified forms thereof
  misc_feature (24)..(26): where n at each of positions 24-26 can be present or absent and n is a, c, t, g, u, or modified forms thereof
  misc_feature (29)..(29): where n at position 29 can be present or absent and n is a, c, t, g, u, or modified forms thereof
  misc_feature (32)..(34): where n at each of positions 32-34 can be present or absent and n is a, c, t, g, u, or modified forms thereof
  misc_feature (36)..(36): where n at position 36 can be present or absent and n is a, c, t, g, u, or modified forms thereof
  misc_feature (45)..(47): where n at each of positions 45-47 can be present or absent and n is a, c, t, g, u, or modified forms thereof
  misc_feature (49)..(49): where n at position 49 can be present or absent and n is a, c, t, g, u, or modified forms thereof
nnnnnnnnnnunnnnnnnnnngcnnnagnuannnanauaaggcunnnunccg

SEQ ID NO: 85 - DNA, 56 bp, Artificial Sequence
  Synthetic construct
  misc_feature (1)..(10): where n at each of positions 1-10 can be present or absent and n is a, c, t, g, u, or modified forms thereof
  misc_feature (12)..(12): where n at position 12 can be present or absent and n is a, c, t, g, u, or modified forms thereof
  misc_feature (14)..(16): where n at each of positions 14-16 can be present or absent and n is a, c, t, g, u, or modified forms thereof
  misc_feature (17)..(17): where n is any useful linker
  misc_feature (18)..(25): where n at each of positions 18-25 can be present or absent and n is a, c, t, g, u, or modified forms thereof
  misc_feature (28)..(30): where n at each of positions 28-30 can be present or absent and n is a, c, t, g, u, or modified forms thereof
  misc_feature (33)..(33): where n at position 33 can be present or absent and n is a, c, t, g, u, or modified forms thereof
  misc_feature (36)..(38): where n at each of positions 36-38 can be present or absent and n is a, c, t, g, u, or modified forms thereof
  misc_feature (40)..(40): where n at position 40 can be present or absent and n is a, c, t, g, u, or modified forms thereof
  misc_feature (49)..(51): where n at each of positions 49-51 can be present or absent and n is a, c, t, g, u, or modified forms thereof
  misc_feature (53)..(53): where n at position 53 can be present or absent and n is a, c, t, g, u, or modified forms thereof
nnnnnnnnnnununnnnnnnnnnnngcnnnagnuannnanauaaggcunnnunccg

SEQ ID NO: 86 - DNA, 74 bp, Artificial Sequence
  Synthetic construct
  misc_feature (6)..(6): where n at position 6 can be present or absent and n is a, c, t, g, u, or modified forms thereof
  misc_feature (8)..(9): where n at each of positions 8-9 can be present or absent and n is a, c, t, g, u, or modified forms thereof
  misc_feature (12)..(12): where n at position 12 can be present or absent and n is a, c, t, g, u, or modified forms thereof
  misc_feature (14)..(18): where n at each of positions 14-18 can be present or absent and n is a, c, t, g, u, or modified forms thereof
  misc_feature (21)..(22): where n at each of positions 21-22 can be present or absent and n is a, c, t, g, u, or modified forms thereof
  misc_feature (24)..(24): where n at position 24 can be present or absent and n is a, c, t, g, u, or modified forms thereof
  misc_feature (26)..(31): where n at each of positions 26-31 can be present or absent and n is a, c, t, g, u, or modified forms thereof
  misc_feature (33)..(33): where n at position 33 can be present or absent and n is a, c, t, g, u, or modified forms thereof
  misc_feature (37)..(37): where n is any useful linker
  misc_feature (38)..(48): where n at each of positions 38-48 can be present or absent and n is a, c, t, g, u, or modified forms thereof
  misc_feature (50)..(50): where n at position 50 can be present or absent and n is a, c, t, g, u, or modified forms thereof
  misc_feature (52)..(53): where n at each of positions 52-53 can be present or absent and n is a, c, t, g, u, or modified forms thereof
  misc_feature (56)..(62): where n at each of positions 56-62 can be present or absent and n is a, c, t, g, u, or modified forms thereof
  misc_feature (65)..(68): where n at each of positions 65-68 can be present or absent and n is a, c, t, g, u, or modified forms thereof
  misc_feature (70)..(71): where n at each of positions 70-71 can be present or absent and n is a, c, t, g, u, or modified forms thereof
guuuungnncununnnnnuunnanunnnnnnanaacnnnnnnnnnnnnanunnaannnnnnnagnnnnunnaaa

SEQ ID NO: 87 - DNA, 50 bp, Artificial Sequence
  Synthetic construct
  misc_feature (6)..(6): where n at position 6 can be present or absent and n is a, c, t, g, u, or modified forms thereof
  misc_feature (8)..(9): where n at each of positions 8-9 can be present or absent and n is a, c, t, g, u, or modified forms thereof
  misc_feature (12)..(12): where n at position 12 can be present or absent and n is a, c, t, g, u, or modified forms thereof
  misc_feature (13)..(13): where n is any useful linker
  misc_feature (14)..(24): where n at each of positions 14-24 can be present or absent and n is a, c, t, g, u, or modified forms thereof
  misc_feature (26)..(26): where n at position 26 can be present or absent and n is a, c, t, g, u, or modified forms thereof
  misc_feature (28)..(29): where n at each of positions 28-29 can be present or absent and n is a, c, t, g, u, or modified forms thereof
  misc_feature (32)..(38): where n at each of positions 32-38 can be present or absent and n is a, c, t, g, u, or modified forms thereof
  misc_feature (41)..(44): where n at each of positions 41-44 can be present or absent and n is a, c, t, g, u, or modified forms thereof
  misc_feature (46)..(47): where n at each of positions 46-47 can be present or absent and n is a, c, t, g, u, or modified forms thereof
guuuungnncunnnnnnnnnnnnnanunnaannnnnnnagnnnnunnaaa

SEQ ID NO: 88 - DNA, 26 bp, Artificial Sequence
  Synthetic construct
  misc_feature (6)..(6): where n at position 6 can be present or absent and n is a, c, t, g, u, or modified forms thereof
  misc_feature (8)..(9): where n at each of positions 8-9 can be present or absent and n is a, c, t, g, u, or modified forms thereof
  misc_feature (12)..(12): where n at position 12 can be present or absent and n is a, c, t, g, u, or modified forms thereof
  misc_feature (13)..(13): where n is any useful linker
  misc_feature (14)..(14): where n at position 14 can be present or absent and n is a, c, t, g, u, or modified forms thereof
  misc_feature (17)..(20): where n at each of positions 17-20 can be present or absent and n is a, c, t, g, u, or modified forms thereof
  misc_feature (22)..(23): where n at each of positions 22-23 can be present or absent and n is a, c, t, g, u, or modified forms thereof
guuuungnncunnnagnnnnunnaaa

SEQ ID NO: 89 - DNA, 52 bp, Artificial Sequence
  Synthetic construct
  misc_feature (6)..(6): where n at position 6 can be present or absent and n is a, c, t, g, u, or modified forms thereof
  misc_feature (8)..(9): where n at each of positions 8-9 can be present or absent and n is a, c, t, g, u, or modified forms thereof
  misc_feature (12)..(12): where n at position 12 can be present or absent and n is a, c, t, g, u, or modified forms thereof
  misc_feature (13)..(13): where n is any useful linker
  misc_feature (14)..(21): where n at each of positions 14-21 can be present or absent and n is a, c, t, g, u, or modified forms thereof
  misc_feature (24)..(26): where n at each of positions 24-26 can be present or absent and n is a, c, t, g, u, or modified forms thereof
  misc_feature (29)..(29): where n at position 29 can be present or absent and n is a, c, t, g, u, or modified forms thereof
  misc_feature (32)..(34): where n at each of positions 32-34 can be present or absent and n is a, c, t, g, u, or modified forms thereof
  misc_feature (36)..(36): where n at position 36 can be present or absent and n is a, c, t, g, u, or modified forms thereof
  misc_feature (45)..(47): where n at each of positions 45-47 can be present or absent and n is a, c, t, g, u, or modified forms thereof
  misc_feature (49)..(49): where n at position 49 can be present or absent and n is a, c, t, g, u, or modified forms thereof
guuuungnncunnnnnnnnnngcnnnagnuannnanauaaggcunnnunccg

SEQ ID NO: 90 - DNA, 76 bp, Artificial Sequence
  Synthetic construct
  misc_feature (1)..(2): where n at each of positions 1-2 can be present or absent and n is a, c, t, g, u, or modified forms thereof
  misc_feature (7)..(7): where n at position 7 can be present or absent and n is a, c, t, g, u, or modified forms thereof
  misc_feature (9)..(10): where n at each of positions 9-10 can be present or absent and n is a, c, t, g, u, or modified forms thereof
  misc_feature (12)..(12): where n at position 12 can be present or absent and n is a, c, t, g, u, or modified forms thereof
  misc_feature (15)..(15): where n at position 15 can be present or absent and n is a, c, t, g, u, or modified forms thereof
  misc_feature (21)..(24): where n at each of positions 21-24 can be present or absent and n is a, c, t, g, u, or modified forms thereof
  misc_feature (26)..(26): where n at position 26 can be present or absent and n is a, c, t, g, u, or modified forms thereof
  misc_feature (28)..(28): where n at position 28 can be present or absent and n is a, c, t, g, u, or modified forms thereof
  misc_feature (30)..(32): where n at each of positions 30-32 can be present or absent and n is a, c, t, g, u, or modified forms thereof
  misc_feature (39)..(39): where n is any useful linker
  misc_feature (40)..(50): where n at each of positions 40-50 can be present or absent and n is a, c, t, g, u, or modified forms thereof
  misc_feature (52)..(52): where n at position 52 can be present or absent and n is a, c, t, g, u, or modified forms thereof
  misc_feature (54)..(55): where n at each of positions 54-55 can be present or absent and n is a, c, t, g, u, or modified forms thereof
  misc_feature (58)..(64): where n at each of positions 58-64 can be present or absent and n is a, c, t, g, u, or modified forms thereof
  misc_feature (67)..(70): where n at each of positions 67-70 can be present or absent and n is a, c, t, g, u, or modified forms thereof
  misc_feature (72)..(73): where n at each of positions 72-73 can be present or absent and n is a, c, t, g, u, or modified forms thereof
nnaacanunnunuancaaaunnnnunancnnnugaaacnnnnnnnnnnnnanunnaannnnnnnagnnnnunnaaa

SEQ ID NO: 91 - DNA, 56 bp, Artificial Sequence
  Synthetic construct
  misc_feature (1)..(2): where n at each of positions 1-2 can be present or absent and n is a, c, t, g, u, or modified forms thereof
  misc_feature (7)..(7): where n at position 7 can be present or absent and n is a, c, t, g, u, or modified forms thereof
  misc_feature (9)..(10): where n at each of positions 9-10 can be present or absent and n is a, c, t, g, u, or modified forms thereof
  misc_feature (12)..(12): where n at position 12 can be present or absent and n is a, c, t, g, u, or modified forms thereof
  misc_feature (15)..(15): where n at position 15 can be present or absent and n is a, c, t, g, u, or modified forms thereof
  misc_feature (17)..(17): where n is any useful linker
  misc_feature (18)..(19): where n at each of positions 18-19 can be present or absent and n is a, c, t, g, u, or modified forms thereof
  misc_feature (21)..(28): where n at each of positions 21-28 can be present or absent and n is a, c, t, g, u, or modified forms thereof
  misc_feature (30)..(32): where n at each of positions 30-32 can be present or absent and n is a, c, t, g, u, or modified forms thereof
  misc_feature (34)..(38): where n at each of positions 34-38 can be present or absent and n is a, c, t, g, u, or modified forms thereof
  misc_feature (41)..(42): where n at each of positions 41-42 can be present or absent and n is a, c, t, g, u, or modified forms thereof
  misc_feature (45)..(50): where n at each of positions 45-50 can be present or absent and n is a, c, t, g, u, or modified forms thereof
  misc_feature (52)..(56): where n at each of positions 52-56 can be present or absent and n is a, c, t, g, u, or modified forms thereof
nnaacanunnunuancnnnunnnnnnnnunnnannnnnuunnuannnnnnunnnnn

SEQ ID NO: 92 - DNA, 33 bp, Artificial Sequence
  Synthetic construct
  misc_feature (1)..(2): where n at each of positions 1-2 can be present or absent and n is a, c, t, g, u, or modified forms thereof
  misc_feature (7)..(7): where n at position 7 can be present or absent and n is a, c, t, g, u, or modified forms thereof
  misc_feature (9)..(10): where n at each of positions 9-10 can be present or absent and n is a, c, t, g, u, or modified forms thereof
  misc_feature (12)..(12): where n at position 12 can be present or absent and n is a, c, t, g, u, or modified forms thereof
  misc_feature (15)..(15): where n at position 15 can be present or absent and n is a, c, t, g, u, or modified forms thereof
  misc_feature (17)..(17): where n is any useful linker
  misc_feature (18)..(19): where n at each of positions 18-19 can be present or absent and n is a, c, t, g, u, or modified forms thereof
  misc_feature (22)..(27): where n at each of positions 22-27 can be present or absent and n is a, c, t, g, u, or modified forms thereof
  misc_feature (29)..(33): where n at each of positions 29-33 can be present or absent and n is a, c, t, g, u, or modified forms thereof
nnaacanunnunuancnnnuannnnnnunnnnn

SEQ ID NO: 93 - DNA, 56 bp, Artificial Sequence
  Synthetic construct
  misc_feature (1)..(2): where n at each of positions 1-2 can be present or absent and n is a, c, t, g, u, or modified forms thereof
  misc_feature (7)..(7): where n at position 7 can be present or absent and n is a, c, t, g, u, or modified forms thereof
  misc_feature (9)..(10): where n at each of positions 9-10 can be present or absent and n is a, c, t, g, u, or modified forms thereof
  misc_feature (12)..(12): where n at position 12 can be present or absent and n is a, c, t, g, u, or modified forms thereof
  misc_feature (15)..(15): where n at position 15 can be present or absent and n is a, c, t, g, u, or modified forms thereof
  misc_feature (17)..(17): where n is any useful linker
  misc_feature (18)..(25): where n at each of positions 18-25 can be present or absent and n is a, c, t, g, u, or modified forms thereof
  misc_feature (28)..(30): where n at each of positions 28-30 can be present or absent and n is a, c, t, g, u, or modified forms thereof
  misc_feature (33)..(33): where n at position 33 can be present or absent and n is a, c, t, g, u, or modified forms thereof
  misc_feature (36)..(38): where n at each of positions 36-38 can be present or absent and n is a, c, t, g, u, or modified forms thereof
  misc_feature (40)..(40): where n at position 40 can be present or absent and n is a, c, t, g, u, or modified forms thereof
  misc_feature (49)..(51): where n at each of positions 49-51 can be present or absent and n is a, c, t, g, u, or modified forms thereof
  misc_feature (53)..(53): where n at position 53 can be present or absent and n is a, c, t, g, u, or modified forms thereof
nnaacanunnunuancnnnnnnnnngcnnnagnuannnanauaaggcunnnunccg

SEQ ID NO: 94-99 - skipped (6 entries)

SEQ ID NO: 100 - DNA, 218 bp, Artificial Sequence
  Synthetic construct
  misc_feature (1)..(80): where n at each of positions 1-80 can be present or absent such that this region can contain anywhere from 12 to 80 nucleotides and n is a, c, t, g, u, or modified forms thereof
  misc_feature (93)..(192): where n at each of positions 93-192 can be present or absent such that this region can contain anywhere from 3 to 100 nucleotides and n is a, c, t, g, u, or modified forms thereof
nnnnnnnnnnnnnnnnnnnnnnnnnnnnnnnnnnnnnnnnnnnnnnnnnnnnnnnnnnnnnnnnnnnnnnnnnnnnnnnnguuuuagagcuannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnuagcaaguuaaaauaaggcuaguccg

SEQ ID NO: 101 - DNA, 219 bp, Artificial Sequence
  Synthetic construct
  misc_feature (1)..(80): where n at each of positions 1-80 can be present or absent such that this region can contain anywhere from 12 to 80 nucleotides and n is a, c, t, g, u, or modified forms thereof
  misc_feature (93)..(192): where n at each of positions 93-192 can be present or absent such that this region can contain anywhere from 3 to 100 nucleotides and n is a, c, t, g, u, or modified forms thereof
nnnnnnnnnnnnnnnnnnnnnnnnnnnnnnnnnnnnnnnnnnnnnnnnnnnnnnnnnnnnnnnnnnnnnnnnnnnnnnnnguuuuagagcuannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnuagcaaguuaaaauaaggcuuuguccg

SEQ ID NO: 102 - DNA, 163 bp, Artificial Sequence
  Synthetic construct
  misc_feature (1)..(80): where n at each of positions 1-80 can be present or absent such that this region can contain anywhere from 12 to 80 nucleotides and n is a, c, t, g, u, or modified forms thereof
nnnnnnnnnnnnnnnnnnnnnnnnnnnnnnnnnnnnnnnnnnnnnnnnnnnnnnnnnnnnnnnnnnnnnnnnnnnnnnnnguuuuagagcuagaaauagcaaguuaaaauaaggcuaguccguuaucaacuugaaaaaguggcaccgagucggugcuuuuuuu

SEQ ID NO: 103 - DNA, 163 bp, Artificial Sequence; identical to SEQ ID NO: 102
  Synthetic construct
  misc_feature (1)..(80): where n at each of positions 1-80 can be present or absent such that this region can contain anywhere from 12 to 80 nucleotides and n is a, c, t, g, u, or modified forms thereof

SEQ ID NO: 104-109 - skipped (6 entries)

SEQ ID NO: 110 - protein, 1368 aa, Streptococcus pyogenes [sequence omitted]

SEQ ID NO: 111 - protein, 1368 aa, Artificial Sequence
  Synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 112 - protein, 1368 aa, Streptococcus pyogenes
MDKKYSIGLDIGTNSVGWAVITDDYKVPSKKLKGLGNTDRHGIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLADSTDKVDLRLIYLALAHMIKFRGHFLIEGDLNPDNSDVDKLFIQLVQTYNQLFEENPINASRVDAKAILSARLSKSRRLENLIAQLPGEKKNGLFGNLIALSLGLTPNFKSNFDLAEDAKLQLSKDTYDDDLDNLLAQIGDQYADLFLAAKNLSDATLLSDILRVNSEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPYQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSDILKEYPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKVGFIKRQLVETRQITKHVAQILDSRMNTKYDENDKLIREVRVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKDPIDFLEAKGYKEVRKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 113 - protein, 1629 aa, Francisella tularensis
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 114 - protein, 1409 aa, Streptococcus thermophilus
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 115 - protein, 1388 aa, Streptococcus thermophilus
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 116 - protein, 1334 aa, Listeria innocua
MKKPYTIGLDIGTNSVGWAVLTDQYDLVKRKMKIAGDSEKKQIKKNFWGVRLFDEGQTAADRRMARTARRRIERRRNRISYLQGIFAEEMSKTDANFFCRLSDSFYVDNEKRNSRHPFFATIEEEVEYHKNYPTIYHLREELVNSSEKADLRLVYLALAHIIKYRGNFLIEGALDTQNTSVDGIYKQFIQTYNQVFASGIEDGSLKKLEDNKDVAKILVEKVTRKEKLERILKLYPGEKSAGMFAQFISLIVGSKGNFQKPFDLIEKSDIECAKDSYEEDLESLLALIGDEYAELFVAAKNAYSAVVLSSIITVAETETNAKLSASMIERFDTHEEDLGELKAFIKLHLPKHYEEIFSNTEKHGYAGYIDGKTKQADFYKYMKMTLENIEGADYFIAKIEKENFLRKQRTFDNGAIPHQLHLEELEAILHQQAKYYPFLKENYDKIKSLVTFRIPYFVGPLANGQSEFAWLTRKADGEIRPWNIEEKVDFGKSAVDFIEKMTNKDTYLPKENVLPKHSLCYQKYLVYNELTKVRYINDQGKTSYFSGQEKEQIFNDLFKQKRKVKKKDLELFLRNMSHVESPTIEGLEDSFNSSYSTYHDLLKVGIKQEILDNPVNTEMLENIVKILTVFEDKRMIKEQLQQFSDVLDGVVLKKLERRHYTGWGRLSAKLLMGIRDKQSHLTILDYLMNDDGLNRNLMQLINDSNLSFKSIIEKEQVTTADKDIQSIVADLAGSPAIKKGILQSLKIVDELVSVMGYPPQTIVVEMARENQTTGKGKNNSRPRYKSLEKAIKEFGSQILKEHPTDNQELRNNRLYLYYLQNGKDMYTGQDLDIHNLSNYDIDHIVPQSFITDNSIDNLVLTSSAGNREKGDDVPPLEIVRKRKVFWEKLYQGNLMSKRKFDYLTKAERGGLTEADKARFIHRQLVETRQITKNVANILHQRFNYEKDDHGNTMKQVRIVTLKSALVSQFRKQFQLYKVRDVNDYHHAHDAYLNGVVANTLLKVYPQLEPEFVYGDYHQFDWFKANKATAKKQFYTNIMLFFAQKDRIIDENGEILWDKKYLDTVKKVMSYRQMNIVKKTEIQKGEFSKATIKPKGNSSKLIPRKTNWDPMKYGGLDSPNMAYAVVIEYAKGKNKLVFEKKIIRVTIMERKAFEKDEKAFLEEQGYRQPKVLAKLPKYTLYECEEGRRRMLASANEAQKGNQQVLPNHLVTLLHHAANCEVSDGKSLDYIESNREMFAELLAHVSEFAKRYTLAEANLNKINQLFEQNKEGDIKAIAQSFVDLMAFNAMGAPASFKFFETTIERKRYNNLKELLNSTIIYQSITGLYESRKRLDD

SEQ ID NO: 117 - protein, 1059 aa, Wolinella succinogenes
MIERILGVDLGISSLGWAIVEYDKDDEAANRIIDCGVRLFTAAETPKKKESPNKARREARGIRRVLNRRRVRMNMIKKLFLRAGLIQDVDLDGEGGMFYSKANRADVWELRHDGLYRLLKGDELARVLIHIAKHRGYKFIGDDEADEESGKVKKAGVVLRQNFEAAGCRTVGEWLWRERGANGKKRNKHGDYEISIHRDLLVEEVEAIFVAQQEMRSTIATDALKAAYREIAFFVRPMQRIEKMVGHCTYFPEERRAPKSAPTAEKFIAISKFFSTVIIDNEGWEQKIIERKTLEELLDFAVSREKVEFRHLRKFLDLSDNEIFKGLHYKGKPKTAKKREATLFDPNEPTELEFDKVEAEKKAWISLRGAAKLREALGNEFYGRFVALGKHADEATKILTYYKDEGQKRRELTKLPLEAEMVERLVKIGFSDFLKLSLKAIRDILPAMESGARYDEAVLMLGVPHKEKSAILPPLNKTDIDILNPTVIRAFAQFRKVANALVRKYGAFDRVHFELAREINTKGEIEDIKESQRKNEKERKEAADWIAETSFQVPLTRKNILKKRLYIQQDGRCAYTGDVIELERLFDEGYCEIDHILPRSRSADDSFANKVLCLARANQQKTDRTPYEWFGHDAARWNAFETRTSAPSNRVRTGKGKIDRLLKKNFDENSEMAFKDRNLNDTRYMARAIKTYCEQYWVFKNSHTKAPVQVRSGKLTSVLRYQWGLESKDRESHTHHAVDAIIIAFSTQGMVQKLSEYYRFKETHREKERPKLAVPLANFRDAVEEATRIENTETVKEGVEVKRLLISRPPRARVTGQAHEQTAKPYPRIKQVKNKKKWRLAPIDEEKFESFKADRVASANQKNFYETSTIPRVDVYHKKGKFHLVPIYLHEMVLNELPNLSLGTNPEAMDENFFKFSIFKDDLISIQTQGTPKKPAKIIMGYFKNMHGANMVLSSINNSPCEGFTCTPVSMDKKHKDKCKLCPEENRIAGRCLQGFLDYWSQEGLRPPRKEFECDQGVKFALDVKKYQIDPLGYYYEVKQEKRLGTIPQMRSAKKLVKK

SEQ ID NO: 118-200 - skipped (83 entries)

SEQ ID NO: 201 - protein, 334 aa, Nannochloropsis gaditana
MAGGGNWEFQYYTNNRSNSFVEEGVLYLQPTLTEETIGEANMMGEKPFRFDMWGMWPSDACTSNAFYGCERISDAGAQLVINPVQSARLRTTGTFTFQYGRLEVEAKLPRGDWLWPAIWLLPEKNVYGQWPASGEIDVMESRGNKPGYVKGGYDSFGSCMHWGPYFALDKYEMTCESFTLPEGKGTFNDDFHVFGMVWNEQGLYTYLDREDQKVLEVKFDRPFFERGDFADVPGTGNPWIGRPNAAPFDQPFYLVLNVAVGGLSNFFEDGDDGKPWTNTGKGAPYLFAKAKDEWYPSWAGRDSALQVKSVRVWQKPGQGKASARNPEKARVWVA

SEQ ID NO: 202 - protein, 871 aa, Nannochloropsis gaditana
MVPARVCGACKGQLEDEDLKDRIVWRMVRVEAFLKDRLIPYFAPGDHTQLDRALRTVGGWVSRAARRAPPLRSTTALAGEALELFSRYGYAGVAGVLLRHEHVEAVELLKEVSGVDAAWPVTGSQLSAAMYYLLARGRGERGAAPDAEQEAHRGCPPASDSLMQDLLDVAPLALHFAYCDNLVEMQLKAQQQGWRLVFAYAPPAAQAGQPAFVLLCHLTEKEACLVVRGPDRAQDVLVDIRGLPMPFPLAGEGAGSGEKGSQDKESGWANVSTEWMASCGAAEAGHWLFSEVYPHLHRLAKEGYSLTLAGHSVGGAVAALLGVLMREEGMTEGLRCYTFGSPACVNQKLAQVCEAFVTTVVLHDDVIPRVTPTGVRGLLKDLLSERERAEQHWQDDVEAIIVRSKGHWAPRCDDDLGNYWGVGCTRDVVSVGSGAASNRYSIRHEDGTTTTVNLALARRRLLDSGGDAAADRGSSVSKNVTGACPAPCGLGEAGVNSALTSSGTSVPSLGAASSPGAESLGDGDDTDDWGEDGGEGAERAGEEAQAWMGDRTGSLQEGESEGEEGEELQGRWLGSRDAPPASSDGMGAEEEGGAGLEQSLALWNLFGSEGSEAAAAAPGREPDSRAVLEVEGVPVDTKQVSVSTTAPTAAADSTCSFSSSTSSLSSSSPSPPAPEGGREGGSKSEEKEEGNEEEEEEEEEEEEEEEEEDCGILVEDVSLPELYPPGRLVHIYSYRGVYKACMPPRSFPGLRRIPLQGNLLKDHAPTAYFSALCEVIDVRRAPQPPPAWEGFKEKEACVCCAVDLTWQRATASEAHRDREKHNCRCCGGLVCQDCSRHRRALPSIGLSAPARVCDRCFFGGKQSAVEATERG

SEQ ID NO: 203 - protein, 473 aa, Nannochloropsis gaditana
MLSWRADDSDLPQSVTIKSTIAKLEESLQAKDTSKLQFILLNGLLKRNHLGIDERDLHIRALSGSKLLVERYDKKVVECIKYITECQELTREEKVMFVKKARRALGQTALMLSGGGSISMYHAGPGPFTNTVADLQMVCRGDAPNVTLLALHESKHTPSSKVRVKNIRISSFPSSPGTGVVRALITEGLYRHIRVISGASGGSIIAGMAAIHNEKELMDRVLVKEVSTDFKHNGEMRQKKIVWFPPLFEQAKHFIKNGILIDNKEFQRTCEFYYGSFTFQEAFERTHKHVCISVAASTLGASSQGGPRRLLLNHITTPNVLIRSAVAASCALPGIMAPNYLQCKDDRGHVVPFDMDGVQYVDGSLQADLPFRRISTLFAVSHFIVSQVNFHVVPFLRKMHSPAESSLYWKLFRFFDTDIRHRVTSLAELGLLPRVFGQDLSGIFRQRYSGHVTLTPRFRMSEMIGLKAFQVKS

SEQ ID NO: 204 - protein, 497 aa, Nannochloropsis gaditana
IFDKFFAWSSRWLFSTNHKDIGTLYLIFGGVAGIAGTTLSVLIRLELAQPGNQFLSGNNQLYNVIVTGHAFIMIFFFVMPVLIGGFGNWFVPLMIGAPDMAFPRMNNISFWLLPPSLILLLASTFVEAGAGTGWTVYPPLSGAQAHSGPSVDLAIFSLHLSGAASILGAINFITTIFNMRAPGMNMHRLPLFVWSVLITAFLLLLSLPVFAGAITMLLTDRNFNTTFYDPAGGGDPVLYQHLFWFFGHPEVYILILPAFGIISHIVSSFANKPVFGYLGMIYAMLSIGVLGFIVWAHHMYTVGLDIDTRAYFTAATMIIAVPTGIKIFSWVATMWGGFIELKTPMLFAIGFIFLFTVGGVTGVVLANSGIDVALHDTYYVIAHFHYVLSMGAVFGIFAGFYFWIKKITGLDYPEVLGQIHFWIFFFGVNITFFPMHFLGLAGMPRRIPDYPDAYSGWNAIASFGSYLSALSAIFFFYVVYITLTEKGKEDTLKFRTI

SEQ ID NO: 205 - protein, 497 aa, Nannochloropsis gaditana
IFDKFFAWSSRWLFSTNHKDIGTLYLIFGAIAGVAGTTLSVLIRLELAQPGNQFLSGNNQLYNVIVTGHAFIMIFFFVMPVLIGGFGNWFVPLMIGAPDMAFPRMNNISFWLLPPSLILLLASTFVEAGAGTGWTVYPPLSGAQAHSGPSVDLAIFSLHLSGAASILGAINFITTIFNMRAPGMNMHRLPLFVWSVLITAFLLLLSLPVFAGAITMLLTDRNFNTTFYDPAGGGDPVLYQHLFWFFGHPEVYILILPAFGIISHIVSSFANKPVFGYLGMIYAMLSIGVLGFIVWAHHMYTVGLDIDTRAYFTAATMIIAVPTGIKIFSWVATMWGGFIELKTPMLFAIGFIFLFTVGGVTGVVLANSGIDVALHDTYYVIAHFHYVLSMGAVFGIFAGFYFWIKKITGLDYPEVLGQIHFWIFFFGVNITFFPMHFLGLAGMPRRIPDYPDAYSGWNAIASFGSYLSALSAIFFFYVVYITLTEKGKEDTFKFRTI

SEQ ID NO: 206 - protein, 320 aa, Nannochloropsis gaditana
QHSNRISSLTQPHYLSTMLARAVLPTRSGSLAAAFLKTSSATIMPPKQLQGLSRTLQVKSYRQSTVFYRAMSTTLKPEERAGTFTPAAPSTTTQEKEELRDGARSIIHFKLSPNRHALNVPKLDPKEKVWENPTHHSVWTKEEVENVEVTHLPPADWTSRVAYTIAQTLRFSFDVLAGFKFRKATEDMYLNRMVFLETVAVFFLSYLINPKICHRLVGHIEEEAVRTYTHIIEEMDAGQLPLFNHVIPPPIAVSYWKLAPDATFRDLLLAIRKDEATHREVNHTFANLKENDDNPFLAEEEYRAKITTMGQPTPVAEKKA

SEQ ID NO: 207 - protein, 499 aa, Escherichia coli
MSILYEERLDGALPDVDRTSVLMALREHVPGLEILHTDEEIIPYECDGLSAYRTRPLLVVLPKQMEQVTAILAVCHRLRVPVVTRGAGTGLSGGALPLEKGVLLVMARFKEILDINPVGRRARVQPGVRNLAISQAVAPHNLYYAPDPSSQIACSIGGNVAENAGGVHCLKYGLTVHNLLKIEVQTLDGEALTLGSDALDSPGFDLLALFTGSEGMLGVTTEVTVKLLPKPPVARVLLASFDSVEKAGLAVGDIIANGIIPGGLEMMDNLSIRAAEDFIHAGYPVDAEAILLCELDGVESDVQEDCERVNDILLKAGATDVRLAQDEAERVRFWAGRKNAFPAVGRISPDYYCMDGTIPRRALPGVLEGIARLSQQYDLRVANVFHAGDGNMHPLILFDANEPGEFARAEELGGKILELCVEVGGSISGEHGIGREKINQMCAQFNSDEITTFHAVKAAFDPDGLLNPGKNIPTLHRCAEFGAMHVHHGHLPFPELERF

SEQ ID NO: 208 - protein, 350 aa, Escherichia coli
MLRECDYSQALLEQVNQAISDKTPLVIQGSNSKAFLGRPVTGQTLDVRCHRGIVNYDPTELVITARVGTPLVTIEAALESAGQMLPCEPPHYGEEATWGGMVACGLAGPRRPWSGSVRDFVLGTRIITGAGKHLRFGGEVMKNVAGYDLSRLMVGSYGCLGVLTEISMKVLPRPRASLSLRREISLQEAMSEIAEWQLQPLPISGLCYFDNALWIRLEGGEGSVKAARELLGGEEVAGQFWQQLREQQLPFFSLPGTLWRISLPSDAPMMDLPGEQLIDWGGALRWLKSTAEDNQIHRIARNAGGHATRFSAGDGGFAPLSAPLFRYHQQLKQQLDPCGVFNPGRMYAEL

SEQ ID NO: 209 - protein, 407 aa, Escherichia coli
MQTQLTEEMRQNARALEADSILRACVHCGFCTATCPTYQLLGDELDGPRGRIYLIKQVLEGNEVTLKTQEHLDRCLTCRNCETTCPSGVRYHNLLDIGRDIVEQKVKRPLPERILREGLRQVVPRPAVFRALTQVGLVLRPFLPEQVRAKLPAETVKAKPRPPLRHKRRVLMLEGCAQPTLSPNTNAATARVLDRLGISVMPANEAGCCGAVDYHLNAQEKGLARARNNIDAWWPAIEAGAEAILQTASGCGAFVKEYGQMLKNDALYADKARQVSELAVDLVELLREEPLEKLAIRGDKKLAFHCPCTLQHAQKLNGEVEKVLLRLGFTLTDVPDSHLCCGSAGTYALTHPDLARQLRDNKMNALESGKPEMIVTANIGCQTHLASAGRTSVRHWIEIVEQALEKE

SEQ ID NO: 210 - protein, 12 aa, Artificial Sequence
  Synthetic construct
RRRRRRRRRRRR

SEQ ID NO: 211 - protein, 9 aa, Artificial Sequence
  Synthetic construct
RRRRRRRRR

SEQ ID NO: 212 - protein, 18 aa, Artificial Sequence
  Synthetic construct
KHKHKHKHKHKHKHKHKH

SEQ ID NO: 213 - protein, 9 aa, Artificial Sequence
  Synthetic construct
RKKRRQRRR

SEQ ID NO: 214 - protein, 12 aa, Artificial Sequence
  Synthetic construct
GRKKRRQRRRPQ

SEQ ID NO: 215 - protein, 10 aa, Artificial Sequence
  Synthetic construct
GRKKRRQRRR

SEQ ID NO: 216 - protein, 13 aa, Artificial Sequence
  Synthetic construct
GRKKRRQRRRPPQ

SEQ ID NO: 217 - protein, 11 aa, Artificial Sequence
  Synthetic construct
YGRKKRRQRRR

SEQ ID NO: 218 - protein, 18 aa, Artificial Sequence
  Synthetic construct
RKKRRQRRRRKKRRQRRR

SEQ ID NO: 219 - protein, 21 aa, Artificial Sequence
  Synthetic construct
  MOD_RES (1)..(1): Optional acetylation
  MOD_RES (21)..(21): Cysteamide
GLWRALWRLLRSLWRLLWRAX

SEQ ID NO: 220 - protein, 18 aa, Artificial Sequence
  Synthetic construct
RQIKIWFQNRRMKWKKGG

SEQ ID NO: 221 - protein, 16 aa, Artificial Sequence
  Synthetic construct
RQIRIWFQNRRMRWRR

SEQ ID NO: 222 - protein, 16 aa, Artificial Sequence
  Synthetic construct
RQIKIWFQNRRMKWKK

SEQ ID NO: 223 - protein, 17 aa, Artificial Sequence
  Synthetic construct
CSIPPEVKFNKPFVYLI

SEQ ID NO: 224 - protein, 13 aa, Artificial Sequence
  Synthetic construct
  MOD_RES (13)..(13): Optional amidation
FVQWFSKFLGRIL

SEQ ID NO: 225 - protein, 18 aa, Artificial Sequence
  Synthetic construct
KLALKLALKALKAALKLA

SEQ ID NO: 226 - protein, 9 aa, Artificial Sequence
  Synthetic construct
RRWWRRWRR

SEQ ID NO: 227 - protein, 18 aa, Artificial Sequence
  Synthetic construct
LLIILRRRIRKQAHAHSK

SEQ ID NO: 228 - protein, 27 aa, Artificial Sequence
  Synthetic construct
GWTLNSAGYLLGKINLKALAALAKKIL

SEQ ID NO: 229 - protein, 27 aa, Artificial Sequence
  Synthetic construct
GALFLGFLGAAGSTMGAWSQPKKKRKV

SEQ ID NO: 230 - protein, 21 aa, Artificial Sequence
  Synthetic construct
KETWWETWWTEWSQPKKKRKV

SEQ ID NO: 231 - protein, 22 aa, Artificial Sequence
  Synthetic construct
  MOD_RES (1)..(1): Optional acetylation
  MOD_RES (22)..(22): Cysteamine
KETWWETWWTEWSQPKKKRKVX

SEQ ID NO: 232 - protein, 11 aa, Artificial Sequence
  Synthetic construct
  MOD_RES (11)..(11): Optional amidation
WKLFKKILKVL

SEQ ID NO: 233 - protein, 11 aa, Artificial Sequence
  Synthetic construct
KKLFKKILKYL

SEQ ID NO: 234 - protein, 11 aa, Artificial Sequence
  Synthetic construct
  MOD_RES (11)..(11): Optional amidation
KKLFKKILKYL

SEQ ID NO: 235 - protein, 10 aa, Artificial Sequence
  Synthetic construct
  MOD_RES (3)..(3): Optional acetamidomethylation
GDCLPHLKLC

SEQ ID NO: 236 - protein, 24 aa, Artificial Sequence
  Synthetic construct
LGTYTQDFNKFHTFPQTAIGVGAP

SEQ ID NO: 237 - protein, 30 aa, Artificial Sequence
  Synthetic construct
GAAEAAARVYDLGLRRLRQRRRLRRERVRA

SEQ ID NO: 238 - protein, 27 aa, Artificial Sequence
  Synthetic construct
MGLGLHLLVLAAALQGAWSQPKKKRKV

SEQ ID NO: 239 - skipped

SEQ ID NO: 240 - protein, 17 aa, Artificial Sequence
  Synthetic construct
MGGCVSTPKSCVGAKLR

SEQ ID NO: 241 - protein, 31 aa, Artificial Sequence
  Synthetic construct
MQTLTASSSVSSIQRHRPHPAGRRSSSVTFS

SEQ ID NO: 242 - protein, 15 aa, Artificial Sequence
  Synthetic construct
MKNPPSSFASGFGIR

SEQ ID NO: 243 - protein, 32 aa, Artificial Sequence
  Synthetic construct
MAALIPAIASLPRAQVEKPHPMPVSTRPGLVS

SEQ ID NO: 244 - protein, 33 aa, Artificial Sequence
  Synthetic construct
MSSPPPLFTSCLPASSPSIRRDSTSGSVTSPLR

SEQ ID NO: 245 - protein, 34 aa, Artificial Sequence
  Synthetic construct
MFSYLPRYPLRAASARALVRATRPSYRYALLRYQ

SEQ ID NO: 246 - protein, 13 aa, Artificial Sequence
  Synthetic construct
  MISC_FEATURE (1)..(1): where X1 is R, S, G, or A
  MISC_FEATURE (2)..(2): where X2 is R or A
  MISC_FEATURE (3)..(3): where X3 is R, S, V, or A
  MISC_FEATURE (4)..(4): where X4 is A, S, R, or F
  MISC_FEATURE (5)..(5): where X5 is V or L
  MISC_FEATURE (6)..(6): where X6 is V or R
  MISC_FEATURE (8)..(8): where X8 is V or R
  MISC_FEATURE (10)..(10): where X10 is A, S, R, or F
  MISC_FEATURE (11)..(11): where X11 is R or A
  MISC_FEATURE (12)..(12): where X12 is any amino acid, e.g., E, L, V, Q, A, R, and S
XXXXXXVXAXXXP

SEQ ID NO: 247 - protein, 13 aa, Artificial Sequence
  Synthetic construct
  MISC_FEATURE (1)..(1): where X1 is R or S
  MISC_FEATURE (3)..(3): where X3 is R or S
  MISC_FEATURE (4)..(4): where X4 is A or S
  MISC_FEATURE (5)..(5): where X5 is V or L
  MISC_FEATURE (10)..(10): where X10 is A or S
  MISC_FEATURE (12)..(12): X12 is any amino acid, e.g., E, L, Q, A, R, and S
XRXXXVVRAXAXP

SEQ ID NO: 248 - protein, 11 aa, Artificial Sequence
  Synthetic construct
  MISC_FEATURE (1)..(1): where X1 is G, A, or F
  MISC_FEATURE (2)..(2): where X2 is V, L, Q, or S
  MISC_FEATURE (4)..(4): where X4 is A, G, or T
  MISC_FEATURE (5)..(5): where X5 is F, S, or Y
  MISC_FEATURE (7)..(7): where X7 is T or A
  MISC_FEATURE (10)..(10): where X10 is A or S
  MISC_FEATURE (11)..(11): where X11 is any amino acid, e.g., D, A, G, S, or F
XXRXXAXAAXX

SEQ ID NO: 249 - protein, 11 aa, Artificial Sequence
  Synthetic construct
  MISC_FEATURE (1)..(1): where X1 is G, A, or F
  MISC_FEATURE (7)..(7): where X7 is T or A
  MISC_FEATURE (11)..(11): where X11 is any amino acid, e.g., D, A, G, S, or F
XVRAFAXAAAX

(19) United States
(12) Patent Application Publication
(10) Pub. No.: US 2016/0090603 A1
(43) Pub. Date: Mar. 31, 2016

(54) DELIVERY PLATFORMS FOR THE DOMESTICATION OF ALGAE AND PLANTS

(71) Applicant: Sandia Corporation, Albuquerque, NM (US)

(72) Inventors: Eric Christopher Carnes, Albuquerque, NM (US); Anne Ruffing, Albuquerque, NM (US); Carlee Erin Ashley, Albuquerque, NM (US)

(21) Appl. No.: 14/871,748

(22) Filed: Sep. 30, 2015

Related U.S. Application Data
(60) Provisional application No. 62/057,968, filed on Sep. 30, 2014.
(60) Provisional application No. 62/129,028, filed on Mar. 5, 2015.

(51) Int. Cl. C12N 15/82

(52) U.S. Cl. CPC C12N 15/8206

(57) ABSTRACT

The present invention relates to a delivery platform that can be used to genetically modify a target in a plant or an alga. In one instance, polypeptides and/or polynucleotides can be delivered using silica delivery platforms, e.g., silica carriers or protocells. Such platforms can be employed to control gene activation and repression in the plant or alga.

### CROSS-REFERENCE TO RELATED APPLICATION

This application claims the benefit of U.S. Provisional Application No. 62/057,968, filed Sep. 30, 2014, and U.S. Provisional Application No. 62/129,028, filed Mar. 5, 2015, each of which is hereby incorporated by reference in its entirety.

### STATEMENT OF GOVERNMENT INTEREST

This invention was made with Government support under contract no. DE-AC04-94AL85000 awarded by the U.S. Department of Energy to Sandia Corporation. The Government has certain rights in the invention.

### FIELD OF THE INVENTION

The present invention relates to a delivery platform that can be used to genetically modify a target in a plant or an alga. In one instance, polypeptides and/or polynucleotides can be delivered using silica delivery platforms, e.g., silica carriers or protocells. Such platforms can be employed to control gene activation and repression in the plant or alga.

### BACKGROUND OF THE INVENTION

Plants and algae can provide valuable resources for renewable energy. For instance, algal biofuels are promising candidates for renewable energy, but current algal productivities cannot provide economically-feasible fuel production. So too, plant-based biofuels would benefit from any modifications that can enhance productivity and efficiency. However, plant and algal cell walls can be recalcitrant, thereby thwarting the delivery of genetic elements that can impart improved properties. Accordingly, there is a need for improved delivery platforms that can facilitate delivery of biological or chemical agents to plants and algae.

### SUMMARY OF THE INVENTION

The present invention relates to a delivery platform that can be used to genetically modify a target (e.g., any herein) in a plant or an alga. In one instance, the delivery platform includes a CRISPR/Cas system (e.g., a type I, II, or III CRISPR/Cas system, as well as modified versions thereof, such as a CRISPR/dCas9 system).

The delivery platform can be a protocell or a carrier (e.g., a silica carrier). In one embodiment, the protocell includes a nanoparticle core, a supported lipid layer, and a cargo (e.g., a CRISPR/Cas system) encapsulated within the core (e.g., within one or more pores defined within the core). In another embodiment, the carrier (e.g., a silica carrier) includes a biological package, an inorganic shell (e.g., a silica shell) encapsulating the package, an optional supported lipid layer, and an optional cargo (e.g., within one or more pores defined within the shell, if the shell is porous; and/or in proximity to an inner surface of the shell, e.g., complexed with the biological package with a covalent or non-covalent bond). Each element of the protocell or the carrier can be modified to include one or more components that facilitate specific targeting and effective delivery of the cargo or the package.

The delivery platform can be delivered to any useful target, including a host (e.g., a plant or an alga). The delivery platform can be used to delivery one or more cargos, e.g., a CRISPR/Cas system and one or more other agents, such as a drug, an agrochemical, a nutrient, etc. Additional details follow.

### DEFINITIONS

As used herein, the term “about” means+/−10% of any recited value. As used herein, this term modifies any recited value, range of values, or endpoints of one or more ranges.

By “micro” is meant having at least one dimension that is less than 1 mm. For instance, a microstructure (e.g., any structure described herein, such as a microparticle) can have a length, width, height, cross-sectional dimension, circumference, radius (e.g., external or internal radius), or diameter that is less than 1 mm.

By “nano” is meant having at least one dimension that is less than 1 μm. For instance, a nanostructure (e.g., any structure described herein, such as a nanoparticle) can have a length, width, height, cross-sectional dimension, circumference, radius (e.g., external or internal radius), or diameter that is less than 1 μm.

The term “cargo” is used herein to describe any molecule or compound, whether a small molecule or macromolecule having an activity relevant to its use in MSNPSs, protocells, and/or carriers, especially including biological activity, which can be included in MSNPs, protocells, and/or carriers according to the present invention. In principal embodiments of the present invention, the cargo is a nucleic acid sequence, such as ds plasmid DNA. The cargo may be included within the pores and/or on the surface of the MSNP according to the present invention. Additional representative cargo may include, for example, a small molecule bioactive agent, a nucleic acid (e.g., RNA or DNA), a polypeptide, including a protein or a carbohydrate. Particular examples of such cargo include RNA, such as mRNA, siRNA, shRNA micro RNA, a polypeptide or protein, including a protein toxin (e.g., ricin toxin A-chain or diphtheria toxin A-chain), and/or DNA (including double stranded or linear DNA, complementary DNA (cDNA), minicircle DNA, naked DNA and plasmid DNA, which optionally may be supercoiled and/or packaged (e.g., with histones) and which may be optionally modified with a nuclear localization sequence). Cargo may also include a reporter as described herein.

The term “effective” is used herein, unless otherwise indicated, to describe an amount of a compound, composition or component which, when used within the context of its use, produces or effects an intended result. The term effective subsumes all other effective amount or effective concentration terms (including the term “therapeutically effective”) which are otherwise described or used in the present application.

By “salt” is meant an ionic form of a compound or structure (e.g., any formulas, compounds, or compositions described herein), which includes a cation or anion compound to form an electrically neutral compound or structure. Salts are well known in the art. For example, non-toxic salts, pharmaceutically acceptable salts are described in Berge S M et al., “Pharmaceutical salts,” J. Pharm. Sci. 1977 January; 66(1): 1-19; and in “Handbook of Pharmaceutical Salts: Properties, Selection, and Use,” Wiley-VCH, April 2011 (2nd rev. ed., eds. P. H. Stahl and C. G. Wermuth). The salts can be prepared in situ during the final isolation and purification of the compounds of the invention or separately by reacting the free base group with a suitable organic acid (thereby producing an anionic salt) or by reacting the acid group with a suitable metal or organic salt (thereby producing a cationic salt). Representative anionic salts include acetate, adipate, alginate, ascorbate, aspartate, benzenesulfonate, benzoate, bicarbonate, bisulfate, bitartrate, borate, bromide, butyrate, camphorate, camphorsulfonate, chloride, citrate, cyclopentanepropionate, digluconate, dihydrochloride, diphosphate, dodecylsulfate, edetate, ethanesulfonate, fumarate, glucoheptonate, glucomate, glutamate, glycerophosphate, hemisulfate, heptonate, hexanoate, hydrobromide, hydrochloride, hydroiodide, hydroxyethanesulfonate, hydroxynaphthoate, iodide, lactate, lactobionate, laurate, lauryl sulfate, malate, maleate, malonate, mandelate, mesylate, methanesulfonate, methylbromide, methylnitrate, methylsulfate, mucate, 2-naphthalenesulfonate, nicotinate, nitrate, oleate, oxalate, palmitate, pamoate, pectinate, persulfate, 3-phenylpropionate, phosphate, picrate, pivalate, polygalacturonate, propionate, salicylate, stearate, subacetate, succinate, sulfate, tannate, tartrate, theophyllinate, thiocyanate, triethiodide, toluenesulfonate, undecanoate, valerate salts, and the like. Representative cationic salts include metal salts, such as alkali or alkaline earth salts, e.g., barium, calcium (e.g., calcium edetate), lithium, magnesium, potassium, sodium, and the like; other metal salts, such as aluminum, bismuth, iron, and zinc; as well as nontoxic ammonium, quaternary ammonium, and amine cations, including, but not limited to ammonium, tetramethylammonium, tetraethylammonium, methylamine, dimethylamine, trimethylamine, triethylamine, ethylamine, pyridinium, and the like. Other cationic salts include organic salts, such as chloroprocaine, choline, dibenzylethylenediamine, dicthanolamine, ethylenediamine, methylglucamine, and procaine.

The term “mesoporous silica nanoparticles” (MSNPs) is used to describe nanoparticles according to the present invention which are modified to target specific host cells or structures therein (e.g., organelles therein). Particularly relevant MSNPs for use in the present invention are described in international patent application PCT/US2014/56312, filed Sep. 18, 2014, entitled “Core and Surface Modification of Mesoporous Silica Nanoparticles to Achieve Cell Specific Targeting in Vivo,” and application PCT/US2014/56342, also filed Sep. 18, 2014, entitled “Torroidal Mesoporous Silica Nanoparticles (TMSNPs) and Related Protocells,” both of which applications are incorporated herein in their entirety.

The phrase “effective average particle size” as used herein to describe a multiparticulate (e.g., a porous nanoparticulate) means that at least 50% of the particles therein are of a specified size. Accordingly, “effective average particle size of less than about 2,000 nm in diameter” means that at least 50% of the particles therein are less than about 2,000 nm in diameter. In certain embodiments, nanoparticulates have an effective average particle size of less than about 2,000 nm (i.e., 2 microns), less than about 1,900 nm, less than about 1.800 nm, less than about 1,700 nm, less than about 1,600 nm, less than about 1,500 nm, less than about 1,400 nm, less than about 1,300 nm, less than about 1,200 nm, less than about 1,100 nm, less than about 1,000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. In certain aspects of the present invention, the MSNPs, protocells, and/or carriers are monodisperse and generally no greater than about 50 nm in average diameter, often less than about 30 nm in average diameter, as otherwise described herein. The term “D₅₀” refers to the particle size below which 50% of the particles in a multiparticulate fall. Similarly, the term “D₉₀” refers to the particle size below which 90% of the particles in a multiparticulate fall.

The term “monodisperse” is used as a standard definition established by the National Institute of Standards and Technology (NIST) (Particle Size Characterization, Special Publication 960-1, January 2001) to describe a distribution of particle size within a population of particles, in this case nanoparticles, which particle distribution may be considered monodisperse if at least 90% of the distribution lies within 5% of the median size. See, e.g., Takeuchi S et al., Adv. Mater. 2005; 17(8): 1067-72.

The term “lipid” is used to describe the components which are used to form lipid bi- or multilayers on the surface of the particles, that are used in the present invention (e.g., as protocells or as carriers) and may include a PEGylated lipid. Various embodiments provide nanostructures, that are constructed from nanoparticles, which support one or more lipid layers (e.g., bilayer(s) or multilayer(s)). In embodiments according to the present invention, the nanostructures preferably include, for example, a core-shell structure including a porous particle core surrounded by a shell of lipid bilayer(s). The nanostructure, preferably a porous alum nanostructure as described above, supports the lipid bilayer membrane structure.

The terms “targeting ligand” and “targeting active species” are used to describe a compound or moiety (e.g., an antigen), which is complexed or covalently bonded to the surface of MSNPs, protocells, and/or carriers according to the present invention (e.g., either directly on an outer surface of a delivery platform or on a supported lipid layer disposed on an outer surface of a particle of the present invention). The targeting ligand, in turn, binds to a moiety on the surface of a cell to be targeted so that the MSNPs, protocells, and/or carriers may bind to the surface of the targeted cell, enter the cell or an organelle thereof, and/or deposit their contents into the cell. The targeting active species for use in the present invention is preferably a targeting peptide (e.g., a cell penetration peptide, a fusogenic peptide, or an endosomolytic peptide, as otherwise described herein), a polypeptide including an antibody or antibody fragment, an aptamer, or a carbohydrate, among other species that bind to a targeted cell.

The term “reporter” is used to describe an imaging agent or moiety which is incorporated into the phospholipid bilayer or cargo of MSNPs according to an embodiment of the present invention and provides a signal that can be measured. The moiety may provide a fluorescent signal or may be a radioisotope which allows radiation detection, among others. Exemplary fluorescent labels for use in MSNPs, protocells, and/or carriers (preferably via conjugation or adsorption to the lipid bi- or multilayer or the silica core or the silica shell, although these labels may also be incorporated into cargo elements such as DNA, RNA, polypeptides and small molecules which are delivered to cells by the protocells or carriers) include Hoechst 33342 (350/461), 4′,6-diamidino-2-phenylindole (DAPI, 356/451), Alexa Fluor® 405 carboxylic acid, succinimidyl ester (401/421), CellTracker™ Violet BMQC (415/516), CellTracker™ Green CMFDA (492/517), calcein (495/515), Alexa Fluor® 488 conjugate of annexin V (495/519), Alexa Fluor® 488 goat anti-mouse IgG (H+L) (495/519), Click-iT® AHA Alexa Fluor® 488 Protein Synthesis HCS Assay (495/519), LIVE/DEAD® Fixable Green Dead Cell Stain Kit (495/519), SYTOX® Green nucleic acid stain (504/523), MitoSOX™ Red mitochondrial superoxide indicator (510/580), Alexa Fluor® 532 carboxylic acid, succinimidyl ester (532/554), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608), Alexa Fluor® 647 hydrazide (649/666), Alexa Fluor® 647 carboxylic acid, succinimidyl ester (650/668), Ulysis™ Alexa Fluor® 647 Nucleic Acid Labeling Kit (650/670) and Alexa Fluor® 647 conjugate of annexin V (650/665). Moieties that enhance the fluorescent signal or slow the fluorescent fading may also be incorporated and include SlowFade® Gold antifade reagent (with and without DAPI) and Image-iT® FX signal enhancer. All of these are well known in the art.

The terms “polynucleotide” and “nucleic acid,” used interchangeably herein, refer to a polymeric form of nucleotides of any length, either ribonucleotides or deoxyribonucleotides.

Thus, this term includes, but is not limited to, single-stranded (e.g., sense or antisense), double-stranded, or multi-stranded ribonucleic acids (RNAs), deoxyribonucleic acids (DNAs), threose nucleic acids (TNAs), glycol nucleic acids (GNAs), peptide nucleic acids (PNAs), locked nucleic acids (LNAs), or hybrids thereof, genomic DNA, cDNA, DNA-RNA hybrids, or a polymer comprising purine and pyrimidine bases or other natural, chemically or biochemically modified, non-natural, or derivatized nucleotide bases. Polynucleotides can have any useful two-dimensional or three-dimensional structure or motif, such as regions including one or more duplex, triplex, quadruplex, hairpin, and/or pseudoknot structures or motifs.

The term “modified,” as used in reference to nucleic acids, means a nucleic acid sequence including one or more modifications to the nucleobase, nucleoside, nucleotide, phosphate group, sugar group, and/or internucleoside linkage (e.g., phosphodiester backbone, linking phosphate, or a phosphodiester linkage).

The nucleoside modification may include, but is not limited to, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl)adenosine, N6-glycinyl carbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and combinations thereof.

A sugar modification may include, but is not limited to, a locked nucleic acid (LNA, in which the 2′-hydroxyl is connected by a C₁₋₆ alkylene or C₁₋₆ heteroalkylene bridge to the 4′-carbon of the same ribose sugar), replacement of the oxygen in ribose (e.g., with S, Se, or alkylene, such as methylene or ethylene), addition of a double bond (e.g., to replace ribose with cyclopentenyl or cyclohexenyl), ring contraction of ribose (e.g., to form a 4-membered ring of cyclobutane or oxetane), ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone), multicyclic forms (e.g., tricyclic), and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with a-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar.

A backbone modification may include, but is not limited to, 2′-deoxy- or 2′-O-methyl modifications. A phosphate group modification may include, but is not limited to, phosphorothioate, phosphoroselenates, boranophosphates, boranophosphate esters, hydrogen phosphonates, phosphoramidates, phosphorodiamidates, alkyl or aryl phosphonates, phosphotriesters, phosphorodithioates, bridged phosphoramidates, bridged phosphorothioates, or bridged methylene-phosphonates.

“Complementarity” or “complementary” refers to the ability of a nucleic acid to form hydrogen bond(s) with another nucleic acid sequence by either traditional Watson-Crick or other non-traditional types, e.g., form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C). In addition, it is also known in the art that for hybridization between two RNA molecules (e.g., dsRNA), guanine (G) base pairs with uracil (U). A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” or “sufficient complementarity” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part 1, Second Chapter “Overview of principles of hybridization and the strategy of nucleic acid probe assay”, Elsevier, N.Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence. Hybridization and washing conditions are well known and exemplified in Sambrook J, Fritsch E F, and Maniatis T, “Molecular Cloning: A Laboratory Manual,” Second Edition, Cold Spring Harbor Laboratory Press, Cold Spring Harbor (1989), particularly Chapter 11 and Table 11.1 therein; and Sambrook J and Russell W, “Molecular Cloning: A Laboratory Manual,” Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g., complementarity over 35 or less, 30 or less, 25 or less, 22 or less, 20 or less, or 18 or less nucleotides) the position of mismatches becomes important (see Sambrook et al., supra, 11.7-11.8). Typically, the length for a hybridizable nucleic acid is at least about 10 nucleotides. Illustrative minimum lengths for a hybridizable nucleic acid are: at least about 15 nucleotides; at least about 20 nucleotides; at least about 22 nucleotides; at least about 25 nucleotides; and at least about 30 nucleotides). Furthermore, the skilled artisan will recognize that the temperature and wash solution salt concentration may be adjusted as necessary, according to factors such as length of the region of complementation and the degree of complementation.

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid sequence to which they are targeted. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST programs (basic local alignment search tools) and PowerBLAST programs known in the art (Altschul S F et al., J. Mol. Biol. 1990; 215:403-10; Zhang J et al., Genome Res. 1997; 7:649-56) or by using the Gap program (Wisconsin Sequence Analysis Package, Version 8 for Unix, Genetics Computer Group, University Research Park, Madison Wis.), using default settings, which uses the algorithm of Smith T F et al., Adv. Appl. Math. 1981; 2(4):482-9).

By “protein,” “peptide,” or “polypeptide,” as used interchangeably, is meant any chain of more than two amino acids, regardless of post-translational modification (e.g., glycosylation or phosphorylation), constituting all or part of a naturally occurring polypeptide or peptide, or constituting a non-naturally occurring polypeptide or peptide, which can include coded amino acids, non-coded amino acids, modified amino acids (e.g., chemically and/or biologically modified amino acids), and/or modified backbones.

The term “fragment” is meant a portion of a nucleic acid or a polypeptide that is at least one nucleotide or one amino acid shorter than the reference sequence. This portion contains, preferably, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, 1000, 1250, 1500, 1750, 1800 or more nucleotides; or 10, 20, 30, 40, 50, 60, 70, 80, 90, 100, 150, 200, 250, 300, 350, 400, 450, 500, 550, 600, 640 amino acids or more. In another example, any polypeptide fragment can include a stretch of at least about 5 (e.g., about 10, about 20, about 30, about 40, about 50, or about 100) amino acids that are at least about 40% (e.g., about 50%, about 60%, about 70%, about 80%, about 90%, about 95%, about 87%, about 98%, about 99%, or about 100%) identical to any of the sequences described herein can be utilized in accordance with the invention. In certain embodiments, a polypeptide to be utilized in accordance with the invention includes 2, 3, 4, 5, 6, 7, 8, 9, 10, or more mutations (e.g., one or more conservative amino acid substitutions, as described herein). In yet another example, any nucleic acid fragment can include a stretch of at least about 5 (e.g., about 7, about 8, about 10, about 12, about 14, about 18, about 20, about 24, about 28, about 30, or more) nucleotides that are at least about 40% (about 50%, about 60%, about 70%, about 80%, about 90%, about 95%, about 87%, about 98%, about 99%, or about 100%) identical to any of the sequences described herein can be utilized in accordance with the invention.

The term “conservative amino acid substitution” refers to the interchangeability in proteins of amino acid residues having similar side chains (e.g., of similar size, charge, and/or polarity). For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamic acid and aspartic acid; and a group of amino acids having sulfur containing side chains consists of cysteine and methionine. Exemplary conservative amino acid substitution groups are valine-leucine-isoleucine, phenylalanine-tyrosine, lysine-arginine, alanine-valine, glycine-serine, glutamate-aspartate, and asparagine-glutamine.

As used herein, when a polypeptide or nucleic acid sequence is referred to as having “at least X % sequence identity” to a reference sequence, it is meant that at least X percent of the amino acids or nucleotides in the polypeptide or nucleic acid are identical to those of the reference sequence when the sequences are optimally aligned. An optimal alignment of sequences can be determined in various ways that are within the skill in the art, for instance, the Smith Waterman alignment algorithm (Smith T F et al., J. Mol. Biol. 1981; 147:195-7) and BLAST (Basic Local Alignment Search Tool; Altschul S F et al., J. Mol. Biol. 1990; 215:403-10). These and other alignment algorithms are accessible using publicly available computer software such as “Best Fit” (Smith T F et al., Adv. Appl. Math. 1981; 2(4):482-9) as incorporated into GeneMatcher Plus™ (Schwarz and Dayhof, “Atlas of Protein Sequence and Structure,” ed. Dayhoff, M. O., pp. 353-358, 1979). BLAST, BLAST-2, BLAST-P, BLAST-N. BLAST-X, WU-BLAST-2, ALIGN, ALIGN-2, CLUSTAL, T-COFFEE, MUSCLE, MAFFT, or Megalign (DNASTAR). In addition, those skilled in the art can determine appropriate parameters for measuring alignment, including any algorithms needed to achieve optimal alignment over the length of the sequences being compared. In general, for polypeptides, the length of comparison sequences can be at least five amino acids, preferably 10, 20, 30, 40, 50, 60, 70, 80, 90, 100, 125, 150, 175, 200, 250, 300, 400, 500, 600, 700, or more amino acids, up to the entire length of the polypeptide. For nucleic acids, the length of comparison sequences can generally be at least 10, 20, 30, 40, 50, 60, 70, 80, 90, 100, 125, 150, 175, 200, 250, 300, 400, 500, 600, 700, 800, 900, 1000, 1100, 1200, 1300, 1400, 1500, 1600, 1700, 1800, 1900, 2000, 2100, or more nucleotides, up to the entire length of the nucleic acid molecule. It is understood that for the purposes of determining sequence identity when comparing a DNA sequence to an RNA sequence, a thymine nucleotide is equivalent to a uracil nucleotide.

By “substantial identity” or “substantially identical” is meant a polypeptide or nucleic acid sequence that has the same polypeptide or nucleic acid sequence, respectively, as a reference sequence, or has a specified percentage of amino acid residues or nucleotides, respectively, that are the same at the corresponding location within a reference sequence when the two sequences are optimally aligned. For example, an amino acid sequence that is “substantially identical” to a reference sequence has at least about 50%, 60%, 70%, 75%, 80%, 85%, 90%, 95%, 96%, 97%, 98%, 99%, or 100% sequence identity to the reference amino acid sequence. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids (e.g., a full-length sequence). For nucleic acids, the length of comparison sequences will generally be at least 5, 10, 11, 12, 13, 14, 15, 16, 17, 18, 19, 20, 21, 22, 23, 24, or 25 contiguous nucleotides (e.g., the full-length nucleotide sequence). Sequence identity may be measured using sequence analysis software on the default setting (e.g., Sequence Analysis Software Package of the Genetics Computer Group. University of Wisconsin Biotechnology Center, 1710 University Avenue, Madison, Wis., 53705). Such software may match similar sequences by assigning degrees of homology to various substitutions, deletions, and other modifications.

The term “chimeric” as used herein as applied to a nucleic acid or polypeptide refers to two components that are defined by structures derived from different sources. For example, where “chimeric” is used in the context of a chimeric polypeptide (e.g., a chimeric Cas9/Csn1 protein), the chimeric polypeptide includes amino acid sequences that are derived from different polypeptides. A chimeric polypeptide may comprise either modified or naturally-occurring polypeptide sequences (e.g., a first amino acid sequence from a modified or unmodified Cas9/Csn1 protein; and a second amino acid sequence other than the Cas9/Csn1 protein). Similarly, “chimeric” in the context of a polynucleotide encoding a chimeric polypeptide includes nucleotide sequences derived from different coding regions (e.g., a first nucleotide sequence encoding a modified or unmodified Cas9/Csn1 protein; and a second nucleotide sequence encoding a polypeptide other than a Cas9/Csn1 protein).

The term “chimeric polypeptide” refers to a polypeptide which is made by the combination (i.e., “fusion”) of two otherwise separated segments of amino sequence, usually through human intervention. A polypeptide that comprises a chimeric amino acid sequence is a chimeric polypeptide. Some chimeric polypeptides can be referred to as “fusion variants.”

“Heterologous,” as used herein, means a nucleotide or polypeptide sequence that is not found in the native nucleic acid or protein, respectively. For example, in a chimeric Cas9/Csn1 protein, the RNA-binding domain of a naturally-occurring bacterial Cas9/Csn1 polypeptide (or a variant thereof) may be fused to a heterologous polypeptide sequence (i.e., a polypeptide sequence from a protein other than Cas9/Csn1 or a polypeptide sequence from another organism). The heterologous polypeptide sequence may exhibit an activity (e.g., enzymatic activity) that will also be exhibited by the chimeric Cas9/Csn1 protein (e.g., methyltransferase activity, acetyltransferase activity, kinase activity, ubiquitinating activity, etc.). A heterologous nucleic acid sequence may be linked to a naturally-occurring nucleic acid sequence (or a variant thereof) (e.g., by genetic engineering) to generate a chimeric nucleotide sequence encoding a chimeric polypeptide. As another example, in a fusion variant Cas9 site-directed polypeptide, a variant Cas9 site-directed polypeptide may be fused to a heterologous polypeptide (i.e., a polypeptide other than Cas9), which exhibits an activity that will also be exhibited by the fusion variant Cas9 site-directed polypeptide. A heterologous nucleic acid sequence may be linked to a variant Cas9 site-directed polypeptide (e.g., by genetic engineering) to generate a nucleotide sequence encoding a fusion variant Cas9 site-directed polypeptide.

“Recombinant,” as used herein, means that a particular nucleic acid, as defined herein, is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below). Alternatively, DNA sequences encoding RNA (e.g., DNA-targeting RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynuclcotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may be a naturally occurring amino acid sequence.

A “target sequence” as used herein is a polynucleotide (e.g., as defined herein, including a DNA, RNA, or DNA/RNA hybrid, as well as modified forms thereof) that includes a “target site.” The terms “target site” or “target protospacer DNA” are used interchangeably herein to refer to a nucleic acid sequence present in a target genomic sequence (e.g., DNA or RNA in a host cell) to which a targeting portion of the guiding component will bind provided sufficient conditions (e.g., sufficient complementarity) for binding exist. Suitable DNA/RNA binding conditions include physiological conditions normally present in a cell. Other suitable DNA/RNA binding conditions (e.g., conditions in a cell-free system) are known in the art; see, e.g., Sambrook, supra.

By “cleavage” it is meant the breakage of the covalent backbone of a target sequence (e.g., a nucleic acid molecule). Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, a complex comprising a guiding component and a nuclease is used for targeted double-stranded DNA cleavage. In other embodiments, a complex comprising a guiding component and a nuclease is used for targeted single-stranded RNA cleavage.

“Nuclease” and “endonuclease” are used interchangeably herein to mean an enzyme which possesses catalytic activity for DNA cleavage and/or RNA cleavage.

By “cleavage domain” or “active domain” or “nuclease domain” of a nuclease it is meant the polypeptide sequence or domain within the nuclease which possesses the catalytic activity for nucleic acid cleavage. A cleavage domain can be contained in a single polypeptide chain or cleavage activity can result from the association of two (or more) polypeptides. A single nuclease domain may consist of more than one isolated stretch of amino acids within a given polypeptide.

A “host cell,” as used herein, denotes an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells can be, or have been, used as recipients for a nucleic acid, and include the progeny of the original cell which has been transformed by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a subject bacterial host cell is a genetically modified bacterial host cell by virtue of introduction into a suitable bacterial host cell of an exogenous nucleic acid (e.g., a plasmid or recombinant expression vector) and a subject eukaryotic host cell is a genetically modified eukaryotic host cell (e.g., a mammalian germ cell), by virtue of introduction into a suitable eukaryotic host cell of an exogenous nucleic acid.

By “linker” is meant any useful multivalent (e.g., bivalent) component useful for joining to different portions or segments. Exemplary linkers include a nucleic acid sequence, a chemical linker, etc. In one instance, the linker of the guiding component (e.g., linker L in the interacting portion of the guiding component) can have a length of from about 3 nucleotides to about 100 nucleotides. For example, the linker can have a length of from about 3 nucleotides (nt) to about 90 nt, from about 3 nucleotides (nt) to about 80 nt, from about 3 nucleotides (nt) to about 70 nt, from about 3 nucleotides (nt) to about 60 nt, from about 3 nucleotides (nt) to about 50 nt, from about 3 nucleotides (nt) to about 40 nt, from about 3 nucleotides (nt) to about 30 nt, from about 3 nucleotides (nt) to about 20 nt or from about 3 nucleotides (nt) to about 10 nt. For example, the linker can have a length of from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. In some embodiments, the linker of a single-molecule guiding component is 4 nt.

The term “histone-packaged supercoiled plasmid DNA” is used to describe a component of protocells or carriers according to the present invention which utilize a plasmid DNA which has been “supercoiled” (i.e., folded in on itself using a supersaturated salt solution or other ionic solution which causes the plasmid to fold in on itself and “supercoil” in order to become more dense for efficient packaging into the protocells or carriers). The plasmid may be virtually any plasmid that expresses any number of polypeptides or encode RNA, including small hairpin RNA/shRNA or small interfering RNA/siRNA, as otherwise described herein. Once supercoiled (using the concentrated salt or other anionic solution), the supercoiled plasmid DNA is then complexed with histone proteins to produce a histone-packaged “complexed” supercoiled plasmid DNA.

“Packaged” DNA herein refers to DNA that is loaded into protocells or carriers (either adsorbed into the pores, confined directly within the nanoporous silica core itself, or encapsulated as a biological package). To minimize the DNA spatially, it is often packaged, which can be accomplished in several different ways, from adjusting the charge of the surrounding medium to creation of small complexes of the DNA with, for example, lipids, proteins, or other nanoparticles (usually, although not exclusively cationic). Packaged DNA is often achieved via lipoplexes (i.e., complexing DNA with cationic lipid mixtures). In addition, DNA has also been packaged with cationic proteins (including proteins other than histones), as well as gold nanoparticles (e.g., NanoFlares-an engineered DNA and metal complex in which the core of the nanoparticle is gold).

Any number of histone proteins, as well as other means to package the DNA into a smaller volume such as normally cationic nanoparticles, lipids, or proteins, may be used to package the supercoiled plasmid DNA “histone-packaged supercoiled plasmid DNA.” In certain aspects of the invention, a combination of histone proteins H1, H2A, H2B, H3 and H4 in a preferred ratio of 1:2:2:2:2, although other histone proteins may be used in other, similar ratios, as is known in the art or may be readily practiced pursuant to the teachings of the present invention. The DNA may also be double stranded linear DNA, instead of plasmid DNA, which also may be optionally supercoiled and/or packaged with histones or other packaging components.

Other histone proteins which may be used in this aspect of the invention include, for example, H1F, H1A, H1B, H2A, H2B, H1F0, H1FNT, H1FOO, H1FX, H1H1, HIST1H1A, HIST1H1B, HIST1H1C, HIST1H1D, HIST1H1E, HIST1H1T; H2AF, H2AFB1, H2AFB2, H2AFB3, H2AFJ, H2AFV, H2AFX, H2AFY, H2AFY2, H2AFZ, H2A1, HIST1H2AA, HIST1H2AB, HIST1H2AC, HIST1H2AD, HIST1H2AE, HIST1H2AG, HIST1H2AI, HIST1H2AJ, HIST1H2AK, HIST1H2AL, HIST1H2AM, H2A2, HIST2H2AA3, HIST2H2AC, H2BF, H2BFM, HSBFS, HSBFWT, H2B1, HIST1H2BA, HIST1HSBB, HIST1HSBC, HIST1HSBD, HIST1H2BE, HIST1H2BF, HIST1H2BG, HIST1H2BH, HIST1H2BI, HIST1H2BJ, HIST1H2BK, HIST1H2BL, HIST1H2BM, HIST1H2BN, HIST1H2BO, H2B2, HIST2H2BE, H3A1, HIST1H3A, HIST1H3B, HIST1H3C, HIST1H3D, HIST1H3E, HIST1H3F, HIST1H3G, HIST1H3H, HIST1H3I, HIST1H3J, H3A2, HIST2H3C, H3A3, HIST3H3, H41, HIST1H4A, HIST1H4B, HIST1H4C, HIST1H4D, HIST1H4E, HIST1H4F, HIST1H4G, HIST1H4H, HIST1H4I, HIST1H4J, HIST1H4K, HIST1H4L, H44, and HIST4H4.

The term “nuclear localization sequence” refers to a peptide sequence incorporated or otherwise crosslinked into histone proteins, which comprise the histone-packaged supercoiled plasmid DNA. In certain embodiments, protocells or carriers according to the present invention may further comprise a plasmid (often a histone-packaged supercoiled plasmid DNA) which is modified (crosslinked) with a nuclear localization sequence (note that the histone proteins may be crosslinked with the nuclear localization sequence or the plasmid itself can be modified to express a nuclear localization sequence), which enhances the ability of the histone-packaged plasmid to penetrate the nucleus of a cell and deposit its contents there (to facilitate expression and ultimately cell death. These peptide sequences assist in carrying the histone-packaged plasmid DNA and the associated histones into the nucleus of a targeted cell, whereupon the plasmid will express peptides and/or nucleotides as desired to deliver therapeutic and/or diagnostic molecules (polypeptide and/or nucleotide) into the nucleus of the targeted cell. Any number of crosslinking agents, well known in the art, may be used to covalently link a nuclear localization sequence to a histone protein (often at a lysine group or other group which has a nucleophilic or electrophilic group in the side chain of the amino acid exposed pendant to the polypeptide), which can be used to introduce the histone packaged plasmid into the nucleus of a cell. Alternatively, a nucleotide sequence that expresses the nuclear localization sequence can be positioned in a plasmid in proximity to that which expresses histone protein, such that the expression of the histone protein conjugated to the nuclear localization sequence will occur thus facilitating transfer of a plasmid into the nucleus of a targeted cell.

The terms “nucleic acid regulatory sequences.” “control elements,” and “regulatory elements,” used interchangeably herein, refer to transcriptional and translational control sequences, such as promoters, enhancers, polyadenylation signals, internal ribosomal entry sites (IRES), terminators, protein degradation signals, and the like, that provide for and/or regulate transcription of a non-coding sequence (e.g., DNA-targeting RNA) or a coding sequence (e.g., site-directed modifying polypeptide, or Cas9/Csn1 polypeptide) and/or regulate translation of an encoded polypeptide.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation, as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence. Transcriptional and translational control sequences are DNA regulatory sequences, such as promoters, enhancers, polyadenylation signals, terminators, and the like, that provide for the expression of a coding sequence in a host cell.

A “vector” or “expression vector” is a replicon, such as plasmid, phage, virus, or cosmid, to which another nucleic acid segment, i.e., an “insert”, may be attached so as to bring about the replication of the attached segment in a cell.

An “expression cassette” comprises a nucleic acid coding sequence operably linked, as defined herein, to a promoter sequence, as defined herein.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

“Operably linked” or “operatively linked” or “operatively associated with,” as used interchangeably, refers to a juxtaposition wherein the components so described are in a relationship permitting them to function in their intended manner. For instance, a promoter is operably linked to a coding sequence if the promoter affects its transcription or expression. A nucleic acid molecule is operatively linked or operably linked to, or operably associated with, an expression control sequence when the expression control sequence controls and regulates the transcription and translation of nucleic acid sequence. The term “operatively linked” includes having an appropriate start signal (e.g., ATG) in front of the nucleic acid sequence to be expressed and maintaining the correct reading frame to permit expression of the nucleic acid sequence under the control of the expression control sequence and production of the desired product encoded by the nucleic acid sequence. If a gene that one desires to insert into a recombinant DNA molecule does not contain an appropriate start signal, such a start signal can be inserted in front of the gene.

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-Ill; Celis, ed., 1994, “Cell Biology: A Laboratory Handbook” Volumes I-Ill; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL.

Other features and advantages of the invention will be apparent from the following description and the claims.

### BRIEF DESCRIPTION OF THE DRAWINGS

FIG. 1A-1D shows exemplary particles configured as silica carriers for use in a host cell (e.g., present in a plant or an alga). Provided are (A) a silica carrier 105 formed around a biological package 101 having a dimension d_{b} and (B) a silica carrier 1005 formed around a biological package 1001 and further including one or more cargos 1006. (C) Also provided is a schematic depicting use of a silica carrier as a NanoCRISPR platform to deliver CRISPR components in a targeted manner. The left half of the schematic depicts the NanoCRISPR(s) containing a plasmid as the biological package, and the right half depicts the NanoCRISPR(s) containing a phage as the biological package. NanoCRISPRs can include a therapeutic biological package (e.g., plasmids that encode Cas/guiding components that target the plant or alga genome; and phages that infect bacteria that may be present in plant or alga and encode Cas/guiding components that target essential bacterial genes in the bacterial DNA genome) coated with a shell of amorphous silica to stabilize the therapeutic, upon room-temperature storage and during delivery, and control its rate of release inside of target host cells. The silica surface can be optionally modified with biocompatible lipids to increase the colloidal stability of NanoCRISPRs and to facilitate their conjugation with ligands that target particular cells or organelles within the host cell or that promote endosomal escape of NanoCRISPRs upon host cell uptake. (D) Also shown is a schematic of a NanoCRISPR delivery platform (e.g., a protocell or a silica carrier) interacting with the host cell to deliver the biological package. (1) Targeting ligands conjugated to the NanoCRISPR surface can bind to corresponding receptors on the host cell. (2) Binding can trigger receptor-mediated endocytosis of NanoCRISPRs. (3) Endosomes become acidified, which will cause the lipid coating to dissociate from the NanoCRISPR's silica surface. (4) Endosome acidification will also protonate endosomolytic peptides, which will rupture endosomes via the proton-sponge mechanism. (5) Once in the cell's cytosol, the NanoCRISPR's silica shell will dissolve via hydrolysis, thereby releasing encapsulated CRISPR/Cas9 constructs (plasmids, in this case) and allowing them to act on their target RNA or DNA sequence.

FIG. 2A-2B shows exemplary methods for genome editing in the host cell. Provided are schematics for (A) a one-step process and (B) a two-step process employing particle-mediated CRISPR-Cas9 genome editing. In (A), the delivery platform includes a biological package having both a guiding component (e.g., gRNA) and a nuclease (e.g., Cas9). In (B), the first delivery platform includes a biological package having a nuclease (e.g., Cas9), and the second delivery platform includes a biological package having a guiding component (e.g., gRNA).

FIG. 3A-3B shows exemplary particles configured as protocells for use in a host cell (e.g., present in a plant or an alga). Provided are (A) a protocell 205 having a porous core 201 having a dimension d_{core} and a dimension dₚₒᵣₑ and (B) a schematic depicting use of a protocell as a NanoCRISPR platform for highly efficacious delivery of CRISPR-based components. Host-directed CRISPR components (e.g., guide components, such as guide RNAs, as well as minicircle DNA vectors that encode Cas and guiding components) will be developed, along with strategies for introducing CRISPR components into plants or algae. Non-limiting strategies include modifying CRISPR components will cell-penetrating peptides, co-delivering CRISPR components with metal organic frameworks (MOFs), and/or developing phage that encode CRISPR components. CRISPR components can be loaded within mesoporous silica nanoparticles (MSNPs) and/or encased in a supported lipid bilayer (SLB). Resulting NanoCRISPRs can be optionally surface-modified with molecules that promote their accumulation within targeted organelles and trigger their uptake by host cells.

FIG. 4A-4C shows (A) Nannochloropsis salina, (B) Chlorella variabilis, and (C) Haematococcus pluvialis imaged with nanoparticles loaded with fluorescent dye. Left images are compressed and overlaid brightfield, GFP fluorescence, and Ch1 fluorescence images; while right images are compressed and overlaid GFP and Ch1 fluorescence images. Nanoparticle fluorescence was green while chlorophyll fluorescence was red. Arrows indicate potential evidence of nanoparticle uptake. Nanoparticles were (A) PF 2.1, (B) PF 2.3, (C, right) PF 1.1, and (C, left) PF 2.2, as described in Table 2.

FIG. 5A-5E shows non-limiting, proposed genetic modifications for an alga host cell. Provided are schematics of (A) dark respiration and (B) photorespiration pathways in algae with proposed genetic modifications marked by **. Abbreviations include the following: AOX—alternative oxidase, ATP—adenosine triphosphate, CoA—coenzyme A, FADH₂—flavin adenine dinucleotide. FFA—free fatty acid, GCL—glycolate carboxyligase, GDH—glycolate dehydrogenase, NADH—nicotinamide adenine dinucleotide, NH₃—ammonia, RuBP—ribulose-1,5-bisphosphate, TAG—triacylglycerol, TCA—tricarboxylic acid, and TSR—tartronic semialdehyde reductase. Also provided are amino acid sequences for potential targets in N. gaditana, including sequences for (C) laminarinase (SEQ ID NO:201), TAG lipase CrLIP1 (SEQ ID NO:202), and TAG lipase SDP1 (SEQ ID NO:203); (D) cytochrome c oxidase (SEQ ID NOs:204-205) and alternative oxidase (SEQ ID NO:206); and (E) glycolate dehydrogenase (SEQ ID NO:207), glycolate carboxyligase (SEQ ID NO:208), and tartronic semialdehyde reductase (SEQ ID NO:209).

FIG. 6A-6C shows a CRISPR component and its non-limiting use with a delivery platform described herein. (A) CRISPR naturally evolved in prokaryotes as a type of acquired immune system, conferring resistance to exogenous genetic sequences introduced by plasmids and phages. The CRISPR array is a noncoding RNA transcript, and the CRISPR repeat arrays are often associated with Cas (i.e., ‘CRISPR-associated’) protein families. Exogenous DNA is cleaved by Cas proteins into ˜30-bp fragments, which are then inserted into the CRISPR locus (see (1) Acquisition in FIG. 6A). RNAs from the CRISPR loci are constitutively expressed (see (2) Expression in FIG. 6A) and direct other Cas proteins to cleave exogenous genetic elements upon subsequent exposure or infection (see (3) Interference in FIG. 6B). Cas9 is a RNA-Guided Endonuclease (R-GEN) adapted from the prokaryotic CRISPR system and is used by researchers as a novel, programmable tool for genome editing. Cas9 forms a sequence-specific endonuclease when complexed with a guide RNA that is complementary to the target sequence. (C) An exemplary CRISPR component includes a guiding component 90 to bind to the target sequence 97, as well as a nuclease 98 (e.g., a Cas nuclease or an endonuclease, such as a Cas endonuclease) that interacts with the guiding component and the target sequence.

FIG. 7A-7C shows non-limiting CRISPR components. Provided are schematics of (A) a non-limiting guiding component 300 having a targeting portion 304, a first portion 301, a second portion 302, and a linker 303 disposed between the first and second portions; (B) another non-limiting guiding component 350 having a targeting portion 354, a first portion 351, a second portion 352 having a hairpin, and a linker 353 disposed between the first and second portions; and (C) non-limiting interactions between the guiding component 400, the genomic sequence 412, and the first and second portion 401,402. As can be seen, the target sequence 411 of the genomic sequence 412 is targeted by way of non-covalent binding 421 to the targeting portion 404, and secondary structure can be optionally implemented by way of non-covalent binding 422 between the first portion 401 and the second portion 402. The targeting portion 404, first portion 401, linker 403, and second portion 402 can be attached in any useful manner (e.g., to provide a 5′ end 405 and a 3′ end 406).

FIG. 8A-8H shows non-limiting amino acid sequences for various nucleases. Provided are sequences for (A) a Cas9 endonuclease for S. pyogenes serotype M1 (SEQ ID NO:110), (B) a deactivated Cas9 having D10A and H840A mutations (SEQ ID NO: 111), (C) a Cas protein Csn1 for S. pyogenes (SEQ ID NO: 112), (D) a Cas9 endonuclease for F. novicida U112 (SEQ ID NO: 113), (E) a Cas9 endonuclease for S. thermophilus 1 (SEQ ID NO: 114), (F) a Cas9 endonuclease for S. thermophilus 2 (SEQ ID NO: 115), (G) a Cas9 endonuclease for L. innocua (SEQ ID NO: 116), and (H) a Cas9 endonuclease for W. succinogenes (SEQ ID NO: 117).

FIG. 9 shows non-limiting nucleic acid sequences of crRNA that can be employed as a first portion in any guiding component described herein. Provided are sequences for S. pyogenes (SEQ ID NO:20), L. innocua (SEQ ID NO:21), S. thermophilus 1 (SEQ ID NO:22), S. thermophilus 2 (SEQ ID NO:23), F. novicida (SEQ ID NO:24), and W. succinogenes (SEQ ID NO:25). Also provided are various consensus sequences (SEQ ID NOs:26-32), in which each X, independently, can be absent, A, C, T, G, or U, as well as modified forms thereof (e.g., as described herein). In another embodiment, for each consensus sequence (SEQ ID NOs:26-32), each X at each position is a nucleic acid (or a modified form thereof) that is provided in an aligned reference sequence. For instance, for consensus SEQ ID NO:26, the first position includes an X, and this X can be absent or any nucleic acid (e.g., A, C, T, G, or U, as well as modified forms thereof). Alternatively, this X can be any nucleic acid provided in an aligned reference sequence (e.g., aligned reference sequences SEQ ID NO:20-25 for the consensus sequence in SEQ ID NO:26). Thus, X at position 1 in SEQ ID NO:26 can also be G (as in SEQ ID NOs:20-23 and 25) or C (as in SEQ ID NO:24), in which this subset of substitutions is defined as a conservative subset. Similarly, for each X at each position for the consensus sequences (SEQ ID NOs:26-32), conservative subsets can be determined based on FIG. 9, and these consensus sequences include nucleic acid sequences encompassed by such conservative subsets. Gray highlight indicates a conserved nucleic acid, and the dash indicates an absent nucleic acid.

FIG. 10A-10C shows non-limiting nucleic acid sequences of tracrRNA that can be employed as a second portion and/or linker in any guiding component described herein. Provided are sequences for S. pyogenes (SEQ ID NO:40), L. innocua (SEQ ID NO:41), S. thermophilus 1 (SEQ ID NO:42), S. thermophilus 2 (SEQ ID NO:43), F. novicida 1 (SEQ ID NO:44), F. novicida 2 (SEQ ID NO:45), W. succinogenes 1 (SEQ ID NO:46), and W. succinogenes 2 (SEQ ID NO:47). Also provided are various consensus sequences (SEQ ID NOs:48-54), in which each Z, independently, can be absent. A, C, T, G, or U, as well as modified forms thereof (e.g., as described herein). Consensus sequences are shown for (A) an alignment of all SEQ ID NOs:40-47, providing consensus sequences SEQ ID NOs:48-50; (B) an alignment of SEQ ID NOs:40-43, providing consensus sequences SEQ ID NOs:51-52; and (C) an alignment of SEQ ID NOs:44-47, providing consensus sequences SEQ ID NOs:53-54. In another embodiment, for each consensus sequence (SEQ ID NOs:48-54), each Z at each position is a nucleic acid (or a modified form thereof) that is provided in an aligned reference sequence. For instance, for consensus SEQ ID NO:48, the first position includes a Z, and this Z can be absent or any nucleic acid (e.g., A, C, T, G, or U, as well as modified forms thereof). Alternatively, this Z can be any nucleic acid provided in an aligned reference sequence (e.g., aligned reference sequences SEQ ID NO:40-47 for the consensus sequence in SEQ ID NO:48). Thus, Z at position 2 in SEQ ID NO:48 can also be U (as in SEQ ID NOs:40, 41, and 43-47) or G (as in SEQ ID NO:42), in which this subset of substitutions is defined as a conservative subset. Similarly, for each Z at each position for the consensus sequences (SEQ ID NOs:48-54), conservative subsets can be determined based on FIG. 10A-10C, and these consensus sequences include nucleic acid sequences encompassed by such conservative subsets. Gray highlight indicates a conserved nucleic acid, and the dash indicates an absent nucleic acid.

FIG. 11 shows non-limiting nucleic acid sequences of extended tracrRNA that can be employed as a second portion and/or linker in any guiding component described herein. Provided are sequences for S. pyogenes (SEQ ID NO:60), L. innocua (SEQ ID NO:61), S. thermophilus 1 (SEQ ID NO:62), and S. thermophilus 2 (SEQ ID NO:63). Also provided are various consensus sequences (SEQ ID NOs:64-65), in which each Z, independently, can be absent, A, C, T, G, or U, as well as modified forms thereof (e.g., as described herein). In another embodiment, for each consensus sequence (SEQ ID NOs:64-65), each Z at each position is a nucleic acid (or a modified form thereof) that is provided in an aligned reference sequence. For instance, for consensus SEQ ID NO:64, the first position includes a Z, and this Z can be absent or any nucleic acid (e.g., A, C, T, G, or U, as well as modified forms thereof). Alternatively, this Z can be any nucleic acid provided in an aligned reference sequence (e.g., aligned reference sequences SEQ ID NO:60-63 for the consensus sequence in SEQ ID NO:64). Thus, Z at position 1 in SEQ ID NO:64 can also be absent (as in SEQ ID NO:60), A (as in SEQ ID NO:61), or U (as in SEQ ID NOs:63-64), in which this subset of substitutions is defined as a conservative subset. Similarly, for each Z at each position for the consensus sequences (SEQ ID NOs:64-65), conservative subsets can be determined based on FIG. 11, and these consensus sequences include nucleic acid sequences encompassed by such conservative subsets. Gray highlight indicates a conserved nucleic acid, and the dash indicates an absent nucleic acid.

FIG. 12 shows non-limiting nucleic acid sequences of a guiding component (e.g., a synthetic, non-naturally occurring guiding component) having a generic structure of A-L-B, in which A includes a first portion (e.g., any one of SEQ ID NOs:20-32, or a fragment thereof), L is a linker (e.g., a covalent bond, a nucleic acid sequence, a fragment of any one of SEQ ID NOs:40-54 and 60-65, or any other useful linker), and B is a second portion (e.g., any one of SEQ ID NOs:40-54 and 60-65, or a fragment thereof). Also provided are various embodiments of single-stranded guiding components (SEQ ID NOs:80-93). Exemplary non-limiting guiding components include SEQ ID NO:81, or a fragment thereof, where X at each position is defined as in SEQ ID NO:26 and Z at each position is as defined in SEQ ID NO:48; SEQ ID NO:82, or a fragment thereof, where X at each position is defined as in SEQ ID NO:27 and Z at each position is as defined in SEQ ID NO:49; SEQ ID NO:83, where X at each position is defined as in SEQ ID NO:28 and Z at each position is as defined in SEQ ID NO:49; SEQ ID NO:84, or a fragment thereof, where X at each position is defined as in SEQ ID NO:27 and Z at each position is as defined in SEQ ID NO:65; SEQ ID NO:85, or a fragment thereof, where X at each position is defined as in SEQ ID NO:28 and Z at each position is as defined in SEQ ID NO:65; SEQ ID NO:86, or a fragment thereof, where X at each position is defined as in SEQ ID NO:29 and Z at each position is defined as in SEQ ID NO:51; SEQ ID NO:87, or a fragment thereof, where X at each position is defined as in SEQ ID NO:30 and Z at each position is defined as in SEQ ID NO:51; SEQ ID NO:88, or a fragment thereof, where X at each position is defined as in SEQ ID NO:30 and Z at each position is defined as in SEQ ID NO:52; SEQ ID NO:89, or a fragment thereof, where X at each position is defined as in SEQ ID NO:30 and Z at each position is defined as in SEQ ID NO:65; SEQ ID NO:90, or a fragment thereof, where X at each position is defined as in SEQ ID NO:31 and Z at each position is defined as in SEQ ID NO:51; SEQ ID NO:91, or a fragment thereof, where X at each position is defined as in SEQ ID NO:32 and Z at each position is as defined in SEQ ID NO:53; SEQ ID NO:92, or a fragment thereof, where X at each position is defined as in SEQ ID NO:32 and Z at each position is as defined in SEQ ID NO:54; and SEQ ID NO:93, or a fragment thereof, where X at each position is defined as in SEQ ID NO:32 and Z at each position is defined as in SEQ ID NO:65. The fragment can include any useful number of nucleotides (e.g., any number of contiguous nucleotides, such as a fragment including about 4, 5, 6, 7, 8, 9, 10, 11, 12, 15, 18, 20, or more contiguous nucleotides of any sequences described herein, such as a sequence for the first portion, e.g., any one of SEQ ID NOs:20-32; and also such as a fragment including about 4, 5, 6, 7, 8, 9, 10, 11, 12, 15, 18, 20, 24, 26, 28, 30, 32, 34, 38, 36, 40, or more contiguous nucleotides of any sequences described herein, such as a sequence for the first portion, e.g., any one of SEQ ID NOs:40-54 and 60-65).

FIG. 13 shows additional non-limiting nucleic acid sequences of a guiding component (e.g., a synthetic, non-naturally occurring guiding component). Provided are various embodiments of single-stranded guiding components (SEQ ID NOs: 100-103). Exemplary non-limiting guiding components include SEQ ID NO: 100, or a fragment thereof, where n at each of positions 1-80 can be present or absent such that this region can contain anywhere from 12 to 80 nucleotides and n is A, C, T, G, U, or modified forms thereof; and where n at each of positions 93-192 can be present or absent such that this region can contain anywhere from 3 to 100 nucleotides and n is A, C, T, G, U, or modified forms thereof; SEQ ID NO:101, or a fragment thereof, where n at each of positions 1-80 can be present or absent such that this region can contain anywhere from 12 to 80 nucleotides and n is A, C, T, G, U, or modified forms thereof; and where n at each of positions 93-192 can be present or absent such that this region can contain anywhere from 3 to 100 nucleotides and n is A, C, T, G, U, or modified forms thereof; SEQ ID NO: 102, or a fragment thereof, where n at each of positions 1-80 can be present or absent such that this region can contain anywhere from 12 to 80 nucleotides and n is A, C, T, G, U, or modified forms thereof; and SEQ ID NO: 103, or a fragment thereof, where n at each of positions 1-80 can be present or absent such that this region can contain anywhere from 12 to 80 nucleotides and n is A, C, T, G, U, or modified forms thereof.

FIG. 14A-14B shows an aerosol-assisted EISA for a rapid, cost-effective, scalable method for producing MSNPs with reproducible properties. Provided are (A) a non-limiting schematic and (B) a photograph of an exemplary reactor to generate MSNPs, protocells, and/or carriers via aerosol-assisted EISA. Numbers indicate corresponding portions of the reactor.

FIG. 15 shows that aerosol-assisted EISA can be used to generate MSNPs with various pore geometries. TEM images of MSNPs with hexagonal (A), cubic (B), lamellar (C), and cellular (D-E) pore geometries (F) shows dual-templated particles with interconnected 2 nm and 60 nm pores. Light grey/white areas are voids (i.e., pores), while dark grey/black areas are silica.

FIG. 16 shows that aerosol-assisted EISA can be used to generate MSNPs with various pore sizes. TEM images of MSNPs with 2.5 nm pores templated by CTAB (A), 4.4 nm pores templated by F68 (B), 7.9 nm pores templated by F127 (C), and 18-25 nm pores templated by crosslinked micelles (D). The inset in (D) is a SEM micrograph that shows the presence of surface-accessible pores.

FIG. 17 shows that lipid coated silica (LCS) delivery platforms have extremely high loading capacities for various small molecules (e.g., antibiotics) having varying molecular weights and net charges at physiological pH. Data represent the mean+std. dev. for n=3.

FIG. 18A-18D shows the degree of condensation of the MSNP core, which can be used to tailor release rates from burst to sustained profiles. Rates of gentamicin release from MSNPS with a low (A) and high (B) degree of silica condensation. Silica forms via a condensation reaction (C) and dissolves via a hydrolysis reaction (D); the degree of silica condensation dictates that number of Si—O—Si bonds that must be broken for the particle to dissolve and can, therefore, be used to control release rates. Data represent the mean±std. dev. for n=3.

FIG. 19A-19B shows that LCS delivery platforms are selectively internalized by model Bp host cells when modified with cell-specific targeting ligands. (A) The number of LCS particles internalized by THP-1 (model macrophage), A549 (model alveolar epithelial cell), and HepG2 (model hepatocyte) cells upon incubation with a 10⁴-fold excess of LCS particles for 1 hour at 37° C. LCS particles were coated with DOPC (net neutral charge at physiological pH), DOPS (net negative charge), or DOTAP (net positive charge); DOPC LCS particles were further targeted to THP-1, A549, and HepG2 cells using a DEC-205 scFv, the GE11 peptide, and the SP94 peptide, respectively. Data represent the mean±std. dev. for n=3. (B) Confocal fluorescence microscopy images of THP-1, A549, and HepG2 cells after being incubated with a 10⁴-fold excess of LCS particles for 1 hour at 37° C. LCS particles were loaded with pHrodo Red (red), the fluorescence intensity of which dramatically increases under endolysosomal conditions, and labeled with NBD (green), the fluorescence intensity of which is independent of pH, and targeted to THP-1, A549, and HepG2 cells using a DEC-205 scFv, the GE11 peptide, and the SP94 peptide, respectively. Cell nuclei were stained with DAPI (blue).

FIG. 20A-20B shows that protocells have high capacities for physicochemically disparate medical countermeasures and controllable, pH-triggered release rates. (A) Loading capacities of 150 nm protocells with 2.5 nm pores, 4.4 nm pores, 7.9 nm pores, and 18-25 nm pores for different classes of small molecule (ribavirin, ceftazidime), protein (hPON-1, OPH, hBuChE), and nucleic acid (siRNA, mcDNA, pDNA)-based medical countermeasures (siRNA, mcDNA, pDNA); loading capacities of 150 nm liposomes are provided for comparison. Molecular weights (MW) and mean hydrodynamic sizes in 1×PBS are given for each cargo molecule. * indicates the hydrodynamic size of the pDNA after being packaged with histones. (B) Rates of ribavirin release from protocells with DOPC SLBs when incubated in a simulated body fluid (pH 7.4) or a simulated endolysosomal fluid (pH 5.0) at 37° C. for 7 days; the rate of ribavirin release from DSPC liposomes upon incubation in a simulated body fluid is given for comparison. Data represent the mean±std. dev. for n=3.

FIG. 21A-21B shows that LCS particles remain stable in blood, as evidenced by their near-constant sizes and surface charges. Mean hydrodynamic size (A) and zeta potential (B) of LCS particles, LCS particles modified with 10 wt % of PEG-2000, PEI-coated silica NPs, PEI-coated silica NPs modified with 10 wt % of PEG-2000 upon incubation in whole blood for 7 days at 37° C. Data represent the mean±std. dev. for n=3.

FIG. 22 shows that spray-drying LCS particles increases their room-temperature shelf-life. Time-dependent release of gentamicin from DOPC LCS particles that were stored in 1×PBS, as well as DOPC LCS particles that were spray-dried in the presence of trehalose or poly(lactide-co-glycolide) (PLGA) and stored in nitrogen-flushed septum vials. Data represent the mean±std. dev. for n=3.

FIG. 23A-23D shows that the supported lipid layers enabled pH-triggered release, where cargo molecules are retained in blood but released in a simulated endolysosomal fluid at various rates. (A),(C) TEM images of LCS particles with a 4 nm-thick supported lipid bilayer (SLB) (A) and a 11 nm-thick supported lipid multilayer (SLM) (C). (B),(D) Rates of gentamicin release from DOPC LCS particles when incubated in blood or a simulated endolysosomal fluid (SEF) at 37° C. for 14 days or 72 hours, respectively. LCS particles had a low or high degree of condensation (DOC). SLBs were either unmodified or modified to contain 5 wt % of a maleimide-containing lipid (MPB) that forms disulfide bond-based crosslinks in the presence of DTT. SLMs were three layers thick. Data represent the mean±std. dev. for n=3.

FIG. 24A-24B shows eight-color confocal fluorescence microscopy images of cells incubated with a 10⁴-fold excess of LCS particles for (A) 1 hour or (B) 24 hours at 37° C. LCS particles were simultaneously loaded with a fluorescently-labeled model drug (panel labeled “Drug-NLS”), siRNA (panel labeled “siRNA-NLS”), protein (panel labeled “Protein”), and QD-conjugated minicircle DNA (panel labeled “QD-DNA”). The lipid (panel labeled “Lipid”) and silica (panel labeled “Silica”) components of the LCS particle were individually labeled as well. Cells were stained with CellTracker Violet BMQC and DAPI (panel labeled “Cytosol & Nucleus”).

FIG. 25 shows that LCS particles that are targeted to the lung preferentially accumulate in the lungs over the liver. Time-dependent concentrations (depicted as percent of the injected dose, or % ID) of silicon (from silica NPs) in the livers and lungs of BALB/c mice upon IV injection of 50 mg/kg of DOPC LCS particles or DOPC LCS particles modified with a peptide ‘zipcode’ that targets lung vasculature. LCS particles had a mean diameter of 70 nm with a 30-110 nm size distribution. Silicon concentrations were determined using ICP-MS. Error bars represent the mean±the standard deviation for 10 mice.

FIG. 26 shows that by varying size and surface modifications, LCS particles can be engineered to remain in circulation for long periods of time. Time-dependent concentrations (depicted as percent of the injected dose, or % ID) of silicon (from silica NPs) and rhodamine B (used as a surrogate drug) in the blood of BALB/c mice upon IV injection of 50 mg/kg of free rhodamine B or rhodamine B loaded in LCS particles. LCS particles had a mean diameter of 70 nm with a 30-110 nm size distribution and were modified with CD47, a protein expressed by red blood cells that innate immune cells recognize as ‘self’. Silicon and rhodamine B concentrations in whole blood were determined using ICP-OES and HPLC-FLD, respectively. Error bars represent the mean±the standard deviation for 5 mice.

FIG. 27A-27B shows that LCS particles are biodegradable. (A) Concentrations (depicted as percent of the injected dose, or % ID) of silicon (from silica NPs) in the urine and feces of BALB/c mice 1 hour, 24 hours, 48 hours, 72 hours, 7 days, and 14 days after IV injection of a 200 mg/kg dose of empty DOPC LCS particles (70 nm in diameter with 30-110 nm size distribution). Silicon was quantified using ICP-MS. Data represent the mean+std. dev. for 5 mice. ND=none detected. (B) TEM image of MSNPs that appeared in the urine of a BALB/c mouse 7 days after IV injection with a 200 mg/kg dose of DOPC LCS particles; largely intact MSNPs are visible, along with silica remnants.

FIG. 28 shows that LCS particles are non-immunogenic. Serum IgG and IgM titers induced upon SC immunization of C57B1/6 mice with three doses of LCS particles or albumin NPs that were targeted to hepatocytes with a peptide (‘SP94’) identified via phage display. Mice were immunized on days 0, 14, and 28 with 20 g of LCS particles or albumin NPs; serum was collected on day 56, and peptide-specific IgG and IgM titers were determined via end-point dilution ELISA. Data represent the mean+std. dev. for 3 mice.

FIG. 29A-29B shows that formulating a model phage, MS2, in silica carriers (e.g., single phage-in-silica nanoparticles or “SPS NPs”) increases its room-temperature shelf-life and decreases its immunogenicity. (A) Titers of a MS2 liquid stock, MS2 spray-dried in the presence of Brij 58 (2.5 μm mean diameter). MS2 spray-dried in the presence of sucrose (2.2 μm mean diameter), MS2-based SPS NPs that do not contain silica (93 nm mean diameter), MS2-based SPS NPs that do contain silica (55 nm mean diameter), and silica-containing SPS NPs that were further spray-dried in the presence of trehalose (2.5 μm mean diameter) upon storage for 6 months at ambient temperature and humidity. MS2 stored as a liquid stock loses 460 logs of activity per month. Spray-dried MS2 loses 19-26 logs of activity per month. SPS NPs formed without silica lose 5.9 logs of activity per month. SPS NPs formed with silica lose 0.37 logs of activity per month. Finally, spray-dried SPS NPs lose 0.21 logs of activity in six months. (B) Anti-MS2 serum IgG titers for free MS2, MS2 spray-dried (SD) in the presence of sucrose, and MS2-based SPS NPs that contain silica, Brij 58, and sucrose. C57B/6 mice were immunized SC with 20 μg of MS2 on days 0, 14, and 28; serum was collected on day 56, and MS2-specific IgG titers were determined via end-point dilution ELISA. Each circle represents the titer achieved in one of four mice per group; lines represent the average titer per group.

FIG. 30 shows that spray-drying of silica carriers (e.g., SPS NPs) results in inhalable dry powders that show promising lung deposition upon insufflator-based administration to mice. (A)-(D) Size (A) and morphology (B-D) of dry powder particles (2.5 μm mean diameter) obtained upon spray-drying SPS NPs (55 nm mean diameter) in the presence of lactose. Size was determined using optical particle spectrometry, and morphology was determined using SEM (B, C) and TEM (D); arrows in (C) and (D) point to SPS NPs. SPS NPs contained the model phage, MS2, and were coated with the zwitterionic lipid, DOPC, prior to spray-drying; MS2 was labeled with electron-dense Sulfo-NHS-Nanogold® prior to its incorporation in SPS NPs. (E)-(F) The trachea, right lung, and left lung from BALB/c mice 1 hour after receiving no treatment (E) or 50 mg/kg of fluorescently-labeled SPS NPs in 200 μL puffs via a PennCentury dry powder insufflator, model DP-4 (F). The scale in (F) has units of (p/sec/cm²/sr)/(W/cm²).

### DETAILED DESCRIPTION OF THE INVENTION

The present invention relates to a delivery platform for transforming a plant or an alga, e.g., by genetic modification by use of a CRISPR component. In particular, the particles (e.g., protocells or carriers of the invention) are highly flexible and modular. For instance, high concentrations of physiochemically-disparate molecules can be loaded into the protocells or carriers and their therapeutic and/or diagnostic agent release rates can be optimized without altering the protocell's or carrier's size, size distribution, stability, or synthesis strategy. Properties of the supported lipid bi- or multilayer, particle core, and particle shell can also be modulated independently, thereby optimizing properties as surface charge, colloidal stability, and targeting specificity independently from overall size, type of cargo(s), loading capacity, and release rate. Additional details follow.

Delivery Platforms

Modifying genomic sequences of plants and algae require a robust platform capable of entering the host cell. In particular, CRISPR can be used to develop host-directed countermeasures. CRISPR components can be packaged within state-of-the-art nanoparticle delivery platforms (e.g., protocells or silica carriers), which can be modulated to have useful particle property, including size and surface modifications, that promote delivery to specific targets (e.g., organelles, mitochondria, chloroplasts, nuclei, etc.), uptake by host cells, and release within appropriate intracellular locations (e.g., to achieve targeted cleavage, activation, or inactivation of host DNA).

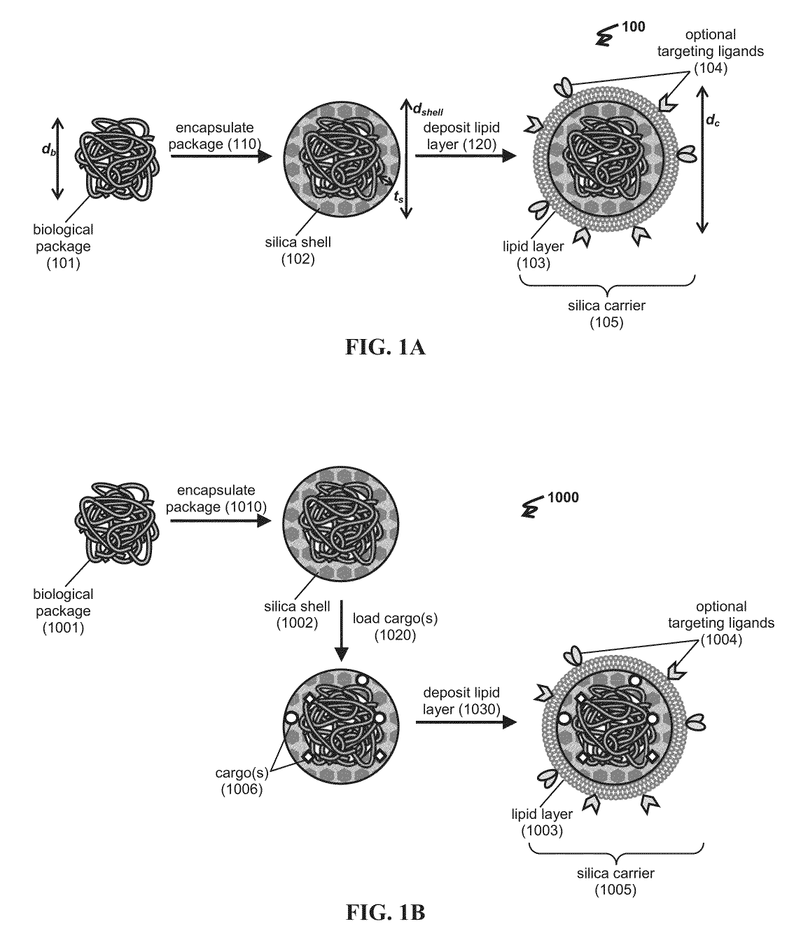

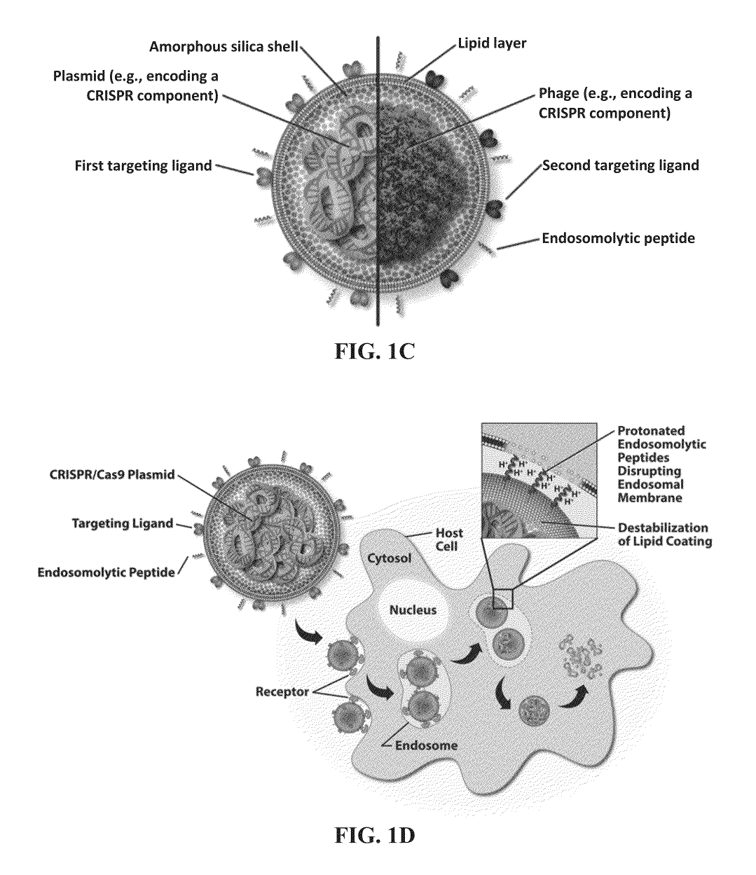

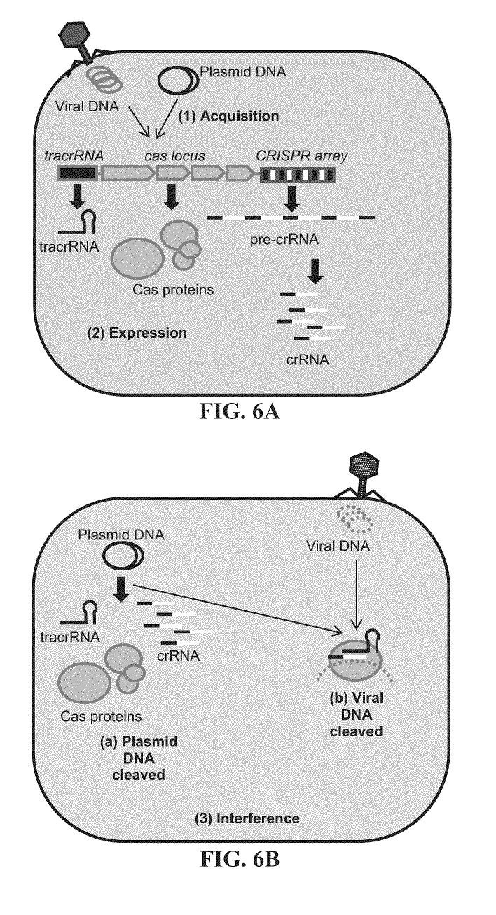

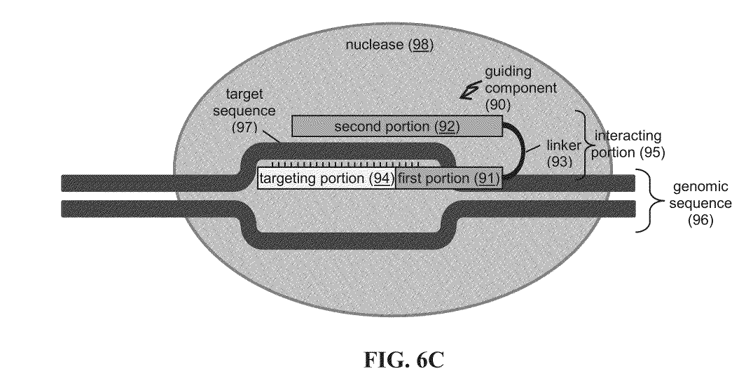

In one instance, the delivery platform includes a CRISPR component, such as a CRISPR/Cas system (e.g., a type I, II, or III CRISPR/Cas system, as well as modified versions thereof, such as a CRISPR/dCas9 system). Exemplary platforms are shown in FIGS. 1A-1D, 2A-2B, and 3A-3B, where exemplary CRISPR components are shown in FIGS. 6C, 7A-7C, 8A-8H, 9, 10A-10C, 11, 12, and 13.

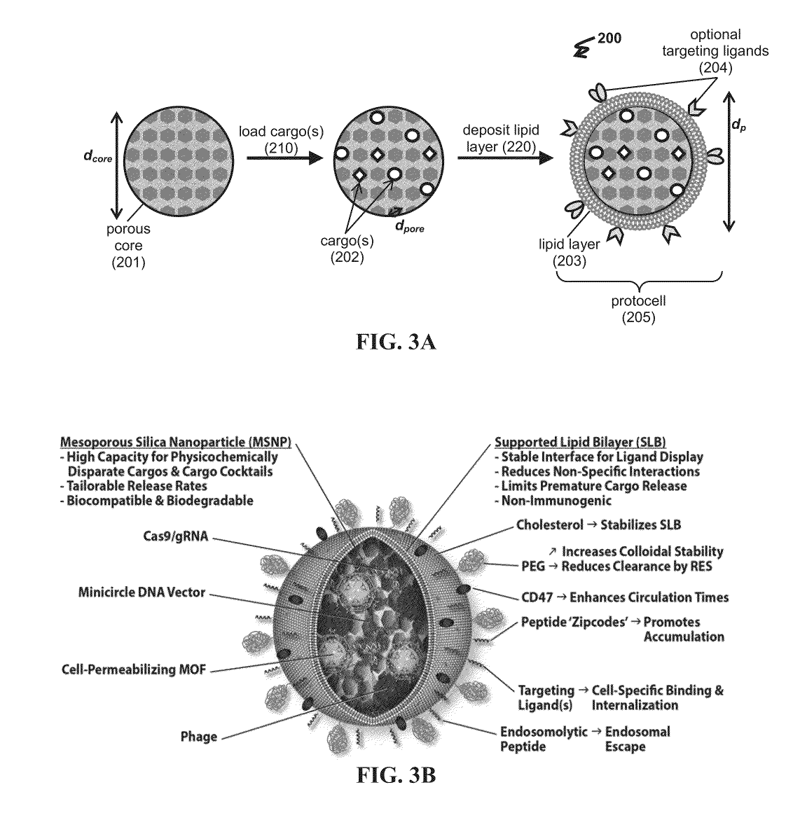

The delivery platform (e.g., a NanoCRISPR, as employed herein) can be based on a protocell (e.g., FIG. 3A-3B) or a carrier (e.g., FIG. 1A-1D). As described herein, the protocell includes a porous core (e.g., a porous silica core) having one or more cargo deposited within the plurality of pores of the core, whereas the carrier includes a shell (e.g., a silica shell) that encapsulates a biological package.

The silica carrier can be formed in any useful manner. As seen in the method 100 of FIG. 1A, a biological package 101 having a dimension d_{b} is first provided. Exemplary values for dimension d_{b} include, without limitation, greater than about 10 nm (e.g., greater than about 20 nm, 30 nm, 40 nm, 50 nm, 60 nm, 70 nm, 80 nm, 90 nm, 100 nm, 125 nm, 150 nm, 200 nm, 300 nm, 500 nm, 750 nm, 1 μm, 2 μm, 5 μm, 10 μm, 20 μm, or more). The biological package can include one or more components (e.g., one or more nucleic acid sequences, drugs, proteins, labels, etc., such as any agent described herein).

Then, the biological package 101 is encapsulated 110 with a silica shell 102 having a thickness tₛ, thereby providing a particle of dimension dₛₕₑₗₗ. The shell can have any useful thickness that allows for controlled biodegradation in vivo, targeted biodistribution, stability in a formulation, and/or consistent fabrication of the carrier (or a population of carriers). Exemplary values for dimension tₛ include, without limitation, less than about 100 nm (e.g., less than about 0.1 nm, 0.5 nm, 1 nm, 2 nm, 3 nm, 5 nm, 8 nm, 10 nm, 15 nm, 20 nm, 30 nm, 40 nm, 50 nm, 60 nm, 70 nm, 80 nm, 90 nm). Exemplary values for dimension dₛₕₑₗₗ include, without limitation, greater than about 10 nm (e.g., greater than about 20 nm, 30 nm, 40 nm, 50 nm, 60 nm, 70 nm, 80 nm, 90 nm, 100 nm, 125 nm, 150 nm, 200 nm, 300 nm, 500 nm, 750 nm, 1 μm, 2 μm, 5 μm, 10 μm, 20 μm, or more).

Finally, an optional lipid layer 103 can be deposited 120 on an outer surface of the silica shell (e.g., thereby forming a silica carrier 105). Furthermore, one or more optional targeting ligands 104 (e.g., any described herein) can be combined and/or co-extruded with the lipid and then deposited as a lipid layer (e.g., a lipid bilayer or a lipid multilayer). The silica carrier 105 can have any useful dimension d_{c}. Exemplary values for dimension d_{c} include, without limitation, greater than about 10 nm (e.g., greater than about 20 nm, 30 nm, 40 nm, 50 nm, 60 nm, 70 nm, 80 nm, 90 nm, 100 nm, 125 nm, 150 nm, 200 nm, 300 nm, 500 nm, 750 nm, 1 μm, 2 μm, 5 μm, 10 μm, 20 μm, or more).

Optionally, the method can be adapted to include any other useful component(s) or cargo(s). As seen in the method 1000 of FIG. 1B, a biological package 1001 is encapsulated 1010 with a silica shell 1002. One or more cargos 1006 can be loaded 1020 into the shell (if the shell is porous) or onto the outer surface of the shell (e.g., if the shell is not porous). A lipid layer 1003 can be deposited 1030 on an outer surface of the silica shell (e.g., thereby forming a silica carrier 1005). Furthermore, one or more optional targeting ligands 1004 can be present in the lipid layer 1003.

FIG. 1C provides an exemplary, non-limiting silica carrier having a silica shell that encapsulates a plasmid that targets a genomic sequence (e.g., by way of a CRISPR component that targets the genome of the host cell) or a phage that target a bacterial-derived genomic sequence (e.g., by way of a CRISPR component that targets either a bacterial genomic sequence present in the host's genomic sequence or present in a bacterium infecting the host cell). The carrier can be optimized to include surface ligands (e.g., first and second targeting ligands) that specifically target the desired cell. FIG. 1D shows an exemplary NanoCRISPR delivery platform (e.g., a protocell or a silica carrier) interacting with the host cell to deliver the biological package.

The protocell can be formed in any useful manner. As seen in the method 200 of FIG. 3A, a porous core 201 having a dimension d_{core} is first provided. Exemplary values for dimension d_{core} include, without limitation, greater than about 1 nm (e.g., greater than about 5 nm, 10 nm, 20 nm, 30 nm, 40 nm, 50 nm, 60 nm, 70 nm, 80 nm, 90 nm, 100 nm, 125 nm, 150 nm, 200 nm, 300 nm, 500 nm, 750 nm, 1 μm, 2 μm, 5 μm, 10 μm, 20 μm, or more).

Then, one or more cargos 202 are loaded 210 into the pores of core, in which the pore has a dimension dₚₒᵣₑ. Exemplary values for dimension dₚₒᵣₑ include, without limitation, greater than about 0.5 nm (e.g., around 0.5 nm to about 25 nm in diameter, often about 1 to around 20 nm in diameter).

A lipid layer 203 can be deposited 220 on an outer surface of the core (e.g., thereby forming a protocell 205). Furthermore, one or more optional targeting ligands 204 can be present in the lipid layer 203. The protocell can have any useful dimension, such as a diameter dₚ. Exemplary values for dimension dₚ include, without limitation, greater than about 10 nm (e.g., greater than about 20 nm, 30 nm, 40 nm, 50 nm, 60 nm, 70 nm, 80 nm, 90 nm, 100 nm, 125 nm, 150 nm, 200 nm, 300 nm, 500 nm, 750 nm, 1 μm, 2 μm, 5 μm, 10 μm, 20 μm, or more).

FIG. 3B provides an exemplary, non-limiting protocell containing cargo within pores or associating with cargo on an outer surface of the core for the protocell. For instance, the cargo can include a CRISPR component (e.g., Cas9/gRNA complex), vectors, metal-organic framework (if needed), and a phage that target a bacterial genomic sequence (e.g., by way of a CRISPR component that targets Bp). The carrier can be optimized to include surface ligands that specifically target the desired cell or pathogen.

As can be seen, additional components may be present in the delivery platform. In one instance, the delivery platform includes one or more components that facilitate CRISPR delivery to the target, such as modified CRISPR components with cell-penetrating peptides, co-delivery of CRISPR components with metal organic frameworks (MOFs) designed to permeabilize bacteria, and/or use of phage that encode CRISPR components. Additional details on the protocell, the silica carrier, the CRISPR/Cas system, biological package, and cargo are described herein.

In one instance, the particle includes a porous core (e.g., a silica core that is spherical and ranges in diameter from about 10 nm to about 250 nm (e.g., having a mean diameter of about 150 nm). In particular embodiments, silica core is monodisperse or polydisperse in size distribution.

In another instance, the particle includes an encapsulating shell (e.g., a silica shell configured to encapsulate the biological package). In further embodiments, the silica shell includes an outer surface and an inner surface, and the inner surface is disposed to be in proximity to the biological package. The shell can have any useful thickness (e.g., less than about 4 nm) and composed of any useful material (e.g., an inorganic material, a metal oxide, a silica, or an amorphous silica, each of which can be porous or non-porous).

A particle or a portion thereof (e.g., a protocell, a carrier, a core of the protocell, a shell of the carrier, etc.) may have a variety of shapes and cross-sectional geometries that may depend, in part, upon the process used to produce the particles. The particle can be a nanoparticle (e.g., having a diameter less than about 1 μm) or a microparticle (e.g., having a diameter greater than or equal to about 1 μm). In one embodiment, a particle may have a shape that is a sphere, a donut (torroidal), a rod, a tube, a flake, a fiber, a plate, a wire, a cube, or a whisker. A particle may include particles having two or more of the aforementioned shapes. In one embodiment, a cross-sectional geometry of the particle may be one or more of circular, ellipsoidal, triangular, rectangular, or polygonal. In one embodiment, a particle may consist essentially of non-spherical particles. For example, such particles may have the form of ellipsoids, which may have all three principal axes of differing lengths, or may be oblate or prelate ellipsoids of revolution. Non-spherical particles alternatively may be laminar in form, wherein laminar refers to particles in which the maximum dimension along one axis is substantially less than the maximum dimension along each of the other two axes. Non-spherical particles may also have the shape of frusta of pyramids or cones, or of elongated rods. In one embodiment, the particles may be irregular in shape. In one embodiment, a plurality of particles may consist essentially of spherical particles. Particles for use in the present invention may be PEGylated and/or aminated as otherwise described in PCT/US2014/56312 and PCT/US2014/56342, referenced above.

Characteristics of the Delivery Platform

A protocell generally includes a porous core and a supported lipid layer (e.g., a supported lipid bilayer (SLB)). In one instance, the core is a mesoporous silica nanoparticle (MSNP). In another instance, the core optionally includes a cell-permeabilizing metal organic framework. One or more cargoes can be disposed within a plurality of pores of the core. Optionally, cargo(s) can be linked to the SLB (e.g., by a linker, such as any described herein).

A silica carrier generally includes a biological package encapsulated in a silica shell and can optionally include a supported lipid layer (e.g., a supported lipid bilayer or supported lipid multilayer having more than three lipid layers). One or more cargoes can be disposed within the silica shell and/or with the biological package within the shell.

The particle size distribution (e.g., size of the core for the protocell or a size of the silica carrier), according to the present invention, depends on the application, but is principally monodisperse (e.g., a uniform sized population varying no more than about 5-20% in diameter, as otherwise described herein). In certain embodiments, particles can range, e.g., from around 1 nm to around 500 nm in size, including all integers and ranges there between. The size is measured as the longest axis of the particle. In various embodiments, the particles are from around 5 nm to around 500 nm and from around 10 nm to around 100 nm in size.

The particles can have a porous structure (e.g., as a core or as a shell). The pores can be from around 0.5 nm to about 25 nm in diameter, often about 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

In certain embodiments, preferred MSNPs, protocells, or carriers according to the present invention: are monodisperse and range in size from about 25 nm to about 300 nm; exhibit stability (colloidal stability); have single cell binding specification to the substantial exclusion of non-targeted cells; are anionic, neutral or cationic for specific targeting (preferably cationic); are optionally modified with agents such as PEI, NMe³⁺, dye, crosslinker, ligands (ligands provide neutral charge); and optionally, are used in combination with a cargo to be delivered to the target.

In certain alternative embodiments, the MSNPs, protocells, or carriers are monodisperse and range in size from about 25 nm to about 300 nm. The sizes used preferably include 50 nm (+/−10 nm) and 150 nm (+/−15 nm), within a narrow monodisperse range, but may be more narrow in range.

In certain alternative embodiments, the present invention are directed to MSNPs and preferably, protocells, or carriers of a particular size (diameter) ranging from about 0.5 to about 30 nm, about 1 nm to about 30 nm, often about 5 nm to about 25 nm (preferably, less than about 25 nm), often about 10 to about 20 nm, for administration in any useful route. In some embodiments, these MSNPs, protocells, and/or carriers are often monodisperse and provide colloidally stable compositions. These compositions can be used to target host cells because of enhanced biodistribution/bioavailability of these compositions, and optionally, specific cells, with a wide variety of therapeutic and/or diagnostic agents that exhibit varying release rates at the site of activity.

The particles (e.g., having a core or a shell) can be produced in any useful manner. In one instance, particles with 7.9 nm pores (e.g., in the core or in the shell) can be prepared with templating by Pluronic® F127. In another instance, the particles include 18-25 nm pores (see, e.g., Gao F et al., J. Phys. Chem. B. 2009; 113:1796-804). In yet another instance, the pores can be templated with cross-linked micelles, thereby providing pores with precise diameters ranging from 10 nm to 20 nm. Various sizes of cross-linked micelles will be prepared by mixing various concentrations of Pluronic® F127 with polypropylene oxide, 25% tetrahydrofuran, and benzoyl peroxide; the resulting micelle solution will then be aged for 24 hours at 60° C., vacuum dried, and added to the silica precursor solution. Each batch of particles can be characterized in any useful manner, such as by assessment of size and surface charge using dynamic light scattering (DLS) (NIST-NCL PCC-1 and PCC-2) and electron microscopy (NIST-NCL PCC-7 and PCC-15) and verification of low endotoxin contamination per health industry product standards (NCL STE-1.1). In addition, ten percent of particle (e.g., NanoCRISPR) batches will be randomly tested for solvent and surfactant contamination using mass spectrometry.

To enable burst release of CRISPR components (e.g., guiding component(s) and nuclease component(s), including the nuclease or a nucleic acid sequence that encodes the nuclease) in the cytosol of host cells, pore-templating surfactants and cross-linked micelles can be extracted (e.g., using acidified ethanol to minimize the degree of silica condensation in the particle framework). Furthermore, if the cargo has an isoelectric points or pKa values <7, then naturally negatively-charged particles can be modified with amine-containing silanes (e.g., (3-aminopropyl) triethoxysilane, or APTES) in order to maximize electrostatic interactions between pore walls and cargo molecules.

The core of a protocell can be loaded in any useful manner. For instance, loading with CRISPR components, alone and in combination with small molecule antimicrobials, can be accomplished by soaking the MSNP with the cargo (see, e.g., Ashley C E et al., ACS Nano 2012; 6:2174-88; Ashley C E et al., Nat. Mater. 2011; 10: 389-97; and Epler K et al., Adv. Healthc. Mater. 2012 1:348-53). Loading capacities for Cas9/guiding component complexes and other agents (e.g., small molecule antimicrobials and/or antivirals) can be determined in any useful manner (e.g., using spectrophotometer and absorbance or fluorescence-based HPLC methods). Release rates can be confirmed upon encapsulation of cargo-loaded MSNPs in an SLB (e.g., a DOPC SLB) and dispersion in simulated body and/or endolysosomal fluids.

Pore size of the core can be modified, as needed, to accommodate the CRISPR components, as well as any other cargo. We have previously shown that MSNPs with 18-25 nm pores can be loaded with high concentrations of minicircle DNA vectors up to 2000-bp in size, as well as histone-packaged plasmids up to 6000-bp in size via our simple soaking procedure (see e.g., Ashley C E et al., ACS Nano 2012; 6:2174-88; Ashley C E et al., Nat. Mater. 2011; 10: 389-97; and Epler K et al., Adv. Healthc. Mater. 2012 1:348-53). To minimize possible anti-histone antibody responses in vivo (e.g., arising from pre-packaged plasmids within the core), the cargo can be entrapped within the MSNPS as they are being formed in EISA reactors. Such cargo can include any herein, such as linear and circular DNA vectors of various sizes.

Alternatively, CRISPR components can be encapsulated within a silica shell, as in a silica carrier. In this configuration, large CRISPR components (e.g., having a dimension greater than about 20 nm or having more than about 6.000-bp) can be obtained, and the biodegradable silica shell can be built around the CRISPR component(s). In this manner, self-assembly processes provide no limit as to the size of the biological package that can be encapsulated in the silica shell. Of course, carrier size can affect biodistribution and cellular uptake, which can be controlled in the manner described herein.

Cargo can be introduced to the core in any useful manner. For instance, the cargo can be introduced (e.g., by soaking) after the MSNP is synthesized. Alternatively, cargo can be introduced during MSNP or silica shell synthesis. In yet another instance, cargo is complexed with the biological package prior to encapsulation with a silica shell. In another instance, the cargo is introduced (e.g., by soaking) after the silica shell of the carrier is synthesized.

In one instance, cargo can be introduced at various concentrations into the precursor solution, which will then aerosolize and pass through the reactor at high flow rates to minimize exposure of the cargo to high temperatures (e.g., <1 second in the 400° C. heating zone). Within each aerosolized droplet, silica will self-assemble around the cargo (e.g., DNA molecules), resulting in nanoparticles that entrap the cargo. For a cargo being DNA, preliminary experiments indicate we can entrap ˜0.3 mg of a 3300 bp DNA vector per mg of MSNPs and that, upon dissolution of the silica framework, the DNA vector, which encodes expression of a fluorescent reporter protein (ZsGreen), is able to transfect Vero cells. These data indicate that the process does not damage the vector. Similar methodologies can be employed to entrap any useful agent, such as a cargo (e.g., phage) or a MOF.

Co-loading of cargos can also be implemented in any useful manner. For instance, to enable co-loading of DNA- and phage-based countermeasures with small molecule antimicrobials, cetyltrimethylammonium bromide (CTAB) can be employed in the precursor solution to template 2.5 nm pores in resulting MSNPs. Then, CTAB can be extracted using acidified ethanol to promote burst release rates.

Biological Packages and Cargos

The delivery platform can include any useful biological package or cargo, including CRISPR components, as well as other cargos (e.g., either associated with the nanoparticle core or the supported lipid bilayer). Biological packages or cargos can include a variety of molecules, including peptides, proteins, aptamers, and antibodies. For instance, combinatorial screens can be performed to identify synergistic effects between CRISPR-based countermeasures or CRISPR components in combination with other agents (e.g., small molecule drugs, such as antimicrobials and/or antivirals, an agrochemical, a carbohydrate, a dye, a marker, a nutrient, a penetrant, and/or a surfactant, or any other agent described herein).

Exemplary biological packages and/or cargos include an acidic, basic, and hydrophobic drug (e.g., antiviral agents, antibiotic agents, etc.); a protein (e.g., antibodies, carbohydrates, etc.); a nucleic acid (e.g., DNA, RNA, small interfering RNA (siRNA), minicircle DNA (mcDNA) vectors, e.g., that encode small hairpin RNA (shRNA), complementary DNA (cDNA), naked DNA, and plasmid DNA, as well as chimeras, single-stranded forms, duplex forms, and multiplex forms thereof); a diagnostic/contrast agent, like quantum dots, iron oxide nanoparticles, gadolinium, and indium-111; a small molecule; a drug, a pro-drug, a vitamin, an antibody, a protein, a hormone, a growth factor, a cytokine, a steroid, an anticancer agent, a fungicide, an antimicrobial, an antibiotic, etc.; a morphogen; a toxin, e.g., a bacterial protein toxin; a peptide, e.g., an antimicrobial peptide; an antigen; an antibody; a detection agent (e.g., a particle, such as a conductive particle, a microparticle, a nanoparticle, a quantum dot, a latex bead, a colloidal particle, a magnetic particle, a fluorescent particle, etc.; or a dye, such as a fluorescent dye, a luminescent dye, a chemiluminescent dye, a colorimetric dye, a radioactive agent, an electroactive detection agent, etc.); a label (e.g., a quantum dot, a nanoparticle, a microparticle, a barcode, a fluorescent label, a colorimetric label, a radio label (e.g., an RF label or barcode), avidin, biotin, a tag, a dye, a marker, an electroactive label, an electrocatalytic label, and/or an enzyme that can optionally include one or more linking agents and/or one or more dyes); a capture agent (e.g., such as a protein that binds to or detects one or more markers (e.g., an antibody or an enzyme), a globulin protein (e.g., bovine serum albumin), a nanoparticle, a microparticle, a sandwich assay reagent, a catalyst (e.g., that reacts with one or more markers), and/or an enzyme (e.g., that reacts with one or more markers, such as any described herein)); as well as combinations thereof.

In some instances, the biological package includes biological package a nucleic acid and/or a polypeptide. The nucleic acid can be provided in any useful form, such as RNA, DNA, DNA/RNA hybrids, phage, plasmid, linear forms thereof, concatenated forms thereof, circularized forms thereof, modified forms thereof, single stranded forms thereof, and double stranded forms thereof.

The biological package or cargo can optionally include a plasmid. The plasmid can encode any useful CRISPR component (e.g., a guiding component or a nuclease). In addition, the plasmid can express any useful polypeptide and/or nucleic acid sequence, including a nuclear localization sequence, a cell penetrating peptide, a targeting peptide, a polypeptide toxin, a small hairpin RNA (shRNA), a small interfering RNA (siRNA), a reporter (e.g., a reporter protein), etc. Additional reporters include polypeptide reporters which may be expressed by plasmids (such as histone-packaged supercoiled DNA plasmids) and include polypeptide reporters such as fluorescent green protein and fluorescent red protein. Reporters pursuant to the present invention are utilized principally in diagnostic applications including diagnosing the existence or progression of cancer (cancer tissue) in a patient and or the progress of therapy in a patient or subject.

The plasmid can be of any useful form (e.g., supercoiled and/or packaged plasmid). For instance, the plasmid can be a histone-packaged supercoiled plasmid including a mixture of histone proteins.

Any useful cargo, including combinations thereof, can be included within the delivery platform. Exemplary cargos include a nucleic acid, a polypeptide, a small molecule, an agrochemical, a carbohydrate, a dye, a marker, a nutrient, a penetrant, and/or a surfactant. Exemplary nucleic acids include DNA (e.g., double stranded or linear DNA, complementary DNA (cDNA), minicircle DNA, naked DNA, or alternative plasmid DNA), RNA (e.g., mRNA, siRNA, shRNA, or micro RNA), as well as modified forms thereof.

Exemplary agrochemicals include fungicides, insecticides, pesticides, biopesticides (e.g., plant-incorporated-protectants, microbial pesticides, or biochemical pesticides), nematicides, fertilizers, growth agents, and herbicides. Exemplary nutrients include minerals (e.g., phosphate, phosphite, ammonia, ammonium, carbonic acid, carbonate salts, potassium, etc., including salts thereof) or micronutrients (e.g., boron, chlorine, copper, iron, manganese, molybdenum, and zinc).

Exemplary penetrants include polyalkoxytriglycerides, including those having ethoxylated, propoxylated, and/or butoxylated side chains, in which the length of the unmodified side chains can vary from 9 to 24, preferably from 12 to 22, very preferably from 14 to 20, carbon atoms independently of the other side chains in the same molecule. These aliphatic side chains can be straight-chain or branched. Non-limiting ethoxylated triglycerides include ethoxylated rapeseed oil, maize oil, palm kernel oil or almond oil. Corresponding polyalkoxytriglycerides are known or can be prepared by known methods (commercially available, for example, under the names Crovol® A 70 UK. Crovol® CR 70 G, Crovol® M 70 and Crovol PK 70 from Croda).

Exemplary surfactants include nonionic surfactants and anionic surfactants, including polyethylene oxide/polypropylene oxide block copolymers, polyethylene glycol ethers of straight-chain alcohols, reaction products of fatty acids with ethylene oxide and/or propylene oxide, furthermore polyvinyl alcohol, polyvinylpyrrolidone, mixed polymers of polyvinyl alcohol and polyvinylpyrrolidone, mixed polymers of polyvinyl acetate and polyvinylpyrrolidone copolymers of (meth)acrylic acid and (meth)acrylic esters, alkyl ethoxylates, alkylaryl ethoxylates, alkylsulphonic acids (e.g., including alkali metal and alkaline earth metal salts thereof), alkylarylsulphonic acids (e.g., including alkali metal and alkaline earth metal salts thereof), polystyrenesulphonic acids (e.g., including salts thereof), salts of polyvinylsulphonic acids (e.g., including salts thereof), naphthalenesulphonic acid (e.g., including salts thereof and/or formaldehyde condensates thereof, such as salts of condensates of naphthalenesulphonic acid), phenolsulphonic acid (e.g., including salts and/or formaldehyde condensates thereof), and lignosulphonic acid (e.g., including salts thereof), as well as combinations thereof.

MSNPs pursuant to the present invention may be used to deliver cargo to a targeted host cell, including, for example, cargo component selected from the group consisting of at least one polynucleotide, such as double stranded linear DNA, minicircle DNA, naked DNA or plasmid DNA (especially CRISPR ds plasmid DNA, RNA, as well as chimeras, fusions, or modified forms thereof), messenger RNA, small interfering RNA, small hairpin RNA, microRNA, a polypeptide (e.g., a recruitment domain or fragments thereof), a protein (e.g., an enzyme, an initiation factor, or fragments thereof), a drug (in particular, an anticancer drug such as a chemotherapeutic agent), an imaging agent, a detection agent (e.g., a dye, such as an electroactive detection agent, a fluorescent dye, a luminescent dye, a chemiluminescent dye, a colorimetric dye, a radioactive agent, etc.), a label (e.g., a fluorescent label, a colorimetric label, a quantum dot, a nanoparticle, a microparticle, an electroactive label, an electrocatalytic label, a barcode, a radio label (e.g., an RF label or barcode), avidin, biotin, a tag, a dye, a marker, an enzyme or protein that can optionally include one or more linking agents and/or one or more dyes), or a mixture thereof. The MSNPs pursuant to the present invention are effective for accommodating cargo which are long and thin (e.g., naked) in three-dimensional structure, such as polynucleotides (e.g., various DNA and RNA) and polypeptides.

Targeting Ligands

The biological package and/or cargo can include one or more cell targeting species, cell penetrating peptides, fusogenic peptides, and/or targeting peptides. Such species can be included within the biological package or cargo, configured to be expressed by a plasmid of the biological package or cargo, and/or located within a lipid layer supported on a surface of the particle.

In some instances, the targeting ligand can be a cell penetration peptide, a fusogenic peptide, or an endosomolytic peptide, which are peptides that aid a MSNP, a protocell, or a carrier in translocating across a lipid bilayer, such as a cellular membrane or endosome lipid bilayer of the host cell. In one embodiment, the targeting ligand is optionally crosslinked onto a lipid layer surface of the protocells or carriers according to the present invention.

Endosomolytic peptides are a sub-species of fusogenic peptides as described herein. In both the multilamellar and single layer protocell or carrier embodiments, the non-endosomolytic fusogenic peptides (e.g., electrostatic cell penetrating peptide such as R8 octaarginine) are incorporated onto the protocells or carriers at the surface of the protocell or carrier in order to facilitate the introduction of protocells or carriers into targeted cells (APCs) to effect an intended result (to instill an immunogenic and/or therapeutic response as described herein). The endosomolytic peptides (often referred to in the art as a subset of fusogenic peptides) may be incorporated in the surface lipid bilayer of the protocell or carrier or in a lipid sublayer of the multilamellar protocell or carrier in order to facilitate or assist in the escape of the protocell or carrier from endosomal bodies.

Representative and preferred electrostatic cell penetration (fusogenic) peptides for use in protocells or carriers according to the present invention include an 8 mer polyarginine (NH₂-RRRRRRRR-COOH, SEQ ID NO: 1), among others known in the art, which are included in protocells according to the present invention in order to enhance the penetration of the protocell or carrier into cells.

Representative endosomolytic fusogenic peptides (“endosomolytic peptides”) include H5WYG peptide (NH₂-GLFHAIAHFIHGGWHGLIHGWYGGC-COOH, SEQ ID NO:2), RALA peptide (NH₂-WEARLARALARALARHLARALARALRAGEA-COOH, SEQ ID NO:3), KALA peptide (NH₂-WEAKLAKALAKALAKHLAKALAKALKAGEA-COOH), SEQ ID NO:4), GALA (NH₂-WEAALAEALAEALAEHLAEALAEALEALAA-COOH, SEQ ID NO:5) and INF7 (NH₂-GLFEAIEGFIENGWEGMIDGWYG-COOH, SEQ ID NO:6), or fragments thereof, among others. In one instance, the targeting ligand includes an amino acid sequence having at least 80% sequence identity (e.g., at least 85%, 90%, 95%, or 99% sequence identity) to any one of SEQ ID NOs: 1-6, or a fragment thereof.

Other exemplary targeting ligands include poly-L-arginine, including (R)ₙ, where 6<n<12, such as an R12 peptide (e.g., RRRRRRRRRRRR (SEQ ID NO:210)) or an R9 peptide (e.g., RRRRRRRRR (SEQ ID NO:211)); a poly-histidine-lysine, such as a (KH)₉ (e.g., KHKHKHKHKHKHKHKHKH (SEQ ID NO:212)); a Tat protein or derivatives and fragments thereof, such as RKKRRQRRR (SEQ ID NO:213), GRKKRRQRRRPQ (SEQ ID NO:214), GRKKRRQRRR (SEQ ID NO:215), GRKKRRQRRRPPQ (SEQ ID NO:216), YGRKKRRQRRR (SEQ ID NO:217), and RKKRRQRRRRKKRRQRRR (SEQ ID NO:218); a Cady protein or derivatives and fragments thereof, such as Ac-GLWRALWRLLRSLWRLLWRA-cysteamide (SEQ ID NO:219); a penetratin protein or derivatives and fragments thereof, such as RQIKIWFQNRRMKWKKGG (SEQ ID NO:220), RQIRIWFQNRRMRWRR (SEQ ID NO:221), and RQIKIWFQNRRMKWKK (SEQ ID NO:222); an antitrypsin protein or derivatives and fragments thereof, such as CSIPPEVKFNKPFVYLI (SEQ ID NO:223); a temporin protein or derivatives and fragments thereof, such as FVQWFSKFLGRIL-NH₂ (SEQ ID NO:224); a MAP protein or derivatives and fragments thereof, such as KLALKLALKALKAALKLA (SEQ ID NO:225); a RW protein or derivatives and fragments thereof, such as RRWWRRWRR (SEQ ID NO:226); a pVEC protein or derivatives and fragments thereof, such as LLIILRRRIRKQAHAHSK (SEQ ID NO:227); a transportan protein or derivatives and fragments thereof, such as GWTLNSAGYLLGKINLKALAALAKKIL (SEQ ID NO:228); a MPG protein or derivatives and fragments thereof, such as GALFLGFLGAAGSTMGAWSQPKKKRKV (SEQ ID NO:229); a Pep protein or derivatives and fragments thereof, such as KETWWETWWTEWSQPKKKRKV (SEQ ID NO:230), Ac-KETWWETWWTEWSQPKKKRKV-cysteamine (SEQ ID NO:231), and WKLFKKILKVL-amide (SEQ ID NO:232); a Bp100 protein or derivatives and fragments thereof, such as KKLFKKILKYL (SEQ ID NO:233) and KKLFKKILKYL-amide (SEQ ID NO:234); a maurocalcine protein or derivatives and fragments thereof, such as GDC(acm)LPHLKLC (SEQ ID NO:235); a calcitonin protein or derivatives and fragments thereof, such as LGTYTQDFNKFHTFPQTAIGVGAP (SEQ ID NO:236); a neurturin protein or derivatives and fragments thereof, such as GAAEAAARVYDLGLRRLRQRRRLRRERVRA (SEQ ID NO:237); and a human P1 protein or derivatives and fragments thereof, such as MGLGLHLLVLAAALQGAWSQPKKKRKV (SEQ ID NO:238).

Yet other exemplary targeting ligands include a targeting peptide (e.g., plastid transit peptides or signal peptides, such as those described in U.S. Pat. Nos. 5,977,437, 8,084,666 and 8,791,325, and U.S. Pat. Pub. Nos. 2009/0328249 and 2010/0279390, each of which is incorporated herein by reference in its entirety). In some instances, the targeting ligand is a chloroplast transit peptide or a mitochondrial transit peptide, such as MGGCVSTPKSCVGAKLR (SEQ ID NO:240), MQTLTASSSVSSIQRHRPHPAGRRSSSVTFS (SEQ ID NO:241), MKNPPSSFASGFGIR (SEQ ID NO:242), MAALIPAIASLPRAQVEKPHPMPVSTRPGLVS (SEQ ID NO:243), MSSPPPLFTSCLPASSPSIRRDSTSGSVTSPLR (SEQ ID NO:244), MFSYLPRYPLRAASARALVRATRPSYRYALLRYQ (SEQ ID NO:245), X₁X₂X₃X₄X₅X₆VX₈AX₁₀X₁₁X₁₂P (SEQ ID NO:246, where X₁ is R, S, G, or A; each of X₂ and X₁₁ is, independently, R or A; X₃ is R, S, V, or A; each of X₄ and X₁₀ is, independently. A, S, R, or F; X₅ is V or L; each of X₆ and X₈ is, independently, V or R; and X₁₂ is any amino acid, e.g., E, L, V, Q, A, R, and S), X₁RX₃X₄X₅VVRAX₁₀AX₁₂P (SEQ ID NO:247, where each of X₁ and X₃ is, independently, R or S; each of X₄ and X₁₀ is, independently, A or S; X₅ is V or L; and X₁₂ is any amino acid, e.g., E, L, Q, A, R, and S), X₁X₂RX₄X₅AX₇AAX₁₀X₁₁ (SEQ ID NO:248, where X₁ is G, A, or F; X₂ is V, L, Q, or S; X₄ is A, G, or T; X₅ is F, S, or Y; X₇ is T or A; X₁₀ is A or S; and X₁₁ is any amino acid, e.g., D, A, G, S, or F), and X₁VRAFAX₇AAAX₁₁ (SEQ ID NO:249, where X₁ is G, A, or F; X₇ is T or A; and X₁₁ is any amino acid, e.g., D, A, G, S, or F).

In one instance, the targeting ligand includes an amino acid sequence having at least 80% sequence identity (e.g., at least 85%, 90%, 95%, or 99% sequence identity) to any one of SEQ ID NOs:210-238, and 240-249, or a fragment thereof (e.g., having a length of about 4, 5, 6, 7, 8, 9, 10, 11, 12, 13, 14, 15, 16, 17, 18, 19, 20, 22, 24, 26, 28, 30, 32, 34, 36, 38, 40, or more amino acids).

Proteins gain entry into the nucleus through the nuclear envelope. The nuclear envelope consists of concentric membranes, the outer and the inner membrane. These are the gateways to the nucleus. The envelope consists of pores or large nuclear complexes. A protein translated with a NLS will bind strongly to importin (aka karyopherin), and together, the complex will move through the nuclear pore. Any number of nuclear localization sequences may be used to introduce histone-packaged plasmid DNA into the nucleus of a cell. Preferred nuclear localization sequences include NH₂-GNQSSNFGPMKGGNFGGRSSGPYGGGGQYFAKPRNQGGYGGC-COOH (SEQ ID NO:9), RRMKWKK (SEQ ID NO:10), PKKKRKV (SEQ ID NO: 11), and KR[PAATKKAGQA]KKKK (SEQ ID NO: 12), the NLS of nucleoplasmin, a prototypical bipartite signal comprising two clusters of basic amino acids, separated by a spacer of about 10 amino acids. Numerous other nuclear localization sequences are well known in the art. See, for example, LaCasse E C et al., “Nuclear localization signals overlap DNA- or RNA-binding domains in nucleic acid-binding proteins,” Nucl. Acids Res. 1995; 23:1647-56; Weis, K, “Importins and exportins: how to get in and out of the nucleus,” [published erratum appears in Trends Biochemn. Sci. 1998 July; 23(7):235]Trends Biochem. Sci. 1998; 23:185-9; Cokol M et al., EMBO Rep. 2000 Nov. 15; 1(5): 411-5; and Murat Cokol, Raj Nair & Burkhard Rost, “Finding nuclear localization signals”, at the website ubic.bioc.columbia.edu/papers/2000 nls/paper.html#tab2, each of which is incorporated herein by reference in its entirety.

The charge is controlled based on what is to be accomplished (via PEI, NMe^{3+,} dye, crosslinker, ligands, etc.), but for targeting the charge is preferably cationic. Charge also changes throughout the process of formation. Initially the targeted particles are cationic and are often delivered as cationically charged nanoparticles, however post modification with ligands they are closer to neutral. The ligands which find use in the present invention include peptides, affibodies, and antibodies, among others. These ligands are site specific and are useful for targeting specific cells which express peptides to which the ligand may bind selectively to targeted cells.

The composition of the lipid layer can include one or more components that facilitate ligand orientation, maximize cellular interaction, provide lipid stability, and/or confer enhanced cellular entry. In one instance, to ensure that targeting ligands are properly oriented on the NanoCRISPR surface, the SLB composition can include DOPC with 30 wt % cholesterol and 5-10 wt % of 1,2-dioleoyl-sn-glycero-3-phosphoethanolamine (DOPE), to which we will conjugate peptides or scFvs with C-terminal cysteine residues using a commercially-available, heterobifunctional amine-to-sulfhydryl crosslinker (SM(PEG)₂₄). The minimum density of targeting ligands necessary can be determined to maximize specific interactions between NanoCRISPRs and model host cells using flow cytometry or surface plasmon resonance to quantify thermodynamic (e.g., dissociation constants) and kinetic (on and off rate constants) binding constants. In another instance, the lipid bilayer includes a phase-separated lipid bilayer.

Lipid Layers

The delivery platform can optionally include a supported lipid layer. Numerous lipids which are used in liposome delivery systems may be used to form the lipid bi- or multilayer on particles (e.g., nanoparticles) to provide MSNPS, protocells, and/or carriers according to the present invention.

The lipid layer can include any useful lipid or combination of lipids, such as one or more lipids selected from the group of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine](DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dilauroyl-sn-glycero-3-phosphocholine (DLPC), 1,2-dimyristoyl-sn-glycero-3-phosphocholine (DMPC), 1-palmitoyl-2-oleoyl-sn-glycero-3-phosphocholine (POPC), 1-stearoyl-2-oleoyl-sn-glycero-3-phosphocholine (SOPC), 1,2-dioleoyl-sn-glycero-3-phosphoethanolamine-N-[methoxy(polyethylene glycol)-2000](18:1 PEG-2000 PE), 1,2-dipalmitoyl-sn-glycero-3-phosphoethanolamine-N-[methoxy(polyethylene glycol)-2000](16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol, and mixtures thereof.

Cholesterol, not technically a lipid, but presented as a lipid for purposes of an embodiment of the present invention given the fact that cholesterol may be an important component of the lipid bilayer of protocells or carriers according to an embodiment of the invention. Often cholesterol is incorporated into lipid bilayers of protocells or carriers in order to enhance structural integrity of the bilayer. These lipids are all readily available commercially from Avanti Polar Lipids, Inc. (Alabaster, Ala., USA). DOPE and DPPE are particularly useful for conjugating (through an appropriate crosslinker) PEG, peptides, polypeptides, including immunogenic peptides, proteins and antibodies, RNA and DNA through the amine group on the lipid.

MSNPs, protocells, and/or carriers of the invention can be PEGylated with a variety of polyethylene glycol-containing compositions as described herein. PEG molecules can have a variety of lengths and molecular weights and include, but are not limited to, PEG 200, PEG 1000, PEG 1500, PEG 4600, PEG 10,000, PEG-peptide conjugates or combinations thereof.

In one instance, the lipid layer includes DOPC and DOPE. In another instance, the lipid layer includes a zwitterionic lipid (e.g., DOPC, DPPC, DOPE, DPPE, DLPC, DMPC, POPC, or SOPC) with an optional PEG (e.g., PEG, PEG-2000 PE, PEG conjugated to DOPE, PEG conjugated to DPPE, etc.).

In yet another instance, the lipid layer includes DOTAP, DOPG, DOPC, or mixtures thereof. In another instance, the lipid layer includes PEG. In yet another instance, the lipid layer includes cholesterol. In another instance, the lipid layer includes DOPG and DOPC. In one instance, the lipid layer includes DOPC in combination with about 5 wt % DOPE, about 30 wt % cholesterol, and about 10 wt % PEG-2000 PE (18:1). In another instance, the lipid layer includes about 5% by weight DOPE, about 5% by weight PEG, about 30% by weight cholesterol, about 60% by weight DOPC and/or DPPC.

The lipid bi- or multilayer supported on the porous particle according to one embodiment of the present invention has a lower melting transition temperature, i.e., is more fluid than a lipid bi- or multilayer supported on a non-porous support or the lipid bi- or multilayer in a liposome. This is sometimes important in achieving high affinity binding of immunogenic peptides or targeting ligands at low peptide densities, as it is the bilayer fluidity that allows lateral diffusion and recruitment of peptides by target cell surface receptors. One embodiment provides for peptides to cluster, which facilitates binding to a complementary target.

In the present invention, the lipid bi- or multilayer may vary significantly in composition. Ordinarily, any lipid or polymer which may be used in liposomes may also be used in MSNPs, protocells, or carriers according to the present invention. Preferred lipids are as otherwise described herein.

In embodiments according to the invention, the lipid bi- or multilayer of the protocells or the carriers can provide biocompatibility and can be modified to possess targeting species including, for example, antigens, targeting peptides, fusogenic peptides, antibodies, aptamers, and PEG (polyethylene glycol) to allow, for example, further stability of the protocells or carriers and/or a targeted delivery into a cell to maximize an immunogenic response. PEG, when included in lipid bilayers, can vary widely in molecular weight (although PEG ranging from about 10 to about 100 units of ethylene glycol, about 15 to about 50 units, about 15 to about 20 units, about 15 to about 25 units, about 16 to about 18 units, etc, may be used) and the PEG component which is generally conjugated to phospholipid through an amine group comprises about 1% to about 20%, preferably about 5% to about 15%, about 10% by weight of the lipids which are included in the lipid bi- or multilayer. The PEG component is generally conjugated to an amine-containing lipid such as DOPE or DPPE or other lipid, but in alternative embodiments may also be incorporated into the MSNPs, through inclusion of a PEG containing silane.

CRISPR/Cas Components

The present invention relates to a delivery platform including one or more CRISPR components (e.g., associated with the core, within the shell, and/or the supported lipid bilayer). FIG. 6A-6C shows a CRISPR component and its non-limiting use with a delivery platform described herein. The CRISPR/Cas system evolved naturally within prokaryotes to confer resistance to exogenous genetic sequences (FIG. 6A-6B). As can be seen (FIG. 6A), the CRISPR/Cas system can include a CRISPR array that is a noncoding RNA transcript that is further cleaved into CRISPR RNA (crRNA), a trans-acting CRISPR RNA (tracrRNA), and various CRISPR-associated (Cas) proteins.

This CRISPR/Cas system can be adapted to control genetic expression in targeted manner, such as, e.g., by employing synthetic, non-naturally occurring constructs that use crRNA nucleic acid sequences, tracrRNA nucleic acid sequences, and/or Cas polypeptide sequences, as well as modified forms thereof.

One CRISPR component includes a guiding component. In general, the guiding component includes a nucleic acid sequence (e.g., a single polynucleotide) that includes at least two portions: (1) a targeting portion, which is a nucleic acid sequence that imparts specific targeting to the target genomic locus (e.g., a guide RNA or gRNA); and an interacting portion, which is another nucleic acid sequence that binds to a nuclease (e.g., a Cas endonuclease). In some instances, the interacting portion includes two particular sequences that bind the nuclease, e.g., (2) a short crRNA sequence attached to the guide nucleic acid sequence; and (3) a tracrRNA sequence attached to the crRNA sequence. Exemplary targeting CRISPR components include a minicircle DNA vector optimized for in vivo expression.

Another CRISPR component includes a nuclease (e.g., that binds the targeting nucleic acid sequence). The nuclease CRISPR component can either be an enzyme, or a nucleic acid sequence that encodes for that enzyme. Exogenous endonuclease (e.g., Cas9) can be encoded by a cargo stored within the protocell and/or the silica carrier. Any useful nuclease can be employed, such as Cas9 (e.g., SEQ ID NO: 110), as well as nickase forms and deactivated forms (e.g., SEQ ID NO: 111) thereof (e.g., including one or more mutations, such as D10A, H840A, N854A, and N863A in SEQ ID NO: 110 or in an amino acid sequence sufficiently aligned with SEQ ID NO: 110), including nucleic acid sequences that encode for such nuclease. Pathogen-directed and host-directed CRISPR components (e.g., guiding components and/or nuclease), as well as minicircle DNA vectors that encode Cas and guiding components can be developed. The nuclease can be configured to bind the target sequence and/or cleave the target sequence.

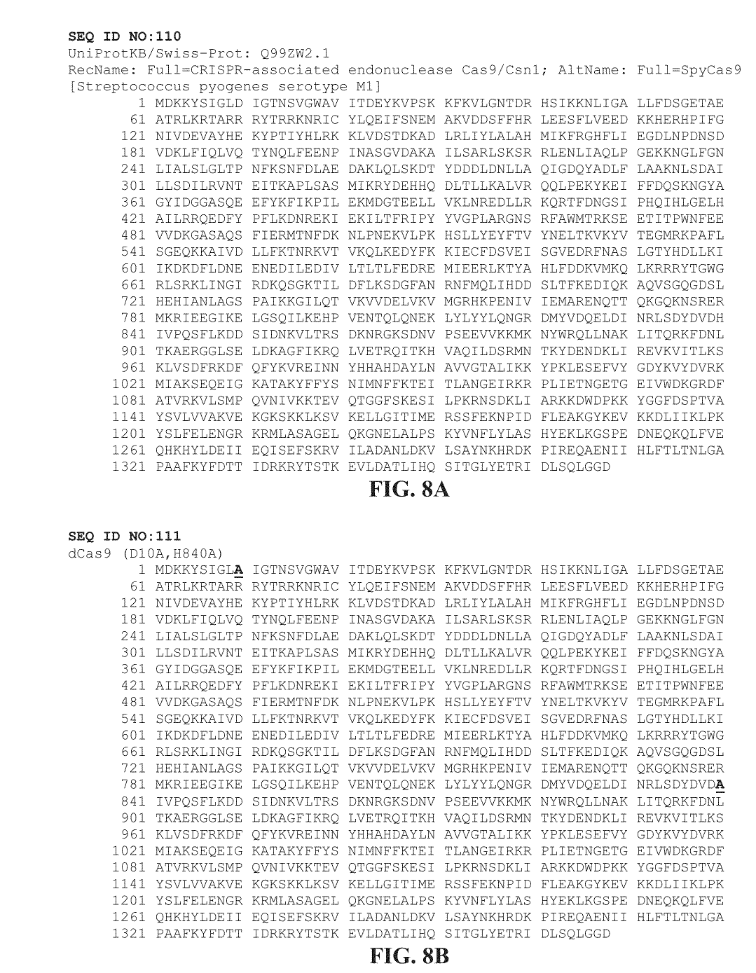

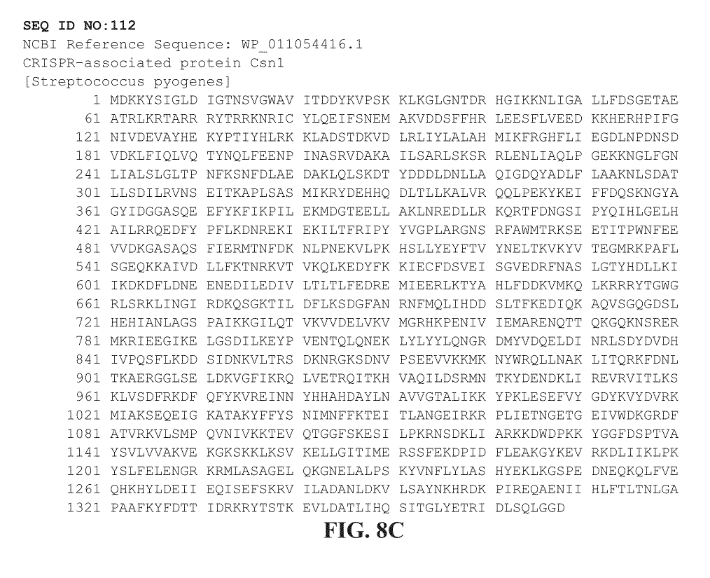

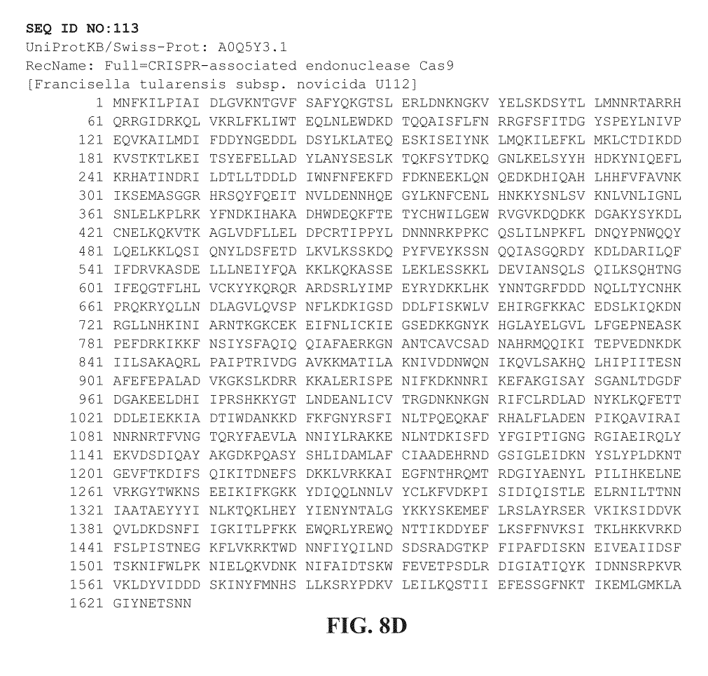

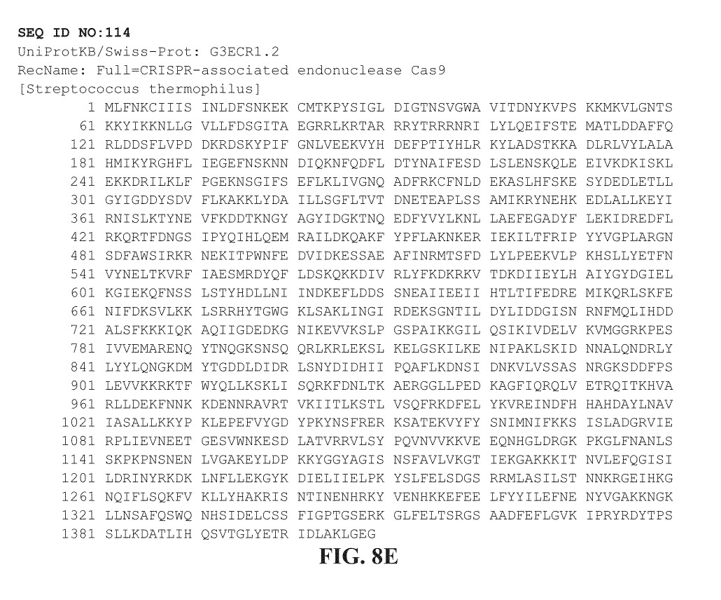

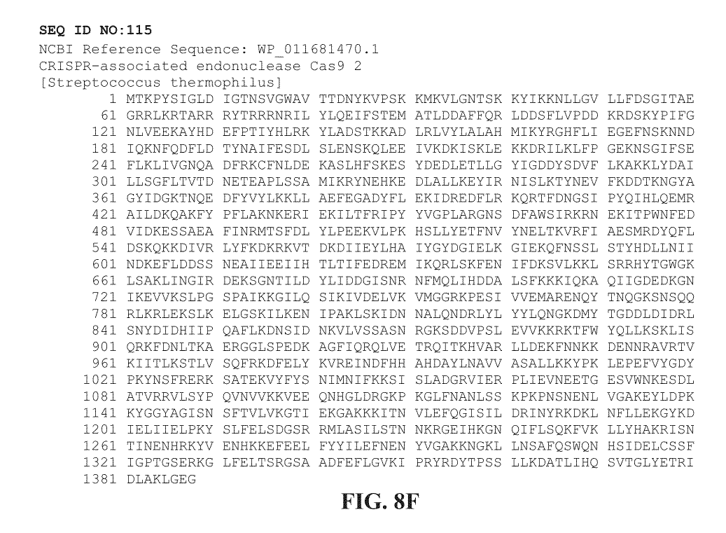

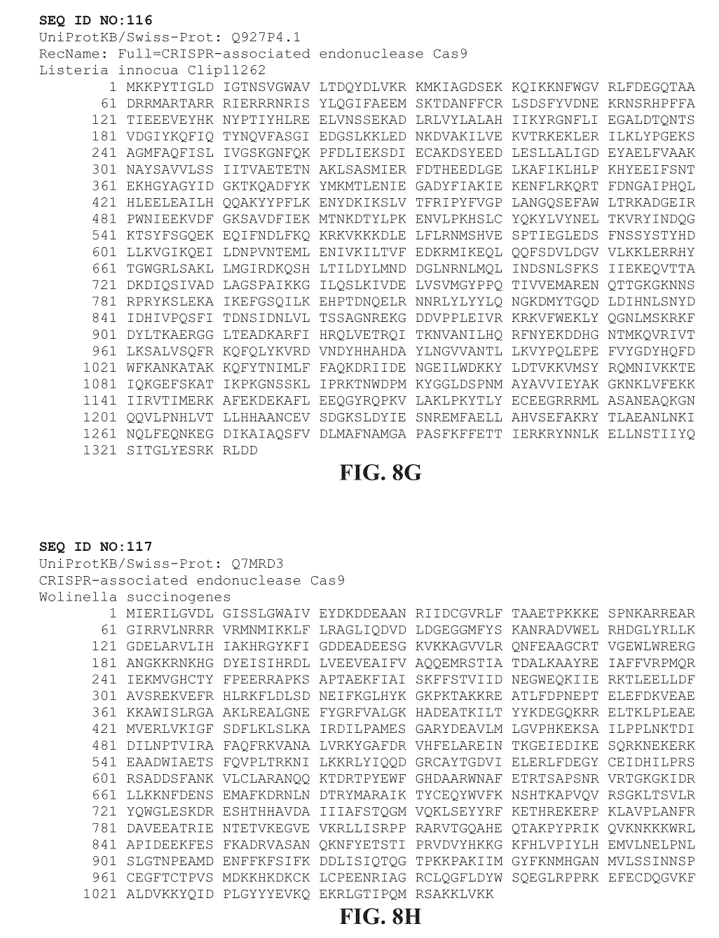

Non-limiting examples of nucleases are described in FIG. 8A-8H. In some embodiments, a vector comprises a regulatory element operably linked to an enzyme-coding sequence encoding a nuclease (e.g., a CRISPR enzyme, such as a Cas protein). Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. pyogenes or S. pneumnoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence.

The nuclease may be a Cas9 homolog or ortholog. In some embodiments, the nuclease is codon-optimized for expression in a eukaryotic cell. In some embodiments, the nuclease directs cleavage of one or two strands at the location of the target sequence. In some embodiments, the nuclease lacks DNA strand cleavage activity. In some embodiments, the first regulatory element is a polymerase III promoter. In some embodiments, the second regulatory element is a polymerase II promoter.

Any useful Cas protein or complex can be employed. Exemplary Cas proteins or complexes include those involved in Type I, Type II, or Type III CRISPR/Cas systems, including but not limited to the CRISPR-associated complex for antiviral defence (Cascade, including a RAMP protein), Cas3 and/or Cas 7 (e.g., for Type I systems, such as Type I-E systems), Cas9 (formerly known as Csn1 or Csx12, e.g., such as in Type II systems), Csm (e.g., in Type III-A systems), Cmr (e.g., in Type III-B systems), Cas10 (e.g., in Type III systems), as well as subassemblies or sub-components thereof and assemblies including such Cas proteins or complexes. Additional Cas proteins and complexes are described in Makarova K S et al., “Evolution and classification of the CRISPR-Cas systems,” Nat. Rev. Microbiol. 2011; 9:467-77, which is incorporated herein by reference in its entirety.

In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In aspects of the invention, nickases may be used for genome editing via homologous recombination. In some instances, the Cas protein includes a modification of one of more of D10A, H840A, N854A, and N863A in SEQ ID NO:110 or in an amino acid sequence sufficiently aligned with SEQ ID NO:110.

As a further example, two or more catalytic domains of Cas9 (RuvC I, RuvC II, and RuvC III) may be mutated to produce a mutated Cas9 substantially lacking all DNA cleavage activity. In some embodiments, a D10A mutation is combined with one or more of H840A, N854A, or N863A mutations to produce a Cas9 enzyme substantially lacking all DNA cleavage activity. In some embodiments, a CRISPR enzyme is considered to substantially lack all DNA cleavage activity when the DNA cleavage activity of the mutated enzyme is less than about 25%, 10%, 5%, 1%, 0.1%, 0.01%, or lower with respect to its non-mutated form. Other mutations may be useful; where the Cas9 or other CRISPR enzyme is from a species other than S. pyogenes, mutations in corresponding amino acids may be made to achieve similar effects.

In some embodiments, the guiding component comprises a modification or sequence that provides for an additional desirable feature (e.g., modified or regulated stability; subcellular targeting; tracking, e.g., a fluorescent label; a binding site for a protein or protein complex; etc.). Non-limiting examples include: a short motif (referred to as the protospacer adjacent motif (PAM)); a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof.

A guiding component and a nuclease can form a complex (i.e., bind via non-covalent interactions). The guiding component provides target specificity to the complex by comprising a nucleotide sequence that is complementary to a sequence of a target sequence. The nuclease of the complex provides the site-specific activity. In other words, the nuclease is guided to a target sequence (e.g., a target sequence in a chromosomal nucleic acid; a target sequence in an extrachromosomal nucleic acid, e.g., an episomal nucleic acid, a minicircle, etc.; a target sequence in a mitochondrial nucleic acid; a target sequence in a chloroplast nucleic acid; a target sequence in a plasmid; etc.) by virtue of its association with the protein-binding segment (e.g., the interacting portion) of the guiding component.

In some embodiments, the guiding component comprises two separate nucleic acid molecules (e.g., a separate targeting portion and a separate interacting portion; a separate first portion and a separate second portion; or a separate targeting portion-first portion that is covalently bound and a separate second portion). In other embodiments, the guiding component is a single nucleic acid molecule including a covalent bond or a linker between each separate portion (e.g., a targeting portion covalently linked to an interacting portion).

FIG. 6C shows an exemplary CRISPR component that includes a guiding component 90 to bind to the target sequence 97, as well as a nuclease 98 (e.g., a Cas nuclease or an endonuclease, such as a Cas endonuclease) that interacts with the guiding component and the target sequence. As can be seen, the guiding component 90 includes a targeting portion 94 configured to bind to the target sequence 97 of a genomic sequence 96 (e.g., a target sequence having substantially complementarity with the genomic sequence or a portion thereof). In this manner, the targeting portion confers specificity to the guiding component, thereby allowing certain target sequences to be activated, inactivated, and/or modified.

The guiding component 90 also includes an interacting portion 95, which in turn is composed of a first portion 91, a second portion 92, and a linker 93 that covalently links the first and second portions. The interacting portion 95 is configured to recruit the nuclease (e.g., a Cas nuclease) in proximity to the site of the target sequence. Thus, the interacting portion includes nucleic acid sequences that provide preferential binding (e.g., specific binding) of the nuclease. Once in proximity, the nuclease 98 can bind and/or cleave the target sequence or a sequence in proximity to the target sequence in a site-specific manner.

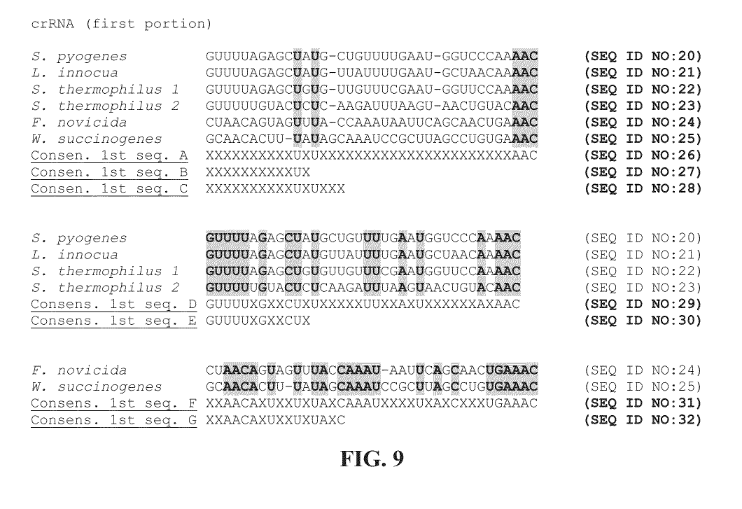

The first portion, second portion, and linker can be derived in any useful manner. In one instance, the first portion can include a crRNA sequence, a consensus sequence derived from known crRNA sequences, a modified crRNA sequence, or an entirely synthetic sequence known to bind a Cas nuclease or determined to competitively bind a Cas nuclease when compared to a known crRNA sequence. Exemplary sequences for a first portion are described in FIG. 9 (SEQ ID NOs:20-32). Another exemplary sequence for a first portion is 5′-GUUUUAGAGCUA-3′ (SEQ ID NO:70). In some embodiments, the first portion is a nucleic acid sequence having at least 80% sequence identity (e.g., at least 85%, 90%, 95%, or 99% sequence identity) to any one of SEQ ID NOs:20-32 and 70 or a complement of any of these, or a fragment thereof (e.g., having a length of about 4, 5, 6, 7, 8, 9, 10, 11, 12, 13, 14, 15, 16, 17, 18, 19, 20, 22, 24, 26, 28, 30, 32, 34, 36, 38, 40, or more nucleotides).

In some embodiments, the first portion is a crRNA sequence that exhibits at least 50%, 60%, 70%, 80%, 90%, 95%, or 99% of sequence complementarity to any one of SEQ ID NOs:20-32 and 70. In other embodiments, the first portion is a fragment (e.g., having a length of about 4, 5, 6, 7, 8, 9, 10, 11, 12, 13, 14, 15, 16, 17, 18, 19, 20, 22, 24, 26, 28, 30, 32, 34, 36, 38, 40, or more nucleotides) of a crRNA sequence that exhibits at least 50%, 60%, 70%, 80%, 90%, 95%, or 99% of sequence complementarity to any one of SEQ ID NOs:20-32 and 70. In one embodiment, the first portion

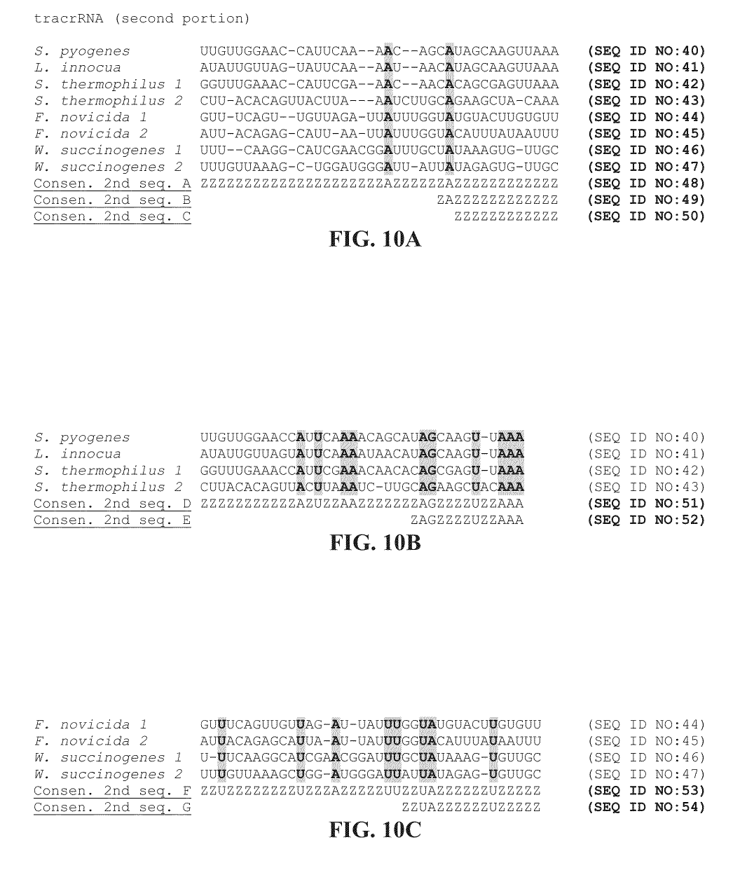

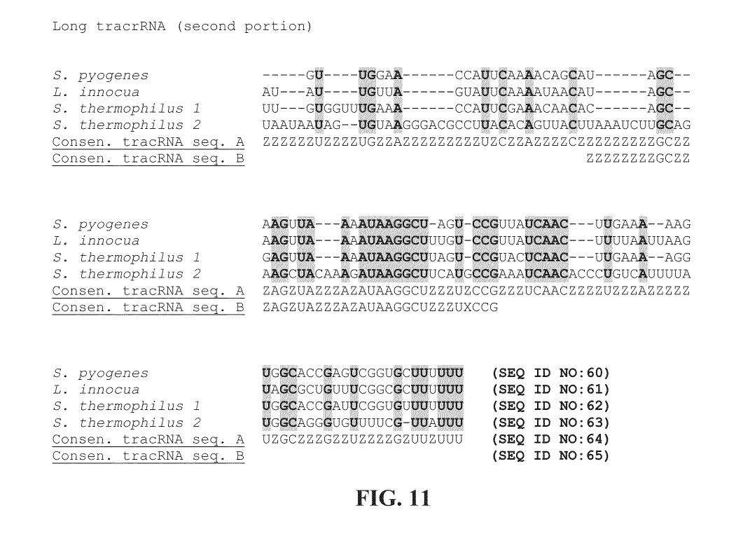

In another instance, the second portion can include a tracrRNA sequence, a consensus sequence derived from known tracrRNA sequences, a modified tracrRNA sequence, or an entirely synthetic sequence known to bind a Cas nuclease or determined to competitively bind a Cas nuclease when compared to a known tracrRNA sequence. Exemplary sequences for a second portion are described in FIG. 10A-10C (SEQ ID NOs:40-54) and in FIG. 11 (SEQ ID NOs:60-65). Another exemplary sequence for a second portion is 5′-UAGCAAGUUAAAAUAAGGCUAGUCCG-3′ (SEQ ID NO:71).

In some embodiments, the second portion is a nucleic acid sequence having at least 80% sequence identity (e.g., at least 85%, 90%, 95%, or 99% sequence identity) to any one of SEQ ID NOs:40-54, 60-65, and 71 or a complement of any of these, or a fragment thereof (e.g., having a length of about 4, 5, 6, 7, 8, 9, 10, 11, 12, 13, 14, 15, 16, 17, 18, 19, 20, 22, 24, 26, 28, 30, 32, 34, 36, 38, 40, or more nucleotides).

In some embodiments, the second portion is a tracrRNA sequence that exhibits at least 50%, 60%, 70%, 80%, 90%, 95%, or 99% of sequence complementarity to any one of SEQ ID NOs:40-54, 60-65, and 71. In other embodiments, the second portion is a fragment (e.g., having a length of about 4, 5, 6, 7, 8, 9, 10, 1, 12, 13, 14, 15, 16, 17, 18, 19, 20, 22, 24, 26, 28, 30, 32, 34, 36, 38, 40, or more nucleotides) of a tracrRNA sequence that exhibits at least 50%, 60%, 70%, 80%, 90%, 95%, or 99% of sequence complementarity to any one of SEQ ID NOs:40-54, 60-65, and 71.

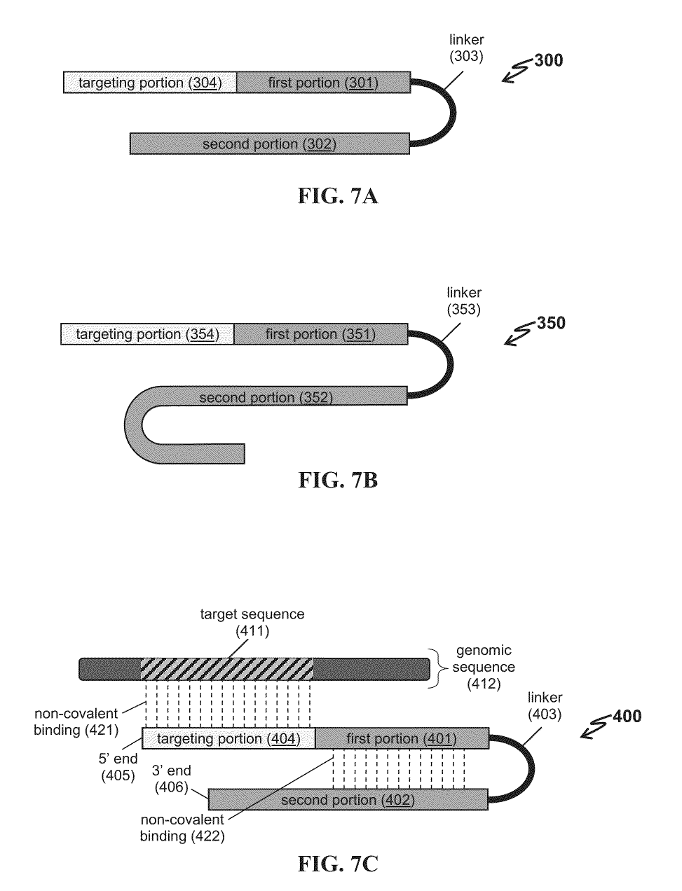

The linker can be any useful linker (e.g., including one or more transcribable elements, such as a nucleotide or a nucleic acid, or including one or more chemical linkers). Further, the linker can be derived from a fragment of any useful tracrRNA sequence (e.g., any described herein). The first and second portions can interact in any useful manner. For example, the first portion can have a sequence portion that is sufficiently complementary to a sequence portion of the second portion, thereby facilitating duplex formation or non-covalent bonding between the first and second portion. In another example, the second portion can include a first sequence portion that is sufficiently complementary to a second sequence portion, thereby facilitating hairpin formation within the second portion. Further CRISPR components are described in FIG. 7A-7C.

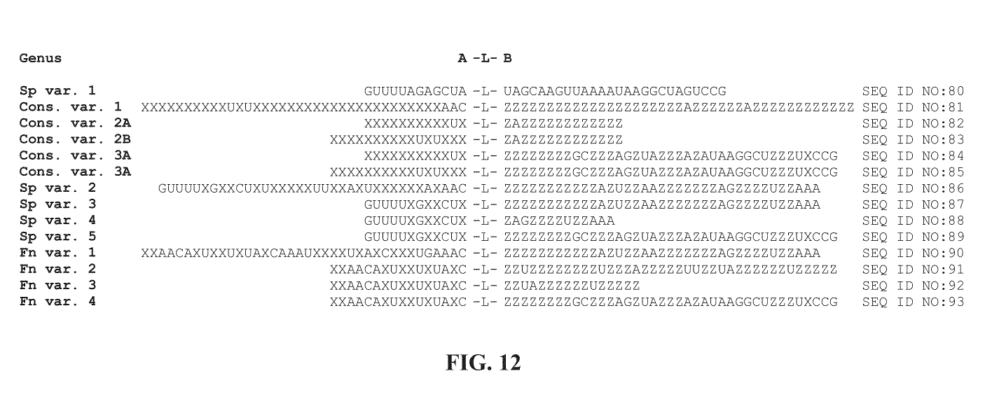

In another embodiment, the guiding component has a structure of A-L-B, in which A includes a first portion (e.g., any one of SEQ ID NOs:20-32 and 70, or a fragment thereof), L is a linker (e.g., a covalent bond, a nucleic acid sequence, a fragment of any one of SEQ ID NOs:40-54, 60-65, and 71, or any other useful linker), and B is a second portion (e.g., any one of SEQ ID NOs:40-54, 60-65, and 71, or a fragment thereof) (FIG. 12). In another embodiment, the guiding component is a sequence having at least 80% sequence identity (e.g., at least 85%, 90%, 95%, or 99% sequence identity) to any one SEQ ID NOs:80-93, or a fragment thereof.

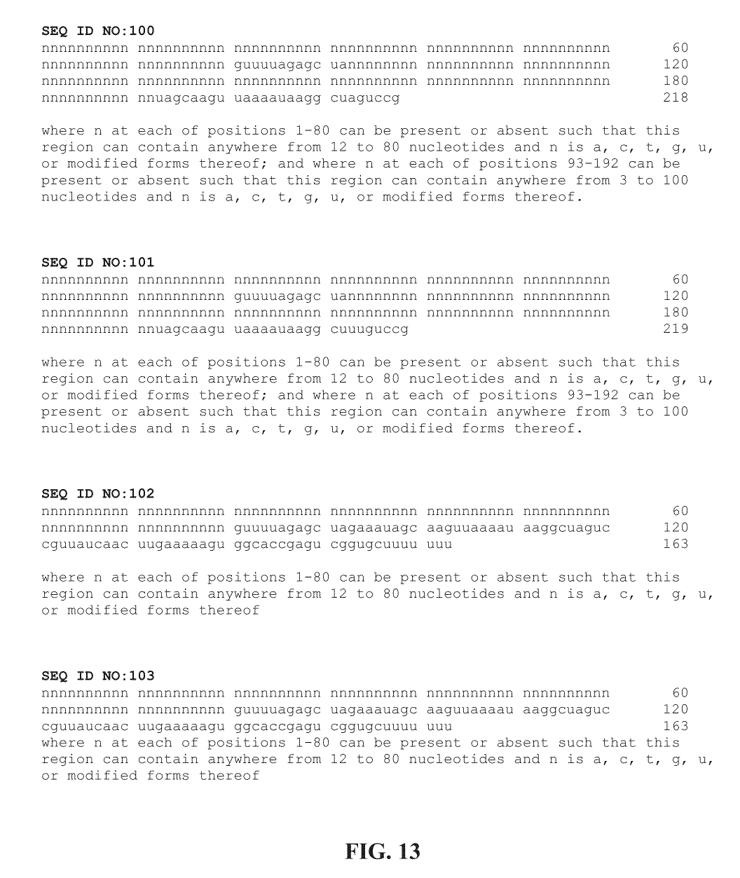

In yet another embodiment, the guiding component is a sequence that exhibits at least 50%, 60%, 70%, 80%, 90%, 95%, or 99% of sequence complementarity to any one SEQ ID NOs:100-103, or a fragment thereof (FIG. 13). In another embodiment, the guiding component is a sequence having at least 80% sequence identity (e.g., at least 85%, 90%, 95%, or 99% sequence identity) to any one SEQ ID NOs: 100-103, or a fragment thereof.

FIG. 1D shows delivery of a CRISPR component (e.g., as a plasmid) by employing a silica carrier. The CRISPR components can be provided in any useful form (e.g., a vector for in vivo expression, a phage, a plasmid, etc.). In some embodiments, the CRISPR component includes ds plasmid DNA, which is modified to express RNA and/or a protein. In other embodiments, the CRISPR component is supercoiled and/or packaged (e.g., within a complex, such as those containing histones, lipids (e.g., lipoplexes), proteins (e.g., cationic proteins), cationic carrier, nanoparticles (e.g., gold or metal nanoparticles), etc.), which may be optionally modified with a nuclear localization sequence (e.g., a peptide sequence incorporated or otherwise crosslinked into histone proteins, which comprise the histone-packaged supercoiled plasmid DNA). Other exemplary histone proteins include H1, H2A, H2B, H3 and H4, e.g., in a ratio of 1:2:2:2:2 with optional nuclear localization sequences (e.g., any described herein, such as SEQ ID NOs:9-12).

The CRISPR component can include any useful promoter sequence(s), expression control sequence(s) that controls and regulates the transcription and translation of another DNA sequence, and signal sequence(s) that encodes a signal peptide. The promoter sequence can include a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation, as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgamo sequences in addition to the −10 and −35 consensus sequences.

In addition, the CRISPR components can be formed from any useful combination of one or more nucleic acids (or a polymer of nucleic acids, such as a polynucleotide). Exemplary nucleic acids or polynucleotides of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids, chimeras, or modified forms thereof. Exemplary modifications include any useful modification, such as to the sugar, the nucleobase, or the internucleoside linkage (e.g., to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications according to the present invention may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

Toxicity of CRISPR components, to the host, can be minimized in any useful manner. For instance, toxicity can result from protocells or carriers due to expression of Cas9 products or immune responses. Specifically, the lifetime of CRISPR components in the cell can be controlled by adding features that are stabilized or destabilized with cellular proteases, by inducing expression only under a microbial or viral promoter, and by using guiding components with modified backbones (e.g., 2-OMe) to minimize immune recognition.

Resistance to CRISPR components can be minimized. Any single antibiotic or antiviral countermeasure is prone to the development of resistance, so pathogens will likely mutate around individual guiding component targets. However, we will prevent the development of resistance by targeting orthogonal mechanisms via multiplexed guiding components in combination with current antivirals/antimicrobials.

Off-target mutations or genetic modification can be minimized. For instance, bioinformatic guiding component design programs can be used to determine minimal effective CRISPR component doses. If needed, the nickase version of Cas9 can be employed.

The CRISPR component can be employed to target any useful nucleic acid sequence (e.g., present in the host's genomic sequence and/or the pathogen's genomic sequence). In one instance, the target sequence can include a sequence present in the host's genomic sequence in order, e.g., activate, inactive, or modify expression of factor or proteins within the host's cellular machinery. For instance, the target sequence can bind to one or more genomic sequences for an immunostimulatory protein that, upon expression, would enhance the immune response by the host to an infection. Pathogens are known to down-regulate proteins that would otherwise assist in recognizing non-self protein motifs. Thus, in another instance, the target sequence can bind to one or more regulator proteins and enhance their transcription and expression. In yet another instance, one or more polypeptides may be up-regulated, as compared to the normal basal rate, and such up-regulation may be modified by the presence of the pathogen. Accordingly, the target sequence can be employed to bind to one or more up-regulated polypeptides in order to inactivate or repress transcription/expression of those polypeptides.

In yet another instance, the target sequence can be employed to activate, inhibit, and/or modify a target sequence. For instance, the target sequence can be configured to activate one or more target sequences encoding proteins that promote programmed cell death or apoptosis.

The CRISPR component can be employed to activate the target sequence (e.g., the Cas polypeptide can include one or more transcriptional activation domains, which upon binding of the Cas polypeptide to the target sequence, results in enhanced transcription and/or expression of the target sequence), inactivate the target sequence (e.g., the Cas polypeptide can bind to the target sequence, thereby inhibiting expression of one or more proteins encoded by the target sequence; the Cas polypeptide can introduce double-stranded or single-stranded breaks in the target sequence, thereby inactivating the gene; or the Cas polypeptide can include one or more transcriptional repressor domains, which upon binding of the Cas polypeptide to the target sequence, results in reduced transcription and/or expression of the target sequence), and/or modify the target sequence (e.g., the Cas polypeptide can cleave the target sequence of the pathogen and optionally inserts a further nucleic acid sequence).

Any useful transcriptional activation domains can be employed (e.g., VP64, VP16, HIV TAT, or a p65 subunit of nuclear factor KB). In particular, such activation domains are useful when employed with a deactivated or modified form of the Cas polypeptide with minimized cleavage activity. In this way, specific recruitment of the Cas polypeptide to the target sequence is enabled by the interacting portion of the target component, and transcriptional activity is controlled by the activation domains.

Further, any useful transcriptional repressor domains can be employed (e.g., a Krüppel-associated box domain, a SID domain, an Engrailed repression domain (EnR), or a SID4X domain). In particular, such repressor domains can be employed with a deactivated or modified form of the Cas polypeptide with minimized cleavage activity or with an active Cas polypeptide with retained endonuclease activity.

A guiding component may be selected to target any target sequence. In some embodiments, the target sequence is a sequence within a genome of a host (e.g., a host cell) or a pathogen (e.g., a pathogen cell). In some embodiments, the guiding component is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guiding component is less than about 75, 50, 45, 40, 35, 30, 25, 20, 15, 12, or fewer nucleotides in length. The ability of a guiding component to direct sequence-specific binding of a CRISPR complex to a target sequence may be assessed by any suitable assay. For example, the components of a CRISPR system sufficient to form a CRISPR complex, including the guiding component to be tested, may be provided to a host cell having the corresponding target sequence, such as by transfection with vectors encoding the components of the CRISPR sequence, followed by an assessment of preferential cleavage within the target sequence, such as by Surveyor assay. Similarly, cleavage of a target sequence may be evaluated in a test tube by providing the target sequence, components of a CRISPR complex, including the guiding component to be tested and a control guiding component different from the test guiding component, and comparing binding or rate of cleavage at the target sequence between the test and control guiding component reactions. Other assays are possible, and will occur to those skilled in the art.

Algae and Plants

The present invention can be employed to manipulate any useful host or sample, including algal samples and plant samples. The delivery platform can be employed in any useful manner. The present delivery platform can be adapted to recognize the target and, if needed, deliver the one or more cargos to treat that target.

The algae can include any useful organism, such as chlorophyta, diatoms, plankton, protists, and/or cyanobacteria. For instance, algae can include one or more photosynthetic organisms, including one or more microalgae, macroalgae, diatoms, green algae, yellow algae, phytoplankton, plankton, haptophytes, and/or cyanobacteria. Exemplary algae include Achnanthes, Ankistrodesmus (e.g., A. falcatus or A. fusiformis), Aphanizomenon, Arthrospira (e.g., A. maxima), Bacillariophyceae, Botryococcus (e.g., B. braunii), Chlamydocapsa (e.g., C. bacillus), Chlamydomonas (e.g., C. perigranulata or C. reinhardtii), Chlorella (e.g., C. marina, C. vulgaris, C. variabilis, C. sorokiniana, C. minutissima, or C. pyrenoidosa), Chlorococcum (e.g., C. infusionum, C. littorale, or C. humicola), Chlorogloeopsis (e.g., C. fritschii), Chlorophyceae, Chrysophyceae, Cyanophyceae, Dunaliella (e.g., D. bardawil, D. bioculata, D. primnolecta, D. tertiolecta, or D. salina), Ellipsoidion, Haematococcus (e.g., H. pluvialis), Isochrysis, Kirchneriella (e.g., K. lunaris), Nannochloropsis (e.g., N. salina, N. gaditana, or N. oculata), Neochloris (e.g., N. oleoabundans), Nitzschia, Ostreococcus (e.g., O. tauri, O. lucinmarinus. O. mediterraneus, and O. spp. RCC809), Phaeodactylum (e.g., P. tricornutum), Porphyridium (e.g., P. purpureum), Pyrmnesium (e.g., P. parvum), Scenedesmus (e.g., S. obliquus, S. quadricauda, or S. dimorphus), Schizochytrium, Skeletonema (e.g., S. costatum), Spirogyra, Spirulina (e.g., S. maxima or S. platensis), Synechococcus (e.g., S. elongatus), Tetraselmis (e.g., T. maculata or T. suecica), and/or Thalassiosira (e.g., T. pseudonana). Additional algae species and organisms are described in Schneider R C S et al., “Potential production of biofuel from microalgae biomass produced in wastewater,” in Biodiesel-Feedstocks, Production and Applications, Prof. Zhen Fang (ed.), InTech, 2012, 22 pp., which is incorporated herein by reference in its entirety.

Algae can be grown in any useful manner. For instance, the algae can be provided as a monoculture or as a polyculture (e.g., a polyculture turf biomass or benthic algal polyculture turf) grown in a pond, a bioreactor, a field plate, a tank reactor, etc. In addition, the algae can be derived from or grown within any source, including wastewater (e.g., agribusiness, municipal, and/or industrial wastewater), as well as water bodies with excess nutrients. Biomass from high productivity polyculture sources, such as those used for waste water treatment, commonly contain 20-50% protein, 20-40% carbohydrates, 5-20% lipids, and up to 50% ash.

A plant refers to whole plants (e.g., immature or mature whole plants), plant organs (e.g., leaves, stems, buds, flowers, roots, root tips, anthers, seed, grain, embryo, pollen, ovules, cotyledons, hypocotyls, pods, shoots, stalks, etc.), and plant cells (including tissues, tissue cultures, cell, etc.), and progeny of same. Exemplary plants include those amenable to transformation techniques, including both monocotyledonous and dicotyledonous plants, as well as certain lower plants such as algae. Suitable plants include plants of a variety of ploidy levels, including polyploid, diploid, and haploid. Non-limiting plants include tobacco, maize, pea, canola, Indian mustard, millet, sunflower, hemp, switchgrass, duckweed, sugarcane, sorghum, and sugar beet.

Using the delivery vehicle described herein, the plant can be transformed into a transgenic plant, i.e., a plant that comprises within its cells an exogenous polynucleotide. The term “transgenic” as used herein does not encompass the alteration of the genome (chromosomal or extra-chromosomal) by conventional plant breeding methods (e.g., crosses) or by naturally occurring events such as random cross-fertilization, non-recombinant viral infection, non-recombinant bacterial transformation, non-recombinant transposition, or spontaneous mutation.

Compositions

The present invention also includes a pharmaceutical composition including an effective amount of a delivery platform (e.g., any described herein). In some instances, the pharmaceutical composition includes a population of particles (e.g., any described herein) in an amount effective for modulating or modifying the plant or alga in combination with a pharmaceutically acceptable carrier, additive, or excipient. In other instances, the composition further includes a drug, an agrochemical, a nutrient, etc., which is not disposed as cargo within the particle.

The composition can be formulated in any useful manner with a plurality of delivery platforms (e.g., plurality or population of particles). Such formulations can be included with any useful medium, excipient (e.g., lactose, saccharide, carbohydrate, mannitol, leucine, PEG, trehalose, etc.), additive, propellant, solution (e.g., aqueous solution, such as a buffer). In one instance, the composition includes an aerosolized formulation, a liquid formulation, or a powdered formulation. The delivery platform can have any useful dimension (e.g., a mean particle size is of from about 2 to about 5 μm, or any described herein), colloidal stability, functionalization, surface charge, etc., for use in the formulation.

The present invention also relates to a composition including an effective amount of a plurality (e.g., a population) of particles (e.g., carriers and/or protocells) and an acceptable additive, excipient, preservative, or solution (e.g., an aqueous solution).

Liquid compositions or formulations can be prepared by dissolving or dispersing the population of MSNPs, protocells, and/or carriers (about 0.5% to about 20% by weight or more), and optional pharmaceutical adjuvants, in a carrier, such as, for example, aqueous saline, aqueous dextrose, glycerol, or ethanol, to form a solution or suspension.

When the composition is employed in the form of solid preparations, the preparations may be tablets, granules, powders, capsules, or the like. In a tablet formulation, the composition is typically formulated with additives, e.g., an excipient such as a saccharide or cellulose preparation, a binder such as starch paste or methyl cellulose, a filler, a disintegrator, and other additives typically used in the manufacture of medical preparations.

Methods for Modulating a Target Sequence

The delivery platform can be configured to bind to a target sequence in a genomic sequence of the subject (e.g., a plant or an alga) in order to modulate that target sequence. Modulation can include activating, inactivating, deactivating, and/or modifying expression or activity of the target sequence. For example, the cargo or the biological package can bind to the target sequence, e.g., thereby inhibiting expression of one or more proteins encoded by the target sequence. In another example, the cargo or the biological package carrier cleaves the target sequence and optionally inserts a further nucleic acid sequence into the genomic sequence of the subject. In yet another example, the cargo or the biological package carrier activates the target sequence.

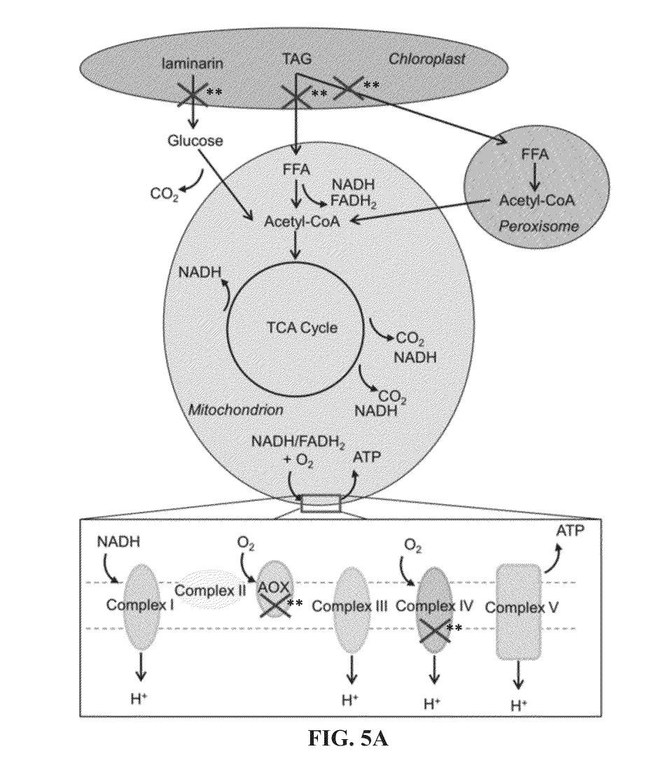

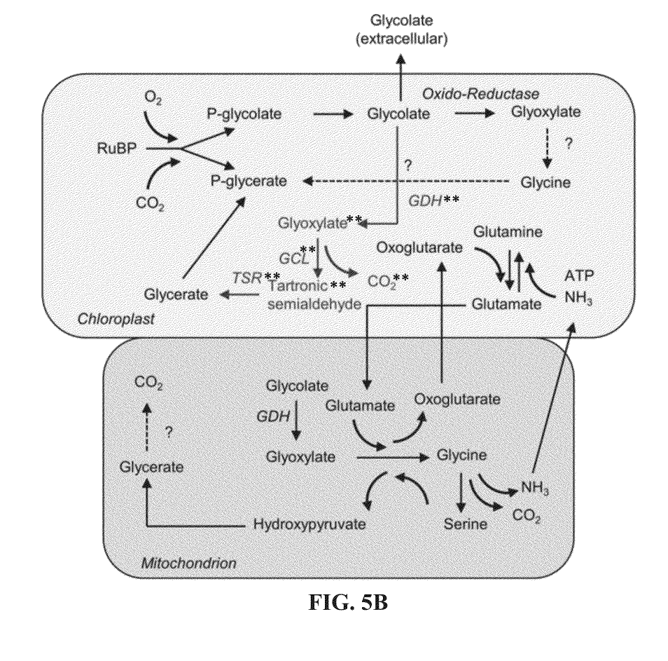

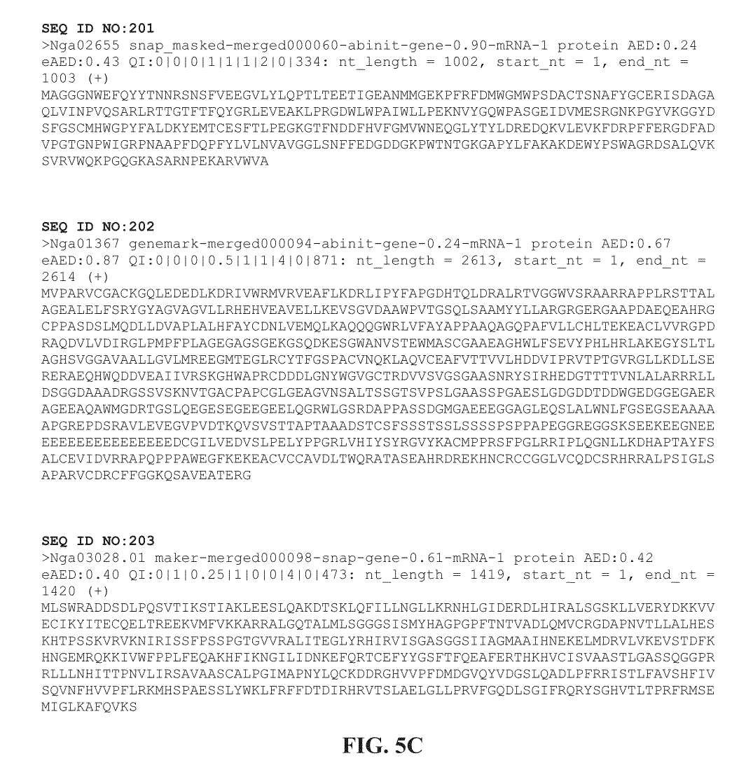

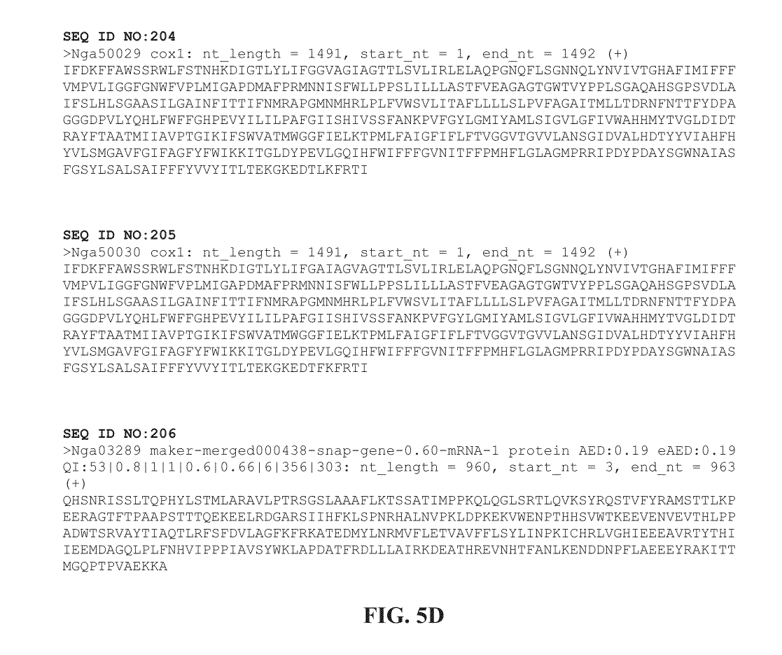

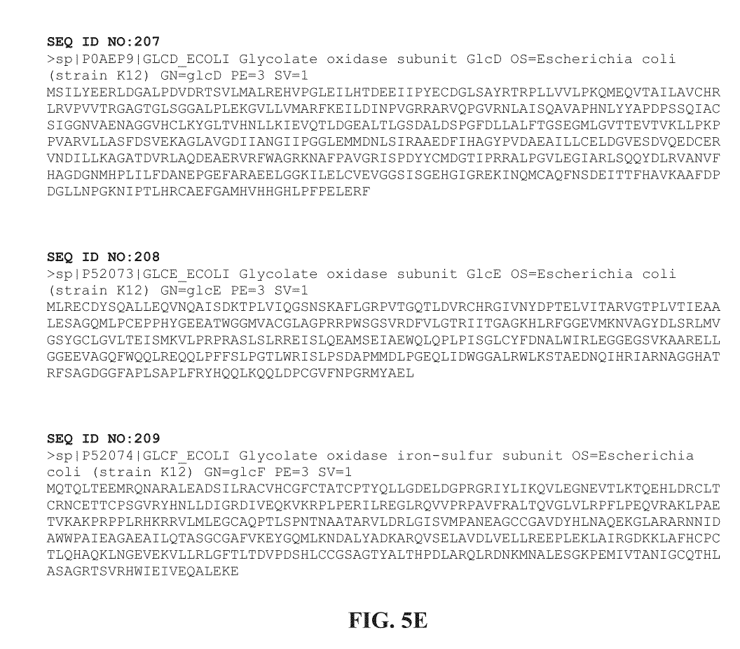

Any useful target sequence can be modulated. Exemplary target sequences include those that limit nighttime loss of biomass due to dark respiration (e.g., a nucleic acid that encodes for any polypeptide in FIG. 5A-5B), as well as target sequences that encode a polypeptide having at least 80% sequence identity (e.g., at least about 85%, 90%, 95%, 96%, 97%, 98%, 99%, 99.5%, or 99.9%) to any one of SEQ ID NOs:201-209 (FIG. 5C-5E) or a fragment thereof.

Uses

The delivery platform can be employed to provide an improved plant or alga, which in turn can be further processed to provide any useful product (e.g., a biofuel including biodiesel or bioethanoal, a biomass, a lipid, a co-product, a feed, a fertilizer, a pharmaceutical intermediate, and other useful building blocks).

A plant or algal biomass can be incubated with nutrient-loaded water and sunlight to promote growth, and then harvested. Typically, an algal biomass will include equal fractions of proteins, carbohydrates, and lipids (collectively, biocomponents). Further treatment steps can be employed to breakdown these biocomponents of the plant or algal biomass and release useful residuals.

Lipids from the biomass can be captured by distillation, by lipid disruption, by osmotic stress, by mechanical disruption, and/or by solvent co-extraction. Lipids, including lipid vesicles and microparticles, can be extracted by lipophilic solvents, such as hexane and ethyl acetate, avoiding high energy fractional distillation of the >C2 alcohol and lipid products. Any useful distillation and extraction techniques can be employed, including flash extraction, ionic liquid extraction, etc., to isolate one or more biocrude oil, aqueous phases, aqueous co-products, nutrients, etc.

Phase separation steps can be employed to separate components of a liquefied mixture, fermentation broth, aqueous fraction, a non-aqueous fraction, alcohol fraction, etc. Such steps include any that separate liquid from solid phases, as well as separate two or more phases that can be differentiated based on solubility, miscibility, etc. (e.g., as those present in non-aqueous phases, aqueous phases, lipophilic phases, etc.) in any useful solvent (e.g., an organic solvent, an aqueous solvent, water, buffer, etc.). Phase separation techniques include flash separation (e.g., separation of a fraction into biocrude oil, biocomponents, lipids, solid residuals, aqueous phase, and/or aqueous co-products), acid absorption (e.g., absorption of acid in a matrix to provide recovered nutrients and water for recycled use), filtration, distillation, solvent extraction, ion liquid extraction, etc. The resultant products and co-products can include one or more intermediate products that can optionally be processed to form useful end-use products.

Hydrotreatment is generally used to convert compositions (e.g., including any useful fraction of the plant or algal biomass) into useful intermediate products or end-use products. Such hydrotreatment generally includes use of high temperatures to institute any useful chemical change, e.g., to break apart triglycerides; to form low molecular weight carbon species, such as optionally substituted alkanes, cycloalkanes, or aryls; to saturate carbon chains with hydrogen; to denitrogenate species; and/or to deoxygenate species to form alkanes, such as n-alkanes.

Hydrotreatment can include isomerization, hydrocracking, distillation, hydrodeoxygenation, catalytic processing (e.g., such as use of one or more catalysts to remove nitrogen, oxygen, and/or sulfur from a fraction under any useful condition, such as a pressure of from about 5 MPa to about 15 MPa and a temperature of from about 200° C. to about 450° C.), liquefaction (e.g., such as hydrothermal liquefaction (HTL) or catalytic liquefaction of one or more lipids into a biofuel or a biofuel intermediate by use of an operating temperature of from about 100° C. to about 500° C.), transesterification (e.g., treatment of one or more lipids with an alcohol and an optional catalyst to produce methyl ester biodiesel), and/or catalytic hydrothermal gasification (CHG) (e.g., of an aqueous co-product into biogas).

The hydrotreatment process can employ any useful catalyst (e.g., a metal catalyst, such a copper-based catalyst (e.g., CuCr, CuO), a nickel-based catalyst (e.g., NiMo), a ruthenium-based catalyst, a palladium-based catalyst (e.g., Pd/C), a platinum-based catalyst, a rhenium-based catalyst, or a cobalt-based catalyst (e.g., CoMo)) in the presence of any carrier (e.g., a zeolite, an alumina, etc.); any useful reagent, such as hydrogen (e.g., H₂) or water (e.g., supercritical water); any useful pressure, e.g., such as from about 3 MPa to about 30 MPa (e.g., from about 5 MPa to about 20 MPa); and/or any useful temperature, e.g., such as from about 100° C. to about 500° C. (e.g., from about 250° C. to about 350° C.). Further exemplary hydrotreatment conditions are described in Ma F et al., “Biodiesel production: a review,” Bioresource Technol. 1999; 70:1-15; Tran N H et al., “Catalytic upgrading of biorefinery oil from micro-algae,” Fuels 2010; 89:265-74; and Wildschut J et al., “Catalyst studies on the hydrotreatment of fast pyrolysis oil,” Appl. Catalysis B 2010; 99:298-306, each of which is incorporated herein by reference in its entirety.

Exemplary biofuels formed by hydrotreatment include naphtha, biodiesel (e.g., including one or more unsaturated fatty acids or fatty acid esters, such as of from about 10% to about 35% of a long chain fatty acid having a C₁₃-C₂₁ tail, such as a palmitic fatty acid (C₁₆ tail), linoleic fatty acid (C₁₈ tail), oleic fatty acid (C₁₈ tail), and/or stearic fatty acid (C₁₈ tail)), green diesel, renewable aviation fuel, hydrocarbons (e.g., light hydrocarbons), alcohol (e.g., ethanol; propanol, such as 1-propanol; butanol, such as n-butanol, isobutanol, 2-butanol, 3-butanol, 2-methyl-1-butanol, 3-methyl-1-butanol, etc.), and/or a biogas (e.g., hydrogen or methane). Other products formed by hydrotreatment include solid residuals (e.g., biochar and ash), aqueous co-products (e.g., ketoacids, amines, nutrients, etc.), as well as other useful co-products (e.g., animal feed, fertilizer, glycerine, biopolymers, etc.).

### EXAMPLES

### Example 1

### Algal Targets

The NanoCRISPR delivery platform is widely applicable to any useful target that would benefit from exogenous genetic modification. For instance, the NanoCRISPR platform can be adapted to target industrially-relevant algal strains for biofuel applications. In another instance, the platform can be employed to genetically knock out proteins that are detrimental for efficient algal growth (e.g., use of a genomic target sequence (e.g., gRNA) that target and cleaves the DNA sequence encoding for an enzyme or cofactor that reduces algal growth).

Algal biofuels are promising candidates for renewable energy, yet current algal productivities must be improved by 2- to 5-fold to achieve the rates necessary for economically-feasible fuel production. However, algal cell walls are particularly recalcitrant, thwarting the delivery of DNA for genetic engineering efforts to improve productivity. As a result, only one model strain of algae, Chlamydomonas reinhardtii is currently able to be consistently engineered, while the more industrially-relevant strains can only be improved by random mutagenesis.

The nanoparticle delivery methods described herein may overcome this barrier, which is currently preventing the rational development of industrial strains of algae. To assess this potential application, we will assess the ability of NanoCRISPRs designed for bacterial uptake to penetrate several industrially-relevant algal strains. If successful, this would enable rational strain development for algal biofuels. Typical examples of rational strain development in other organisms yield approximately 2-fold improvements in yield after only one engineering effort, with a 60-fold reduction in strain development time compared to similar improvements achieved via random mutagenesis (see, e.g., Thykaer J et al., Metab. Eng. 2003; 5:56-69). Hence, multiple rounds of genetic engineering should enable algal productivities to reach the 2022 target set forth by the Dept. of Energy's Office of Energy Efficiency and Renewable Energy of 5,000 gallons of biofuel feedstock per acre per year.

### Example 2

### Nanoparticle Tools for the Domestication of Algae and Plants

Microalgae are ideal candidates as synthetic biology chasses for complex settings. These robust, photosynthetic microorganisms are capable of surviving under a range of environmental conditions and require minimal nutrients for growth (see, e.g., Rothschild L J et al., “Life in extreme environments,” Nature 2001; 409(6823): 1092-101). Despite these fundamental advantages, eukaryotic microalgae remain largely undomesticated due to transformation limitations associated with hardy algal cell walls and the lack of effective tools for targeted genetic modification (see, e.g., León-Bañares R et al., “Transgenic microalgae as green cell-factories,” Trends Biotechnol. 2004; 22(1):45-52). The platform herein can be employed to develop effective tools for the domestication of algae and plants.

We propose to develop tools for targeted genetic modification of eukaryotic microalgae by combining nanoparticle-mediated transformation methods with CRISPR-Cas9 technology for genome editing, thereby providing a NanoCRISPR platform. The particle delivery platform described herein provide unique control over the properties of the MSNP core, shell, and SLB, which can be independently modulated to tailor loading and release of physicochemically disparate biological packages and/or cargos, as well as time-dependent biodistribution and biodegradation.

In prior work, we have demonstrated efficient delivery of nucleic acid and enzyme cargos into mammalian cells and bacteria using porous silica nanoparticles encased in a lipid layer, such as protocells or silica carriers (see, e.g., Ashley C E et al., “The targeted delivery of multicomponent cargos to cancer cells by nanoporous particle-supported lipid bilayers,” Nat. Mater. 2011; 10(5):389-97; Ashley C E et al., “Delivery of small interfering RNA by peptide-targeted mesoporous silica nanoparticle-supported lipid bilayers,” ACS Nano 2012; 6(3):2174-88; and Epler K et al., “Delivery of ricin toxin a-chain by peptide-targeted mesoporous silica nanoparticle-supported lipid bilayers,” Adv. Healthc. Mater. 2012 May; 1(3):348-53). Such delivery platforms have 100 to 10,000-fold higher loading capacities than other nanoparticle delivery vehicles, stabilize encapsulated cargo molecules over a range of temperatures, promote efficient uptake by target cells, and enable controlled, intracellular release of the cargo. This delivery approach can be modified for efficient transformation of a wide range of microalgal species with varying cell size and cell wall composition. We will tune nanoparticle size, surface charge, and biological modification (e.g., cell penetrating peptides) to optimize uptake.

After establishing efficient, nanoparticle-mediated transformation methods, we will utilize this technology for delivery of CRISPR-Cas9 components for targeted genetic modification of algal nuclear genomes. Clustered, Regularly Interspaced, Short Palindromic Repeats (CRISPR)-Cas9, initially studied as a ‘bacterial immune system’, has been exploited for targeted genetic modification of a wide range of eukaryotic species (see, e.g., Sander J D et al., “CRISPR-Cas systems for editing, regulating and targeting genomes,” Nat Biotech. 2014; 32(4):347-55).

In some embodiments, the CRISPR-Cas9 technology includes two components: a Cas9 enzyme that cleaves double-stranded DNA, and the guide RNA (gRNA) that includes a region homologous to the target DNA sequence and a region that recruits Cas9. The double-stranded break at the target site often leads to mutation (i.e., gene knockout) and has been demonstrated to promote DNA fragment insertion at the target site (i.e., targeted genome integration). The proposed nanoparticle-mediated CRISPR-Cas9 tools will improve upon traditional CRISPR-Cas9 technology by (1) enhancing uptake; (2) eliminating the requirement for host expression of Cas9 and gRNA, as these components can be synthesized in domesticated laboratory strains, isolated, and delivered via nanoparticles; (3) providing both stability and high packing of the Cas9 and gRNA components; (4) reducing off-target effects via transient Cas9 and gRNA; and (5) enabling an efficient, one-step process for targeted genetic modification in a wide range of undomesticated eukaryotic hosts.

The platform herein can enable targeted genetic modification of a diverse range of undomesticated algal species, directly impacting a wide range of applications. Enhanced cell membrane penetration resulting from the nanoparticle delivery method may enable successful transformation of other undomesticated organisms with tough cell walls. Additionally, the ability to deliver active Cas9 and gRNA directly to the cell for genetic modification will greatly accelerate traditional CRISPR-Cas9 methods, as genetic modification will not require a priori knowledge of the host's genetic regulatory mechanisms for Cas9 and gRNA expression. The inherent transient nature of Cas9 and gRNA in the host cell will also minimize off-target genetic modifications, a significant concern with traditional CRISPR-Cas9 technology. Subsequently, this research may indirectly impact standard genetic modification techniques for all eukaryotes.

Direct applications of this technology include the genetic modification of algae for aquatic biosensors, optimized algae for wastewater treatment and other bioremediation applications in complex settings, metabolic pathway optimization for bioenergy applications, and genetic modification of algae for the production of high-value chemical products. Additionally, the proposed nanoparticle-mediated CRISPR-Cas9 technology may enable in situ genetic modification of natural microbial communities, as this is a one-step process that does not require electroporation, gene guns, or other laboratory transformation equipment. A rather unexplored application may be the use of nanoparticle-mediated CRISPR-Cas9 for ‘ecological engineering’. As the primary producers in aquatic food webs and also responsible for nearly 50% of the current CO₂ fixation on Earth, the genetic modification of algae for enhanced CO₂ fixation may help to address climate change concerns. Of course, the potential ecological ramifications of releasing modified algae into the environment must first be assessed. As with any method of genetic modification, the proposed tools may also be used for nefarious purposes, such as the introduction of toxin-producing genes or pathogenic elements. The ability to introduce such elements by simply dropping a CRISPR-Cas9 nanoparticle into the environment is also a dual-use concern for the proposed technology.

The proposed approach combines two recently developed technologies, nanoparticle delivery and CRISPR-Cas9 genome editing, to enable targeted genetic modification of undomesticated algal species. Two historical factors limiting algal domestication are the challenge of penetrating tough algal cell walls to enable transformation and the lack of available tools for targeted genome editing. Recent advancements in CRISPR-Cas9 technology have demonstrated that these genome editing tools can be used for targeted modification in a wide range of eukaryotic hosts (see, e.g., Sander J D et al., Nat Biotech. 2014; 32(4):347-55).

Nanoparticle delivery of nucleic acids and proteins to mammalian cells and bacteria was recently established by Sandia National Laboratories (see, e.g., Ashley C E et al., “The targeted delivery of multicomponent cargos to cancer cells by nanoporous particle-supported lipid bilayers,” Nat. Mater. 2011; 10:389-97), and nanoparticle-mediated delivery of nucleic acids to plant cells (see, e.g., Torney F et al., “Mesoporous silica nanoparticles deliver DNA and chemicals into plants,” Nat. Nanotechnol. 2007; 2(5):295-300) suggests that this delivery method may overcome transformation limitations in algae as well. The combination of these two revolutionary technologies, CRISPR-Cas9 for genome editing and nanoparticle-mediated delivery, is likely to enable algal domestication across a wide range of species where previous technologies have either failed or remain limited to a single species. The proposed nanoparticle-mediated delivery also enables new frontiers to be explored for the employment of CRISPR-Cas9 technology. The potential to deliver active Cas9 and gRNA via nanoparticle packaging will enable more efficient genome editing compared to traditional plasmid-mediated approaches which require host expression of both Cas9 and gRNA.

Key technical challenges in the proposed study include achieving efficient nanoparticle uptake across algal species with varying cell wall properties, packaging and delivery of active Cas9 enzyme and gRNA, and overcoming gene silencing mechanisms known to be active in many algal species. While mammalian cells uptake nanoparticles via endocytosis, there is only limited evidence that such endocytic pathways are active in algae (see, e.g., Battey N H et al., “Exocytosis and endocytosis,” Plant Cell 1999; 11:643-59). Therefore, we propose to investigate various nanoparticle sizes, surface charges, and even protein modifications to promote efficient uptake in algae.

Nanoparticle delivery of active Cas9 enzyme and gRNA will require that the synthesis process does not denature the enzyme nor degrade the gRNA. We have previously demonstrated that the delivery platforms stabilize encapsulated enzymes and nucleic acids (see, e.g., Ashley C E et al., ACS Nano 2012; 6(3):2174-88; and Epler K et al., Adv. Healthc. Mater. 2012 May; 1(3):348-53). Lastly, if active Cas9 and gRNA are directly delivered via nanoparticles, gene silencing problems will be completely avoided for gene knockouts, and the loading of gRNA can be adjusted to compensate for possible RNA degradation. However, for gene overexpression, traditional strategies (algal host promoters, 3′ and 5′ noncoding regions, and introns) will be used to overcome potential gene silencing.

In some instances, the size of nanoparticle required to package both active Cas9 and the gRNA may be too large to penetrate the cell wall without causing significant damage. To address this possibility, we will pursue two strategies: a one-step nanoparticle delivery of active Cas9 and gRNA and a two-step nanoparticle delivery of a cas9 expression plasmid followed by a gRNA expression plasmid.

We will use aerosol-assisted evaporation-induced self-assembly (see, e.g., Lu Y et al., “Aerosol-assisted self-assembly of mesostructured spherical nanoparticles,” Nature 1999; 398:223-6), a scalable, reproducible technique for producing porous silica nanoparticles of various sizes, to generate nanoparticles for the proposed effort. Our previous studies have indicated that nanoparticle size and surface charge, as well as surface modification with appropriate targeting ligands, are critical to promote effective uptake by mammalian cells and bacteria. Therefore, we will start by synthesizing porous silica nanoparticles with sizes ranging from 20 nm to 200 nm and with surface charges ranging from −30 mV to +30 mV. We can fluorescently label the silica framework of the nanoparticles and to load them with fluorescent surrogates of gRNA and Cas9; we will then employ fluorescence microscopy to track the kinetics of nanoparticle uptake, as well as intracellular dispersion of encapsulated cargo molecules. If necessary, we will modify the silica nanoparticle surface with peptides (e.g. cell-penetrating peptides) and/or proteins (e.g. proteins derived from algal viruses) that are likely to enhance binding and penetration.

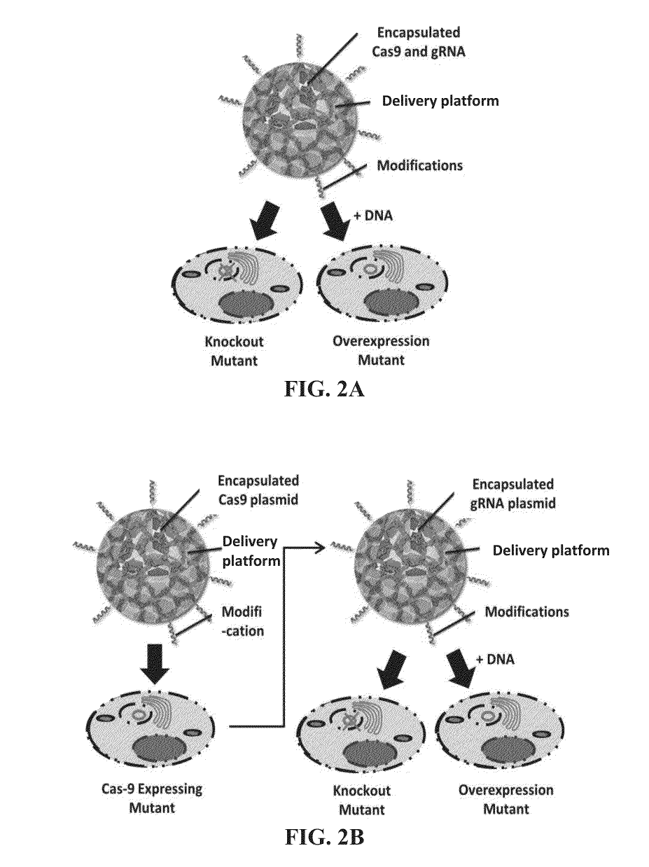

We will pursue two approaches for nanoparticle-mediated CRISPR-Cas9 genome editing: one-step genetic modification and two-step genetic modification (FIG. 2A-2B). In the one-step approach (FIG. 2A), nanoparticles will deliver active Cas9 enzyme, containing a nuclear localization signal, along with the gRNA. If successful, this one-step approach will enable targeted gene knockout without preliminary modification of the host for Cas9 expression. Moreover, transient Cas9 activity in this approach will minimize off-target effects commonly observed with CRISPR-Cas9 technology.

We will also pursue a two-step approach for genetic modification (FIG. 2B). In the two-step approach, a cas9 expression cassette will be randomly integrated into the algal genome via nanoparticle-mediated transformation, and a plasmid containing the gRNA will be delivered in a subsequent step to the Cas9-expressing algal strain. This traditional two-step approach has proven successful in other eukaryotic organisms but requires host expression of both Cas9 and gRNA. To demonstrate utility of the proposed nanoparticle CRISPR-Cas9 technology, both gene knockout and targeted gene insertion will be performed.

### Example 3

### Increasing Algal Production by Limiting Nighttime Loss of Biomass

In order for algal biofuels to become economically feasible, long term area1 production rates need to essentially double. Traditional efforts to improve algal productivities via bioprospecting and algal raceway design have failed to achieve the required improvements leading many to believe that genetic modification may be required. While the model alga, Chlamydomonas reinhardtii, can be easily manipulated, algae with biofuel potential have few tools if any for genetic modification. Therefore, we propose to develop genetic tools for an industrially-relevant alga, Nannochloropsis gaditana. The particle delivery system described herein can be applied for algal genetic transformation. This novel delivery method will be combined with recently-developed, eukaryotic genome editing technology to enable genetic manipulation.

To demonstrate the potential of these methods to improve production, we will target genes involved in dark respiration, a process responsible for a loss of up to 35-67% of carbon fixed during the daylight period (see, e.g., Geider R J, “Respiration: taxation without representation?,” in Primary productivity and biogeochemical cycles in the sea, P. G. Falkowski, Editor 1992, Springer Science & Business Media: New York), thus representing a significant reduction in overall culture productivity.

By studying the energy and organic carbon fluxes of dark respiratory processes in C. reinhardtii and N. gaditana, under a variety of culture conditions, we will identify nonessential pathways contributing to this ‘dark loss’. Utilizing our genetic tools, such pathways will be manipulated to reduce carbon loss and increase overall algal biomass productivities in N. gadiltana.

This approach addresses two critical factors limiting the realization of industrial algal biofuel production: (1) biomass losses resulting from dark respiration and (2) the lack of available tools for genetic manipulation of industrial algae. Dark respiration rates naturally vary by up to two orders of magnitude based on algal species and changes in environmental conditions of temperature, light, as well as CO₂ and O₂ availability (see, e.g., Geider R J et al., “Respiration and microalgal growth: a review of the quantitative relationship between dark respiration and growth,” New Phytologist 1989; 112(3):327-41). This suggests that respiration rates can be rationally manipulated to minimize biomass loss in algal production ponds. Unfortunately, the genetic tools available for manipulation of industrial algal strains are scarce and often inefficient.

Several factors limit algal genetic manipulation: the recalcitrance of algal cell walls which often prevents transformation via conventional methods, the lack of homologous recombination for targeted genome modification, and gene silencing mechanisms (see, e.g., Leon-Bafares R et al., Trends Biotechnol. 2004; 22(1):45-52). The delivery platform described herein (see, e.g., Ashley C E et al., Nat. Mater. 2011; 10(5):389-97; Ashley C E et al., ACS Nano 2012; 6(3):2174-88; and Epler K et al., Adv. Healthc. Mater. 2012 May; 1(3):348-53) can enable algal transformation due to the unique physical and chemical properties.

Additionally, the recent discovery and application of bacterial DNA editing machinery, known as CRISPR/Cas9 technology, has enabled targeted genome editing in eukaryotes which lack the capability for homologous recombination (see, e.g., Sander J D et al., Nat Biotech. 2014; 32(4):347-55). In fact, CRISPR/Cas9 technology has enabled targeted nuclear genome editing in the model alga, Chlamydomonas reinhardtii, illustrating the potential for this technology to enable rational algal optimization. We propose to analyze and manipulate respiratory pathways in an industrially-relevant alga for improved biomass productivity, and to achieve this goal, we will develop nanoparticle-mediated methods for nucleic acid delivery and CRISPR/Cas9 genome editing tools.

Nanoparticles have been demonstrated as not only efficient vehicles for delivery in mammalian and even plant cells, but also as packaging and stabilizing agents for biomolecules such as DNA, RNA, and proteins (see, e.g., Torney F et al., Nat. Nanotechnol. 2007; 2(5):295-300; and Cerutti H et al., “RNA-mediated silencing in algae: biological roles and tools for analysis of gene function,” Eukaryotic Cell 2011; 10(9):1164-72).

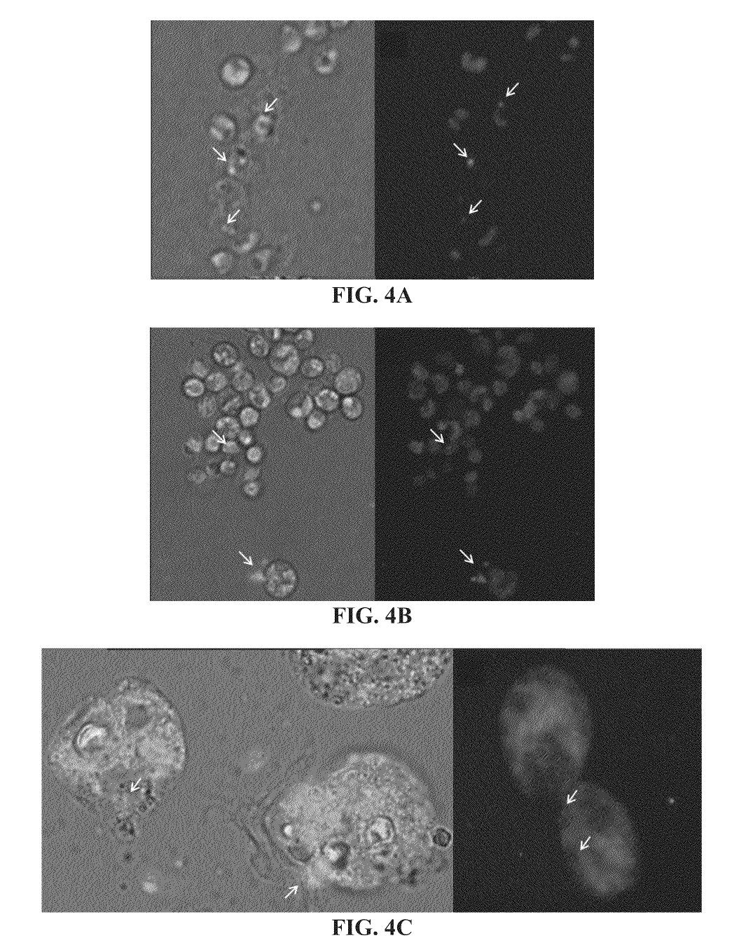

The delivery platform herein can be employed to penetrate through tough algal cell walls, a major obstacle in genetic modification (FIG. 4A-4C). The platform has tunable nanoparticle properties (e.g., particle size, such as of from about 20 nm to about 300 nm in diameter, pore size; chemical composition; lipid composition; lipid charge; and/or surface charge), as well as the possible application of permeabilizing agents (electroporation, particle bombardment, cell penetrating peptides) to promote uptake of the nanoparticles. Screening for nanoparticle uptake can be conducted in a variety of algal species, e.g., C. reinhardtii, P. tricornutum, T. pseudonana, N. gaditana, D. salina, and/or O. tauri.

To develop genetic tools for algal manipulation, we will focus on the industrially-relevant strain: Nannochloropsis gaditana. Not only does N. gaditana have a sequenced genome, but Nannochloropsis species have been touted as prime candidates for industrial production due to their ability to survive under adverse and variable environmental conditions (see, e.g., Jinkerson R E et al., Bioengineered 2013; 4(1):37-43). We will investigate potential obstacles for successful gene expression in N. gaditana by using nanoparticle-mediated delivery of RNA and DNA constructs. In other algal species, degradation of RNA transcripts has been shown to prevent recombinant gene expression (see, e.g., Cerutti H et al., Eukaryotic Cell 2011; 10(9):1164-72). Therefore, RNA constructs of yellow fluorescent protein will be delivered to analyze RNA degradation mechanisms in N. gaditana.

Methods to prevent RNA degradation, including the addition of native 5′ and 3′ non-coding sequences and introns, will be investigated if needed. To develop CRISPR/Cas9 genome editing tools for N. gaditana, the Cas9 nuclease must first be integrated into the genome. To achieve this, we will rely on nanoparticle-mediated delivery and random integration of the recombinant Cas9 DNA fragment with a selectable marker. Transformants will be screened to ensure that random integration of the Cas9 fragment does not affect growth under standard conditions, and Cas9 expression will be confirmed using standard techniques. We will focus on inducible promoters for Cas9 expression to minimize off-target effects of the CRISPR/Cas9 technology (see, e.g., Sander J D et al., Nat Biotech. 2014; 32(4):347-55).

Additionally, nuclear targeting sequences from N. gaditana and other organisms will be investigated to target active Cas9 to the nucleus. For initial demonstration of CRISPR/Cas9 technology in N. gaditana, we will target gene knockout of an amino acid permease in the nuclear genome through design of the guide RNA (gRNA); toxic amino acid analogs can then be used for selection (see, e.g., DiCarlo J E et al., “Genome engineering in Saccharomyces cerevisiae using CRISPR-Cas systems,” Nucleic Acids Res. 2013; 41 (7):4336-43). Gene insertion will also be demonstrated in N. gaditana by including a DNA fragment encoding a yellow fluorescent protein along with the gRNA and Cas9 expression. Demonstration of both gene knockout and gene insertion with CRISPR/Cas9 technology will enable subsequent modification of genetic targets involved in dark loss.

To identify genetic targets for pathway manipulation in N. gaditana, we will leverage the previous work in, and genetic tractability of the model green alga C. reinhardtii (see, e.g., Le Borgne F et al., “Investigation and modeling of biomass decay rate in the dark and its potential influence on net productivity of solar photobioreactors for microalga Chlamydomonas reinhardtii and cyanobacterium Arthrospira platensis,” Bioresource Technol. 2013; 138:271-6). Isothermal, diel conditions will be used to establish transcriptional and metabolic baselines for both species, which we will then perturb by varying the environmental conditions and adding various respiration inhibitors.

Metabolic models can be constructed for both species, which will then be used to identify preliminary targets for genetic modification. The greater genetic tractability of C. reinhardtii will allow us to create and test a variety of modifications. Physiological and transcriptional characterization of the C. reinhardtii mutants will be used to inform the identification of targets for manipulation in N. gaditana. A direct comparison of C. reinhardtii and N. gaditana dark respiration mutants using the respective metabolic models and experimental data will identify common algal dark respiration pathways and nonessential carbon loss mechanisms. Potential differences could arise between the two species due to the evolutionary distance between C. reinhardtii and N. gaditana. Subsequent rounds of metabolic engineering will be conducted in N. gaditana to further optimize dark respiration and overall productivity.

Through rational design and targeted manipulation of an industrially-relevant alga, such as N. gaditana, we will demonstrate that dark respiration losses can be reduced for an overall improvement in biomass productivity. By developing genetic tools of nanoparticle-mediated transformation and CRISPR/Cas9 for N. gaditana, we will enable future genetic engineering efforts to optimize other pathways and traits in this industrial strain. Furthermore, additional algal strains susceptible to nanoparticle-mediated transformation will be identified, expanding the capability for genetic modification of algae well beyond the current list of genetically tractable species.

While it's common knowledge that dark respiration leads to carbon loss (see, e.g., Grobbelaar J U et al., “Respiration losses in planktonic green algae cultivated in raceway ponds,” J. Plankton Res. 1985; 7(4):497-506); and there have been some laboratory studies on the effect of culture conditions on dark loss, such manipulation are not practical in mass culture systems. In addition, there have been no reported efforts of genetic manipulation targeting respiration to improve algal biomass productivity. In fact, most algal genetic engineering efforts to date have focused on increasing tag production and identifying the elusive ‘lipid trigger’ (see, e.g., Sakthivel R S et al., “Microalgae lipid research, past, present: A critical review for biodiesel production in the future,” J. Experiment. Sci. 2011; 2(10):29-49). Traditional studies of dark respiration were conducted in the 1980s and 1990s in algal strains of ecological significance (see, e.g., Geider R I et al., New Phytologist 1989; 112(3):327-41; and Grobbelaar J U et al., J. Plankton Res. 1985; 7(4):497-506). Thus, the manipulation of dark respiration for improved algal biomass productivity remains relatively unexplored. The delivery platform herein provides a versatile genetic tool for enabling successful transformation and manipulation of industrial algae.

### Example 4

### Nanoparticle Uptake in Algae and Potential Genetic Targets

Nanoparticle synthesis: Silica nanoparticles were generated using aerosol-assisted, evaporation-induced, self-assembly (EISA) as previously described (see, e.g., Lu Y et al., Nature 1999; 398:223-6). Briefly, silicates (including DyLight 633-modified silanes) and cetyl trimethylammonium bromide (CTAB) were placed in solution along with ethanol and pH 2 water at room temperature. The resulting homogeneous solution was then atomized into a tubular reactor heated to 450° C. and the resulting powder was collected using filter paper. For samples that did not contain CTAB, CTAB was removed from the silica powder through calcination at 550° C. for 6 hours. Powder samples were then dispersed into DI water at 1 mg/mL and diameter was measured using dynamic light scattering while zeta potential was determined using dynamic electrophoretic mobility. Lipid, polyethylene glycol (PEG), and cholesterol coatings were then added as liposomes to silica particles using previously described methods (see, e.g., Ashley C E et al., Nat. Mater. 2011; 10:389-97). Peptide conjugation was accomplished through the use of a heterobifunctional crosslinker (sulfosuccinimidyl 4-(N-maleimidomethyl)cyclohexane-1-carboxylate) from the surface of either aminated silanes or lipids to peptides modified with C-terminal cysteine groups.

Microalgal Growth:

Both cyanobacteria and eukaryotic microalgae were tested. We have previously shown that cyanobacteria have moderate levels of resistance to ionizing radiation, while literature reports have shown eukaryotic microalgae to be more susceptible to ionizing radiation. The cyanobacterial strains tested include two freshwater species (Synechocystis sp. PCC 6803 and Synechococcus elongatus PCC 7942) and a marine species (Synechococcus sp. PCC 7002). The eukaryotic microalga is a freshwater species, Chlorella sp. NC64A, and two additional eukaryotic algae were tested in the nanoparticle experiments. Growth conditions for each strain are summarized in Table 1. For each culture, 25 mL of media was placed in a 125 mL baffled Erlenmeyer flask, and 1 mL of inoculum was added. Cultures were grown for approximately four days under the aforementioned conditions.

TABLE 1
Growth conditions for cyanobacteria and algae

| | | Temper- | Light | Shak- |
|---|---|---|---|---|
| | | ature | (μmol | ing |
| Strain | Medium | (° C.) | m⁻² s⁻¹) | (rpm) |
| Chlorella sp. NC64A | Modified Bold's | 23 | 100 | 150 |
| | Basal Medium | | | |
| | (MBBM) | | | |
| Haematococcus pluvialis | MES | 23 | 100 | 150 |
| Nannochloropsis salina | F/2 -Si | 23 | 100 | 150 |

Nanoparticle Uptake Experiments:

Three algae strains (Chlorella variabilis NC64A, Haematococcus pluvialis, and Nannochloropsis salina) were grown as described above. During the exponential growth phase, 1 mL of culture was placed in a 1.5 mL microcentrifuge tube with varying amounts of nanoparticles. The tubes were placed on a rocker for continuous mixing and sampled periodically over 24 hours. For sampling, the algae/nanoparticle mixture was centrifuged at 700×g for 10 min, and the pellet is resuspended in 5 μL of supernatant. From this resuspended mixture, 4 μL was added to a glass microscope slide with 0.75% agar. A number 1.0 coverslip was placed on top of the culture and sealed with nail polish. The prepared slide was imaged using IX71 Olympus spinning disk confocal fluorescence microscope. Bright field, GFP fluorescence (Excitation: 472/30 nm, Emission filter: 520/35 nm), and Ch1 fluorescence (Excitation: 480/30 nm, Emission filter: >600 nm) images were acquired. Images were acquired with a IX1 binning and a gain of 296. No neutral density filters were in place. All images were taken at 60× oil with 100 ms exposure and 0.25 μm step sizes. Three to four fields of view were imaged per sample condition. Images were analyzed using Slidebook and ImageJ. Each confocal stack was compressed to a 2D projection over the z axis with optimization of the maximum fluorescence intensity, overlaying the GFP, Ch1, and bright field images to identify cells that had internalized dye-labeled nanoparticles.

Nanoparticle Uptake in Algae:

To enhance delivery of boron and other cargo into microalgae, nanoparticles were investigated as a potential delivery vehicle. The nanoparticles were loaded with Dylight 488, which excites at 493 nm and emits at 518 nm. After mixing the nanoparticles with algal cultures, confocal fluorescence microscopy was used to image the mixture for detection of nanoparticle uptake over time. Three algal strains were tested: Chlorella sp. NC64A, Haemnatococcus pluvialis, and Nannochloropsis salina. These algal species range in size from approximately 1 to 10 p.m. A variety of nanoparticle formulas were tested (Table 2).

TABLE 2
Nanoparticles for intracellular delivery of cargo to algae

| | Avg. | | |
|---|---|---|---|
| Sample | Diameter | Charge | |
| ID | (nm) | (mV) | Surface Coating |
| PF 1.1 | 244 | +27 | CTAB |
| PF 1.2 | 218 | −33 | Hydroxyl Silanes |
| PF 1.3 | 276 | +30 | CTAB, Amino Silanes |
| PF 1.4 | 256 | +33 | Amino Silanes |
| PF 2.1 | 312 | +4 | Amino Silanes, Octa-Arginine Peptide |
| PF 2.2 | 279 | −8 | Zwitterionic Lipid, PEG, Cholesterol |
| PF 2.3 | 309 | −2 | Zwitterionic Lipid, PEG, Cholesterol, |
| | | | Octa-Arginine Peptide |
| PF 2.4 | 284 | −18 | Anionic Lipid (DOPS) |
| PF 2.5 | 247 | +22 | Cationic Lipid (DOTAP) |

Evidence of nanoparticle uptake was detected (FIG. 4A-4C). A summary of the uptake results is included in Table 3.

TABLE 3
Summary of nanoparticle uptake results

| | Conditions showing evidence of NP uptake | | |
|---|---|---|---|
| Particle | Chlorella variabilis | Haematococcus | Nannochloropsis |
| Type | NC64A | pluvialis | salina |
| PF 1.1 | No uptake observed | 1 mg/mL, 24 h | No uptake observed |
| PF 1.2 | No uptake observed | 1 mg/mL, 24 h | No uptake observed |
| PF 2.1 | 1 mg/mL, 21 h | 1 mg/mL, 4, 21, | 100 μg/mL, 4 h |
| | | and 24 h | |
| PF 2.2 | No uptake observed | 1 mg/mL, 24 h | No uptake observed |
| PF 2.3 | 100 μg/mL, 4 h | No uptake observed | 100 μg/mL,4 h |
| PF 2.4 | No uptake observed | No uptake observed | No uptake observed |
| PF 2.5 | No uptake observed | No uptake observed | No uptake observed |

### Example 5

### CRISPR/Cas Targeting in Algae

The present invention relates to use of a lipid-coated silica (LCS) particle for delivering CRISPR/Cas constructs to any useful sample or host cell (e.g., an alga, a plankton, a plant, etc.). In one instance, the alga target is N. gaditana, which is microalga that shows promise for industrial biofuel application due to its capacity to accumulate high levels of fatty acids. Any useful target can be modulated (e.g., activated, inactivated, modified, etc.) by designing a targeting portion (of the guiding component) to have sufficient complementarity to the target sequence.

‘CRISPR’ (Clustered, Regularly-Interspaced, Short Palindromic Repeats) (see, e.g., Barrangou R et al., Science 2007; 315:1709-12) functions as an adaptive immune system for prokaryotes to combat foreign genetic sequences introduced by plasmids and bacteriophages (FIG. 6A-6B). Short segments of foreign nucleic acids derived from plasmids or phage are stored in the microbial CRISPR locus and are used to direct sequence-specific cleavage of foreign genetic elements upon subsequent exposure or infection. Different types of CRISPR systems exist, and each system requires a different number of components. For example, Type II CRISPR systems require only three elements: Cas9 (an endonuclease) and two RNA sequences (i.e., trans-activating CRISPR RNA (or tracrRNA) and CRISPR RNA (or crRNA)). The RNA sequence(s) guide Cas9-mediated cleavage of foreign nucleic acids at specific sequences via base complementarity. In another example, Type I CRISPR systems require at least three elements: a Cascade protein complex, a nuclease (Cas3), and one RNA sequence (crRNA). In another example, Type III CRISPR systems generally require at least two elements: one RNA sequence (crRNA, which is usually further processed at the 3′ end) and a Csm or Cmr complex.

Over the past two years, CRISPR/Cas systems have been used to ‘perform genetic microsurgery’ on mice, rats, bacteria, yeast, plants, and human cells (see, e.g., Mali P et al., Science 2013; 339:823-6; and Zhang F et al., Hum. Mol. Genet. 2014; 23(R1):R40-6). In order to easily manipulate genes using CRISPR, researchers can fuse naturally-occurring tracrRNA and crRNA into a single, synthetic ‘guide RNA’ that directs Cas9 to virtually any desired DNA sequence (see, e.g., FIG. 6C). The synthetic guide RNA includes at least three different portions: a first portion including the tracrRNA sequence, a second portion including the crRNA sequence, and a third portion including a targeting portion or a genomic specific sequence (gRNA) that binds to a desired genomic target sequence (e.g., genomic target DNA sequence, including a portion or a strand thereof). The chimeric tracrRNA-crRNA sequence facilitates binding and recruitment of the endonuclease (e.g., Cas9), and the sgRNA sequence provides site-specificity to the target nucleic acid, thereby allowing Cas9 to selectively introduce site-specific breaks in the target.

These advances have dramatically increased the rate, efficiency, and flexibility with which prokaryotic and eukaryotic genomes can be altered for purposes ranging from basic research to development of therapeutics to manufacture of biofuels. For biodefense or therapeutic applications, CRISPR technology promises to be the foundation for a nimble, flexible capacity to produce medical countermeasures rapidly in the face of any attack or threat via design of guiding components (e.g., guide RNAs) (this can be accomplished rapidly once the genome of target pathogen has been sequenced) that, upon complexation with a Cas enzyme (e.g., Cas9) and intracellular delivery to an infected host cell, cleave target DNA sequences and inhibit pathogen infection.

In vivo applications of CRISPR require a highly efficacious delivery platform. Most nanoparticle delivery platforms have highly interdependent properties, whereby changing one property, such as loading efficiency, affects numerous other properties, such as size, charge, and stability. To address these limitations, we propose a flexible, modular platform for highly efficacious delivery of CRISPR components to plant or alga.

Differentiating features of our approach include: (1) employing CRISPR in place of transient genetic knock-down strategies to reliably and controllably ablate expression of target genes; (2) using lipid coated silica (LCS) technologies (e.g., protocells or silica carriers) to develop a safer, more effective CRISPR delivery platform than current, potentially hazardous lentivirus-based vectors; (3) decoupling the challenge of creating an effective therapeutic from the challenge of creating a therapeutic that, itself, has appropriate adsorption, distribution, metabolism, and excretion; (4) employing CRISPR to solve molecular targeting challenges and leveraging features of our LCS technology to solve macroscopic delivery problems; and (5) using an iterative cycle of predictive modeling, simulation, and experimentation to greatly accelerate the design of efficacious NanoCRISPRs. The synergistic combination of these features will allow us to achieve simultaneous delivery of multiple CRISPR constructs that target multiple different genes in pathogens or host cells.

The CRISPR/Cas system can be implemented to target any useful sequence. The target sequence can include a first nucleic acid that encodes a protein that decreases biomass (e.g., in which case, this protein can be targeted to be down-regulated, such as by cleaving the target sequence with a Cas nuclease) or a protein that increases biomass (e.g., in which case, this protein can be targeted to be up-regulated, such as by activating the target sequence). Then, the guiding component can include a nucleic acid sequence configured to bind to a target sequence of the plant or alga (e.g., configured to bind to the first nucleic acid). In one instance, the guiding component includes a second nucleic acid sequence having sufficient complementarity to the first nucleic sequence, which encodes the protein of interest. In one embodiment, the target sequence includes a first nucleic acid that encodes a protein involved in dark respiration or photorespiration. In some embodiments, the protein is any provided in FIG. 5A-5E. In other embodiments, the guiding component is configured to bind to a target sequence of the plant or alga, in which the target sequence encodes a polypeptide having at least 80% sequence identity to any protein in FIG. 5A-5E (e.g., SEQ ID NOs:201-209) or a fragment thereof.

TABLE 4
Table of potential targets in N. gaditana

| | | | SEQ |
|---|---|---|---|
| | | | ID |
| Pathway | Target Description | Gene Target | NO: |
| Laminarin | Laminarinase | Nga02655 | 201 |
| degradation | | | |
| TAG | TAG lipase CrLIP1 | Nga01367 | 202 |
| degradation | (Chlamydomonas reinhardtii) | | |
| | TGL3/TGL4/TGL5 (yeast) SDP1 | Nga03028 | 203 |
| | (Arabidopsis thaliana) | | |
| Dark | Cytochrome c oxidase COX1 | Nga50029, | 204 |
| respiration | | Nga50030 | 205 |
| | Alternative oxidase AOX1 | Nga03289 | 206 |
| Photo- | Glycolate dehydrogenase | glcD (E. coli) | 207 |
| respiration | Glycolate carboxyligase | glcE (E. coli) | 208 |
| | Tartronic semialdehyde reductase | glcF (E. coli) | 209 |

### Example 6

### Reproducible and Controlled Production of Protocells and Carriers

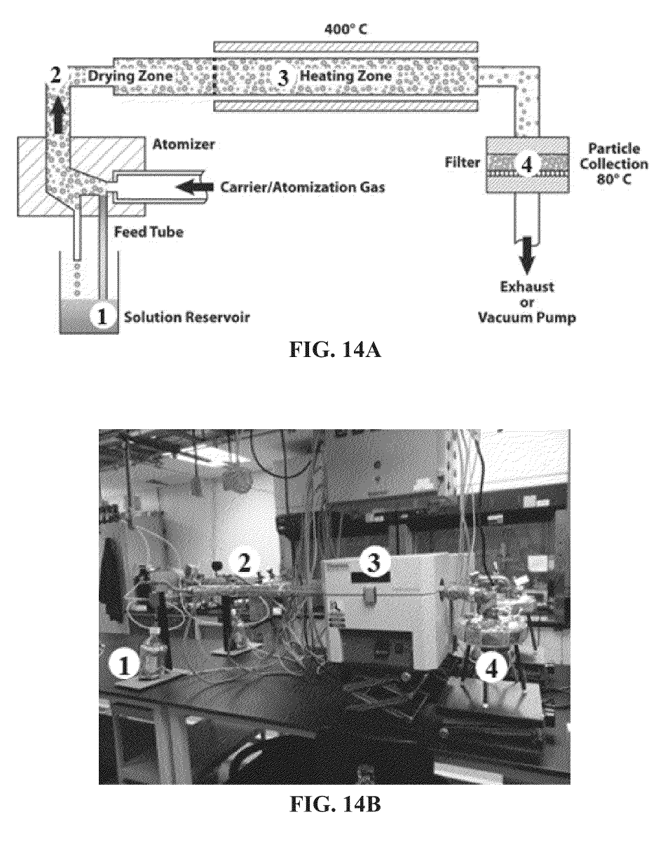

Mesoporous silica nanoparticles (MSNPs) with reproducible properties can be synthesized in a scalable fashion via aerosol-assisted evaporation-induced self-assembly. In the aerosol-assisted EISA process, a dilute solution of a metal salt or metal alkoxide is dissolved in an alcohol/water solvent along with an amphiphilic structure-directing surfactant or block co-polymer; the resulting solution is then aerosolized with a carrier gas and introduced into a laminar flow reactor (FIG. 14A-14B). Solvent evaporation drives a radially-directed self-assembly process to form particles with systematically variable pores sizes (2 to 50 nm), pore geometries (hexagonal, cubic, lamellar, cellular), and surface areas (100 to >1200 m²/g).

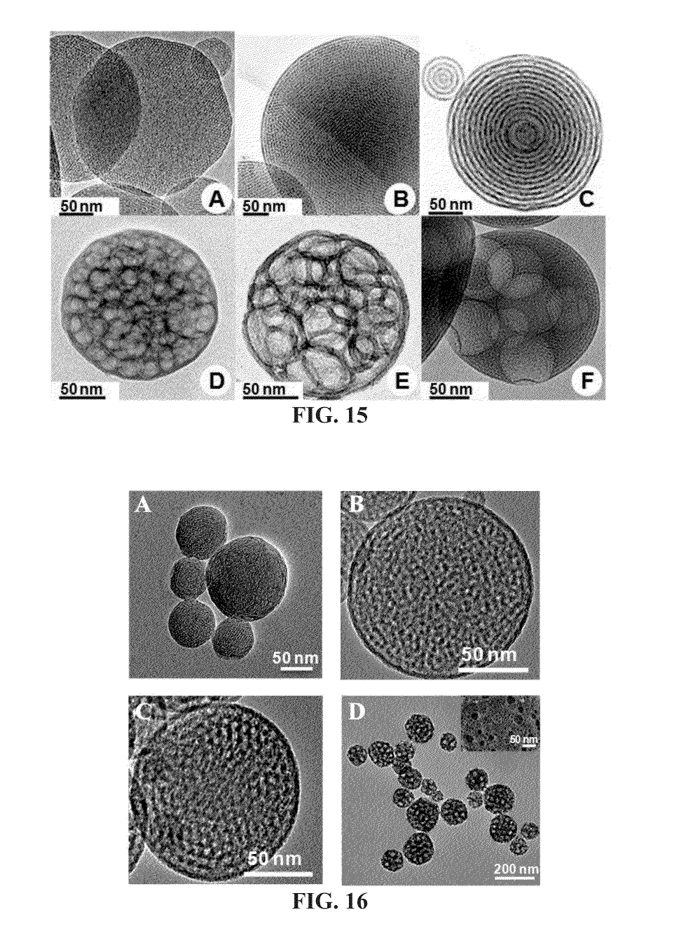

Aerosol-assisted evaporation-induced self-assembly (EISA)(see, e.g., Lu Y F et al., Nature 1999; 398(6724):223-6 and Brinker C J et al., Adv. Mater. 1999; 11(7):579-85) is a robust, scalable process to synthesize spherical, well-ordered oxide nano- and microparticles with a variety of pore geometries and sizes (FIG. 15 and FIG. 16).

Optimization of pore size and chemistry enables high capacity loading of physicochemically disparate biological packages, cargos, or agents, while optimization of silica framework condensation results in tailorable release rates. Despite recent improvements in loading efficiencies and serum stabilities, state-of-the-art liposomes, multilamellar vesicles, and polymeric nanoparticles still suffer from several limitations, including complex processing techniques that are highly sensitive to pH, temperature, ionic strength, presence of organic solvents, lipid or polymer size and composition, and physicochemical properties of the cargo molecule, all of which impact the resulting nanoparticle's size, stability, entrapment efficiency, and release rate (see, e.g., Conley J et al. Antimicrob. Agents Chemother. 1997; 41(6):1288-92; Couvreur P et al., Pharm. Res. 2006; 23(7): 1417-50; Morilla M et al., “Intracellular Bacteria and Protozoa” In Intracellular Delivery, ed. A Prokop, pp. 745-811: Springer Netherlands (2011); and Wong J P et al., J. Controlled Release 2003; 92(3):265-73).

In contrast, particles formed via aerosol-assisted EISA have an extremely high surface area (>1200 m²/g), which enables high concentrations of various therapeutic and diagnostic agents to be adsorbed within the pores of the NP by simple immersion in a solution of the cargo(s) of interest. Furthermore, since aerosol-assisted EISA yields particles that are compatible with a range of post-synthesis modifications, the naturally negatively-charged pore walls can be modified with a variety of functional moieties, enabling facile encapsulation of physicochemically disparate molecules, including acidic, basic, and hydrophobic drugs, proteins, small interfering RNA, DNA oligonucleotides, plasmids, and diagnostic/contrast agents like quantum dots, iron oxide nanoparticles, gadolinium, and indium-111.

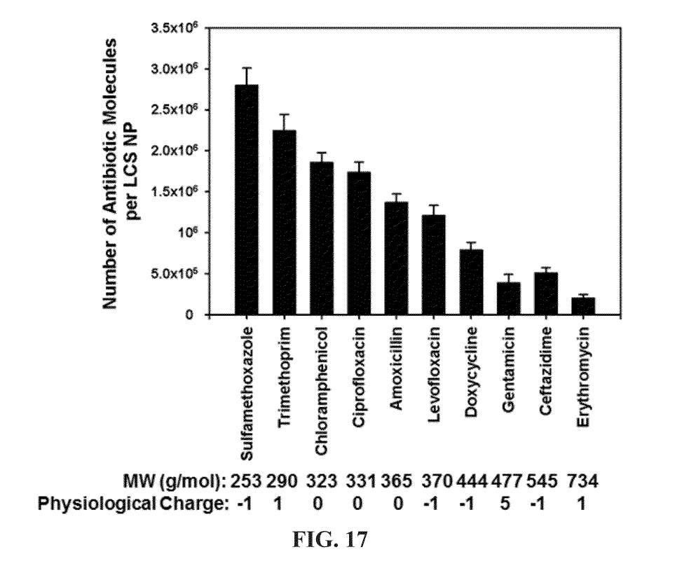

As demonstrated in FIG. 17, particles formed via aerosol-assisted EISA can be loaded with 200,000 to 2,800,000 antibiotic molecules per particle, depending on the molecular weight and net charge of the drug. It is important to note that these capacities are 10-fold higher than other MSNP-based delivery platforms (see, e.g., Clemens D L et al., Antimicrob. Agents Chemother. 2012; 56(5):2535-45) and 100 to 1000-fold higher than similarly-sized liposomes and polymeric nanoparticles (see, e.g., Couvreur P et al., Pharm. Res. 2006; 23(7): 1417-50; Morilla M et al., “Intracellular Bacteria and Protozoa” In Intracellular Delivery, ed. A Prokop, pp. 745-811: Springer Netherlands (2011); and Wong J P et al., J. Controlled Release 2003; 92(3):265-73).

It is also important to note that the particles herein (e.g., protocells or carries) can be loaded with complex combinations of physicochemically disparate agents (e.g., a plurality of small molecule drugs, an antimicrobial peptide, and a phage), a capability other nanoparticle delivery platforms typically do not possess. We are able to achieve high loading capacities for acidic, basic, and hydrophobic drugs, as well as small molecules and macromolecules by altering the solvent used to dissolve the drug prior to loading and by modulating the pore size and chemistry of the particles. Unlike MSNPs formed using solution-based techniques, particles formed via aerosol-assisted EISA are compatible with all aqueous and organic solvents, which ensures that the maximum concentration of drug loaded within the pore network is essentially equivalent to the drug's maximum solubility in its ideal solvent. Furthermore, since particles formed via aerosol-assisted EISA remain stable upon post-synthesis processing, the pore chemistry can be precisely altered by, e.g., soaking naturally negatively-charged particles in amine-containing silanes (e.g., (3-aminopropyl) triethoxysilane, or APTES), in order to maximize electrostatic interactions between pore walls and cargo molecules.

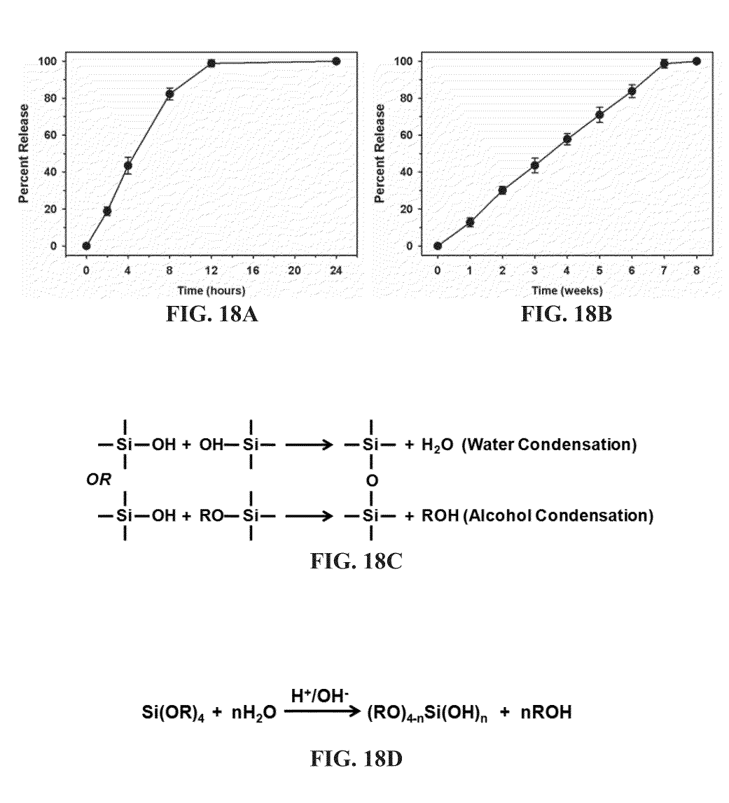

Another unique feature of the delivery platforms herein is that the rate at which encapsulated drugs are released can be precisely modulated by varying the degree of silica framework condensation and, therefore, the rate of its dissolution via hydrolysis under physiological conditions. As shown in FIG. 18A-18D, silica (SiO₂) forms via condensation and dissolves via hydrolysis. Therefore, particles with a low degree of silica condensation have fewer Si—O—Si bonds, hydrolyze more rapidly at physiological pH, and release 100% of encapsulated antibiotics within 12 hours.

In contrast, particles with a high degree of silica condensation hydrolyze slowly at physiological pH and can, therefore, release ˜2% of antibiotics (4,000-56,000 antibiotic molecules per particle, based on the loading capacities shown in FIG. 17) per day for 2 months. We can tailor the degree of silica condensation between these extremes by employing different methods to remove structure-directing surfactants from pores (e.g., thermal calcination, which maximizes the number of Si—O—Si bonds vs. extraction via acidified ethanol, which favors the formation of Si—OH bonds over Si—O—Si bonds) and by adding various concentrations of amine or methyl-containing silanes to the precursor solution in order to replace a controllable fraction of Si—O—Si bonds with Si—R—NH₂ or Si—R—CH₃ bonds, where R=hydrocarbons of various lengths.

### Example 7

### Targeted Delivery Employing the NanoCRISPR Platform

Effective penetration of the NanoCRISPR delivery platform can be promoted in several orthogonal ways. First, the SLB can be optimized with targeting ligands to appropriately bind the target. Second, cell-penetrating peptides can be employed (e.g., associated with the supported lipid bilayer) to facilitate entry. Third, the nanoparticle core can be modified to include a cell penetrating material (e.g., a cell-permeabilizing metal organic framework). Fourth, the LCS delivery platform can be combined with phage technology. All of these strategies can be employed and investigated, in parallel, to provide an effective countermeasure.

Modifying the SLB with targeting ligands promoted efficient uptake of antibiotic-loaded LCS particles by model host cells, which enabled efficient killing of intracellular bacteria. In order to inhibit the intracellular replication of bacteria, nanoparticle delivery platforms must be efficiently internalized by host cells, escape intracellular vesicles, and release encapsulated antibacterials in the host cell's cytoplasm. A number of factors govern cellular uptake and processing of nanoparticles, including their size, shape, surface charge, and degree of hydrophobicity (see, e.g., Peer D et al., Nat. Nanotechnol. 2007; 2(12):751-60). Additionally, a variety of molecules, including peptides, proteins, antibodies, and aptamers, can be employed to trigger active uptake by a plethora of target cells.

We have previously shown that incorporation of targeting and endosomolytic peptides that trigger endocytosis and endosomal escape on the LCS particle SLB enables cell-specific delivery and cytoplasmic dispersion of encapsulated cargos. As importantly, we have shown that SLB fluidity can be tuned to enable exquisite (sub-nanomolar) specific affinities for target cells at extremely low targeting ligand densities (˜6 targeting peptides per LCS particle) and that SLB charge can be modulated to reduce non-specific interactions, resulting in LCS particles that are internalized by target cells 1,000 to 10,000-times more efficiently than non-target cells.

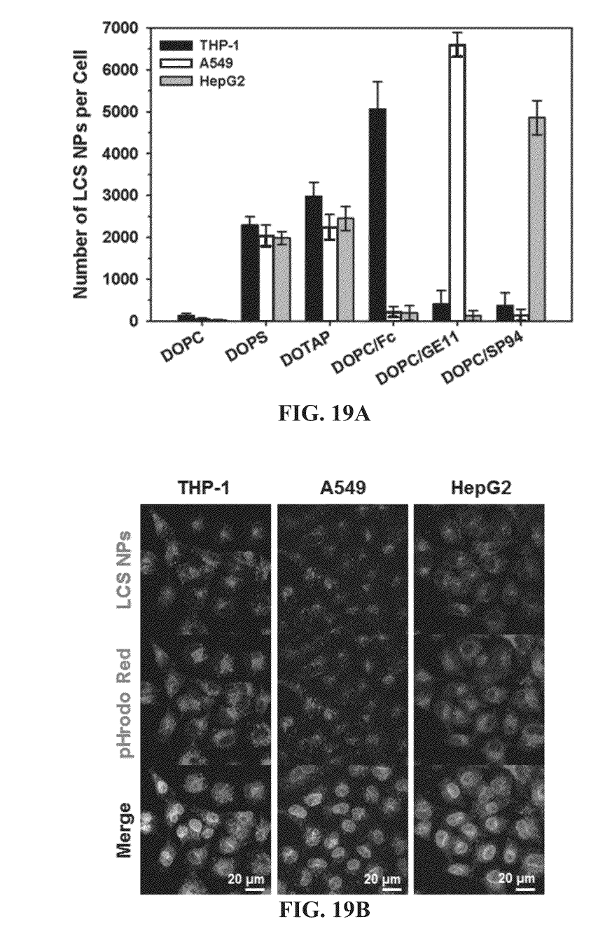

Although originally reported for targeted delivery of chemotherapeutics to cancer, we have utilized the targeting specificity of LCS particles to deliver various antibiotics to host cells in which Bp replicates in vitro. For example, we have shown that modifying DOPC LCS particles with proteins or peptides that target macrophages, alveolar epithelial cells, and hepatocytes triggers a 40 to 200-fold increase in their selective binding and internalization by these cells (FIG. 19A-19B). In contrast, LCS particles with SLBs composed of the anionic lipid, 1,2-dioleoyl-sn-glycero-3-phospho-L-serine (DOPS) or the cationic lipid, 1,2-dioleoyl-3-trimethylammonium-propane (DOTAP) were non-specifically internalized by all cell types, which demonstrates an important point: although numerous researchers use cationic lipids and polymers to coat their NP delivery platforms, the resulting non-specific uptake reduces the effective drug concentration that reaches target cells and tissues (see, e.g., Clemens D L et al., Antimicrob. Agents Chemother. 2012; 56(5):2535-45). In some instances, the LCS particles described herein can be employed to encapsulate and deliver physicochemically disparate cargos or agents (e.g., disparate agents or cargos, including combinations of small molecule, an agrochemical, a carbohydrate, a dye, a marker, a nutrient, a penetrant, a surfactant, a peptide, a protein, a nucleic acid, and/or a phage-based agent).

### Example 8

### Design of the Silica Carrier Platform

In some instances, the biological packages are sufficiently large (e.g., having a dimension greater than about 20 nm), such that deposition within a pore can be difficult. In one non-limiting instance, phage DNA having more than about 10 kpb can have a compacted dimension of about 40 nm. To accomplish effective delivery of such biological packages, the nucleic acid and/or protein can be delivery by way of a silica carrier, in which a thin shell is deposited around the package. The shell can be formed from biocompatible, biodegradable amorphous silica with or without pores.

Therefore, we will adapt our aerosol-assisted EISA process to coat plasmids and phage with amorphous silica shells of varying thicknesses. To do so, we will combine plasmids (5-5000 ng/mL) or phage (10⁶-10⁹ pfu/mL) with a biocompatible silica precursor solution comprised of a water-soluble silica precursor (e.g., tetraethyl orthosilicate [TEOS]), a biocompatible, USP-grade surfactant (e.g., Pluronic® F68, Pluronic® F127, Brij® 58), a plasmid/phage-stabilizing excipient (e.g., sucrose, mannitol, trehalose, polyvinylpyrrolidone, see, e.g., U.S. Pat. No. 6,077,543; Razavi Rohani S S et al., Int'l J. Pharmaceutics 2014; 465(1-2):464-78); and Vehring R. Pharm. Res. 2008; 25(5):999-1022), and a minute amount of HCl to catalyze condensation of silica precursor molecules into silica (see, e.g., Brinker C J, J. Non-crystall. Solids 1988; 100(1-3):31-50)

We will use a double syringe pump and a small-volume mixer to combine plasmids/phage with the silica precursor solution immediately before they are aerosolized using an ultrasonic spray head, an ultrasonic vibrating nebulizer, or a pressurized aerosol generator, resulting liquid droplets will then be fed into a custom-built, laminar-flow reactor using an inert carrier gas (e.g., N₂, which avoids oxidation of plasmids, phage, and excipients) at the inlet and a weak vacuum at the outlet. Droplets will then pass through multiple heating zones with precisely-controlled temperatures that will drive evaporation-induced self-assembly and condensation of amorphous silica shells around plasmids or phage.

To control biodistribution, uptake by the pathogen, and cytoplasmic release of encapsulated phage, we can modulate various properties of the silica carrier, including hydrodynamic size, surface modification with pH-sensitive lipids and targeting ligands, and route of administration. Any useful formulation may be employed, such as spray-dried SPS NPs with excipients to yield an aerosolizable dry powder. The type of excipient and the aerodynamic diameter of the powder can be varied to increase phage shelf-life in the absence of cold chain and to maximize deposition of SPS NPs.

### Example 9

### Design of the Protocell Platform

We have developed scalable strategies for synthesizing highly porous nanomaterials with reproducible properties, thereby providing a way to design the core (e.g., a mesoporous nanoparticle core) of the protocell platform. In this way, the physicochemical properties of the MSNP and SLB can be designed to adapt protocells and related nanoparticle delivery platforms for a wide variety of applications. Here, we describe exemplary design rules to adapt protocells for high capacity loading and controlled release of various countermeasures. We conducted in vitro experiments, and data show that protocells are able to selectively deliver small molecule and nucleic acid-based antivirals to mammalian cells infected with a BSL-2 pseudotype of Nipah virus. Finally, we performed in vivo experiments, which prove that protocells have tailorable biodistributions. These data showed the lack of gross or histopathological toxicity; the presence of ready in vivo degradation and excretion; and the lack of IgG or IgM induction responses, which are indicative of an inflammatory response. These data were observed even when the protocells were modified with high densities of targeting peptides. Additional details follow.

The core of the protocells (e.g., MSNPs) can be prepared with reproducible properties that can be synthesized in a scalable fashion via aerosol-assisted evaporation-induced self-assembly. Aerosol-assisted evaporation-induced self-assembly (EISA) (see, e.g., Lu Y F et al., Nature 1999; 398:223-6) is a robust, scalable process that can be employed to synthesize spherical, well-ordered oxide nano- and microparticles with a variety of pore geometries and sizes. In the aerosol-assisted EISA process, a dilute solution of a metal salt or metal alkoxide is dissolved in an alcohol/water solvent along with an amphiphilic structure-directing surfactant or block co-polymer; the resulting solution is then aerosolized with a carrier gas and introduced into a laminar flow reactor. Solvent evaporation drives a radially-directed self-assembly process to form particles with systematically variable pores sizes (e.g., nanopores, such as those having a size of about 2 nm to 50 nm), pore geometries (e.g., hexagonal, cubic, lamellar, etc.), and surface areas (e.g., 100 to >1,200 m²/g).

Aerosol-assisted EISA, additionally, produces particles compatible with a variety of post-synthesis processing procedures, enabling the hydrodynamic size to be varied from 20 nm to more than 10 μm. Further, pore walls can be modified with a wide range of functional moieties that facilitate high capacity loading of physicochemically disparate diagnostic and/or therapeutic molecules.

Various parameters of the core can be optimized in an independent manner. For instance, optimization of pore size enabled high capacity loading of physicochemically disparate countermeasures, while optimization of silica framework condensation resulted in tailorable release rates. Despite recent improvements in encapsulation efficiencies and serum stabilities, state-of-the-art liposomes, multilamellar vesicles, and polymeric nanoparticles still suffer from several limitations, including complex processing techniques that are highly sensitive to any number of parameters, e.g., pH, temperature, ionic strength, presence of organic solvents, lipid or polymer size and composition, and physicochemical properties of the cargo molecule. All of these parameters impact the resulting nanoparticle's size, stability, entrapment efficiency, and release rate in a non-straightforward manner (see, e.g., Conley J et al., Antinticrob. Agents Chemother. 1997; 41:1288-92; Couvreur P et al., Pharm. Res. 2006; 23:1417-50; Morilla M et al., “Intracellular Bacteria and Protozoa,” In Intracellular Delivery, ed. A Prokop, 2011, pp. 745-811: Springer, Netherlands; and Wong J P et al., J. Controlled Release 2003; 92:265-73). In contrast, MSNPs formed via aerosol-assisted EISA have an extremely high surface area (e.g., more than about 1200 m²/g), which enables high concentrations of various therapeutic and diagnostic agents to be adsorbed within the core by simple immersion in a solution of the cargo(s) of interest.

In particular, for CRISPR components, MSNPs can be synthesized with pores large enough to accommodate Cas9/gRNA components and/or complexes (e.g., any herein). In addition, the MSNPs can be designed to accommodate any other useful cargo, such as entrapped DNA vectors and, if necessary, cell-permeabilizing metal organic frameworks (MOFs) and Bp phage within MSNPs as they are being formed via aerosol-assisted EISA.

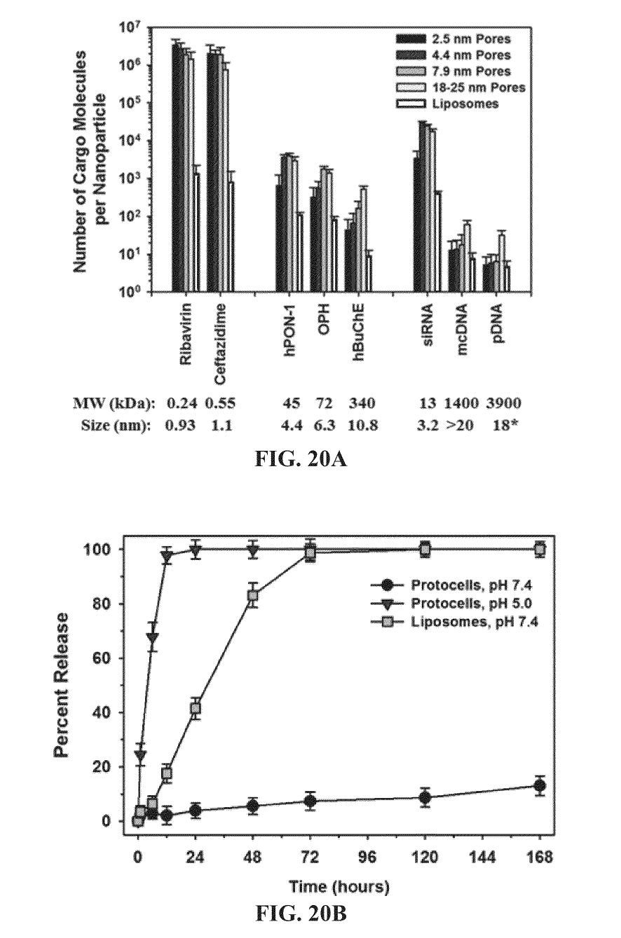

We have previously demonstrated that the loading capacities of MSNPs for various proteins and nucleic acids are maximized when the pore size is slightly larger than the mean hydrodynamic size of the cargo molecule (FIG. 20A). Therefore, in one non-limiting embodiment, MSNPs with pore sizes ranging from 8 nm to 20 nm can be used for encapsulation and delivery of Cas9/gRNA complexes, which have a molecular weight of ˜165 kDa.

Furthermore, since aerosol-assisted EISA yielded MSNPs that are compatible with a range of post-synthesis modifications, the naturally negatively-charged pore walls can be modified with a variety of functional moieties, enabling facile encapsulation of physicochemically disparate molecules, including acidic, basic, and hydrophobic drugs; proteins; small interfering RNA (siRNA); minicircle DNA (mcDNA) vectors that encode small hairpin RNA (shRNA); plasmids (pDNA); and diagnostic/contrast agents like quantum dots, iron oxide nanoparticles, gadolinium, and indium-111 (see. e.g., Ashley C E et al., ACS Nano 2012; 6:2174-88; and Ashley C E et al., Nat. Mater. 2011; 10:389-97).

For instance, NanoCRISPR delivery platforms can include one or more useful surface modifications that promote specific binding and entry of the target. In one instance, NanoCRISPRs can be modified with targeting ligands and endosomolytic ligands to facilitate internalization by model host cells or pathogen cells, as well as endosomal escape and cytosolic dispersion. If needed, BRASIL-based phage display can be employed to identify superior targeting ligands.

As demonstrated by FIG. 20A. MSNPs formed via aerosol-assisted EISA can be loaded with high concentrations of small molecule, protein, and nucleic acid-based countermeasures, and loading capacity is maximized when the pore size is slightly larger than the hydrodynamic size of the cargo molecule. It is important to note that the capacities shown in FIG. 20A are 10-fold higher than other MSNP-based delivery platforms (see, e.g., Clemens D L et al., Antimicrob. Agents Chemother. 2012; 56:2535-45), as well as 100- to 1000-fold higher than similarly-sized liposomes and polymeric nanoparticles (see, e.g., Couvreur P et al., Pharm. Res. 2006; 23:1417-50; Morilla M et al., “Intracellular Bacteria and Protozoa,” In Intracellular Delivery, ed. A Prokop, 2011, pp. 745-811: Springer, Netherlands; and Wong J P et al., J. Controlled Release 2003; 92:265-73). It is also important to note that the MSNPs herein can be loaded with complex combinations of physicochemically disparate countermeasures, a capability other nanoparticle delivery platforms typically do not possess.

Another unique feature of the MSNPs herein is that the rate at which encapsulated agent is released can be precisely modulated by varying the degree of silica framework condensation and, therefore, the rate of its dissolution via hydrolysis under physiological conditions (see, e.g., Ashley C E et al., Nat. Mater. 2011; 10:389-97). The core can be formed from any useful material, such as silica (SiO₂), which forms via condensation and dissolves via hydrolysis. Therefore, MSNPs with a low degree of silica condensation have fewer Si—O—Si bonds, hydrolyze more rapidly at physiological pH, and released 100% of encapsulated drug within 12 hours. In contrast, MSNPs with a high degree of silica condensation hydrolyze slowly at physiological pH and, therefore, released ˜2% of encapsulated drug per day for two months. We can tailor the degree of silica condensation between these extremes by employing different methods to remove structure-directing surfactants from pores (e.g., thermal calcination, which maximizes the number of Si—O—Si bonds versus extraction via acidified ethanol, which favors the formation of Si—OH bonds over Si—O—Si bonds) and by adding various concentrations of amine-containing silanes to the precursor solution in order to replace a controllable fraction of Si—O—Si bonds with Si—R—NH₂ bonds, where R=hydrocarbons of various lengths (e.g., where R is an optionally substituted alkyl, aryl, alkaryl, etc.).

The protocell platform also includes a supported lipid bilayer (SLB). Fusion of liposomes to countermeasure-loaded MSNPs created a coherent SLB that enabled pH-triggered release and provided a biocompatible interface for display of targeting and endosomolytic moieties. Liposomes and multilamellar vesicles have poor intrinsic chemical stability, especially in the presence of serum, which decreases the effective concentration of drug that reaches target cells and increases the potential for systemic toxicity (see, e.g., Couvreur P et al., Pharm. Res. 2006; 23:1417-50; and Morilla M et al., “Intracellular Bacteria and Protozoa,” In Intracellular Delivery, ed. A Prokop, 2011, pp. 745-811: Springer, Netherlands). In contrast, lipid bilayers supported on MSNPs have a high degree of stability in neutral-pH buffers, serum-containing simulated body fluids, and whole blood, regardless of the melting temperature (Tₘ, which controls whether lipids are in a fluid or non-fluid state at physiological temperature) of lipids used to form the SLB (see, e.g., Ashley C E et al., Nat. Mater. 2011; 10:389-97).

Specifically, we have demonstrated that protocells with SLBs composed of the zwitterionic, fluid lipid, 1,2-dioleoyl-sn-glycerol-3-phosphocholine (DOPC) retain small molecule drugs, such as ribavirin, for up to four weeks when incubated in whole blood or a serum-containing simulated body fluid at 37° C. (FIG. 20B). Although protocells are highly stable under neutral pH conditions, the SLB can be selectively destabilized under conditions that simulate the interior volume of intracellular vesicles (e.g., endosomes, lysosomes, and/or macropinosomes), which become acidified via the action of proton pumps. Specifically, DOPC SLBs are destabilized at pH 5.0, which exposed the MSNP core and stimulated its dissolution at a rate dictated by core's degree of silica condensation. Thus, DOPC protocells with MSNPs cores that have a low degree of condensation are, therefore, able to retain ribavirin at pH 7.4 but rapidly release it at pH 5.0 (FIG. 20B).

In order to effectively modify genomic targets in host cells, nanoparticle delivery platforms must be efficiently internalized by host cells, escape intracellular vesicles, and release encapsulated countermeasures in the cytosol of host cells. A number of factors govern cellular uptake and processing of nanoparticles, including their size, shape, surface charge, and degree of hydrophobicity (see, e.g., Peer D et al., Nat. Nanotechnol. 2007; 2:751-60).

Additionally, a variety of molecules, including peptides, proteins, aptamers, and antibodies, can be employed to trigger active uptake by a plethora of specific cells. We have previously shown that incorporation of targeting and endosomolytic peptides that trigger endocytosis and endosomal escape on the protocell SLB enables cell-specific delivery and cytosolic dispersion of encapsulated cargos (see, e.g., Ashley C E et al., Nat. Mater. 2011; 10:389-97). As importantly, we have shown that SLB fluidity can be tuned to enable exquisite (sub-nanomolar) specific affinities for target cells at extremely low targeting ligand densities (˜6 targeting peptides per protocell) and that SLB charge can be modulated to reduce non-specific interactions, resulting in protocells that are internalized by target cells 10,000-times more efficiently than non-target cells. Accordingly, the protocell platform can be designed to accommodate and deliver CRISPR component(s) in an effective and targeted manner.

### Example 10

### Colloidal Stability of Particles

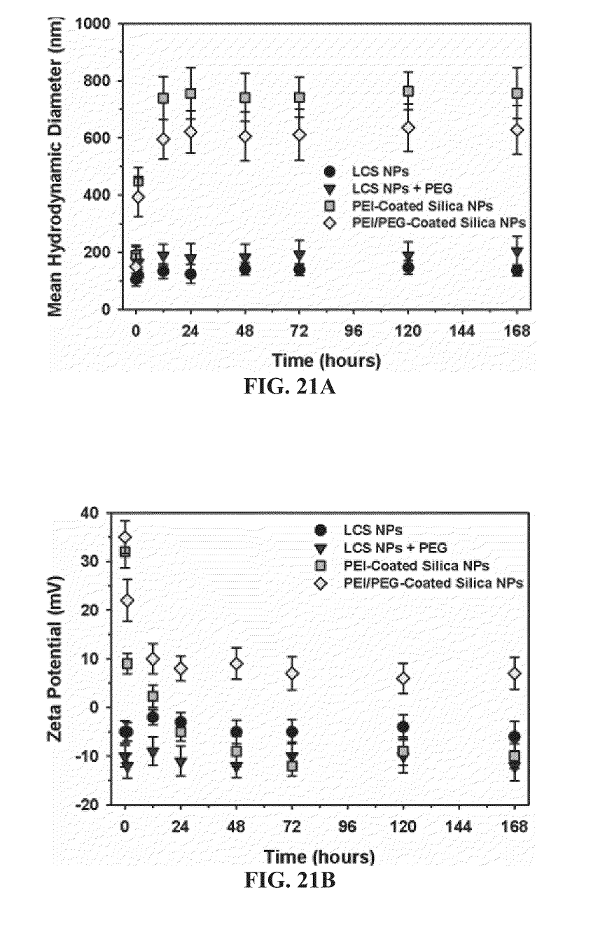

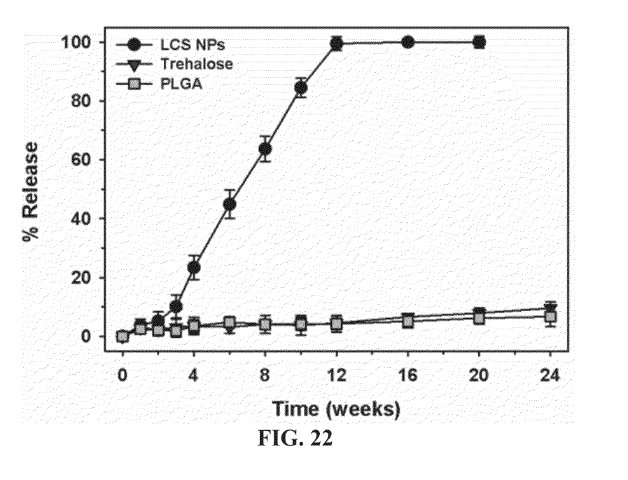

PEG may be a useful ligand to include on a surface of the delivery platform. We have demonstrated that LCS particles with SLBs composed of the zwitterionic, fluid lipid, 1,2-dioleoyl-sn-glycerol-3-phosphocholine (DOPC) have a high degree of colloidal stability (FIG. 21A-21B) in the presence and in the absence of polyethylene glycol (PEG). LCS particles also have longer room-temperature shelf-lives than liposomes or polymeric nanoparticles, the duration of which can be enhanced by spray-drying them in the presence of excipients that protect the lipid shell from drying and thermal stresses and prevent particle aggregation upon re-suspension (FIG. 22).

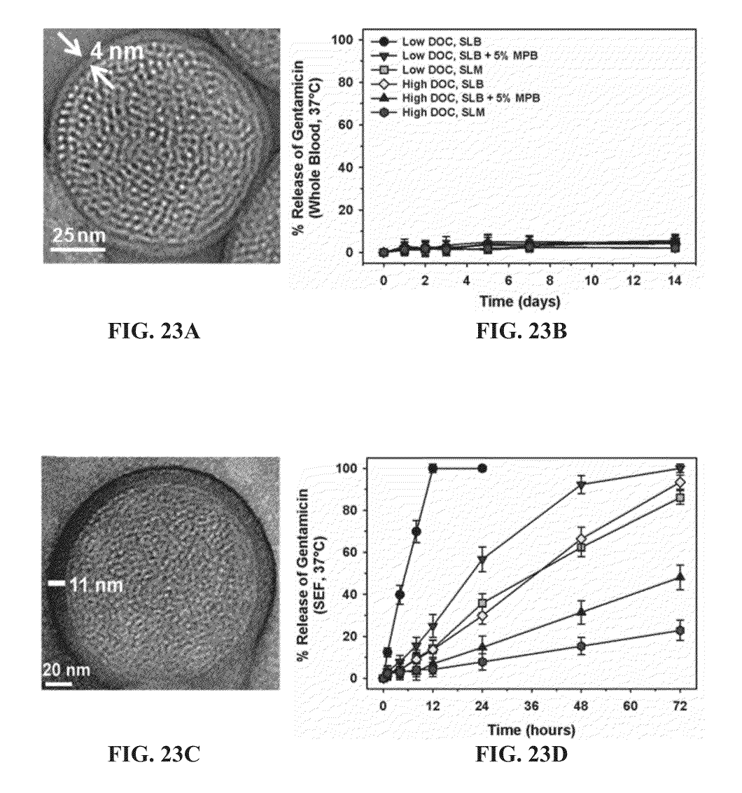

Fusion of liposomes to cargo-loaded particles created a coherent SLB that enhances colloidal stability and enables pH-triggered release. Liposomes and multilamellar vesicles have poor intrinsic chemical stability, especially in the presence of serum, which decreases the effective concentration of drug that reaches target cells and increases the potential for systemic toxicity. In contrast, lipid bilayers supported on particles (see the TEM images in FIG. 23A,23C) have a high degree of stability in neutral-pH buffers, serum-containing simulated body fluids, and whole blood, regardless of the melting temperature (Tₘ, which controls whether lipids are in a fluid or non-fluid state at physiological temperature) of lipids used to form the SLB.

Importantly, LCS particles can be engineered to stably retain encapsulated agents when dispersed in blood (FIG. 23B) but release antibiotics when exposed to conditions that simulate the interior volume of acidic intracellular vesicles, such as endosomes, lysosomes, and phagosomes (FIG. 23D). We have demonstrated that acidic environments destabilize the lipid shell, which exposes the particle core and stimulates its dissolution at a rate dictated by the core's degree of silica condensation. Therefore, by controlling the stability of the lipid shell and the rate at which the particle core dissolves, we can eliminate unwanted leakage of antibiotics in the blood and precisely tailor their intracellular release rates upon uptake of LCS particles by target cells.

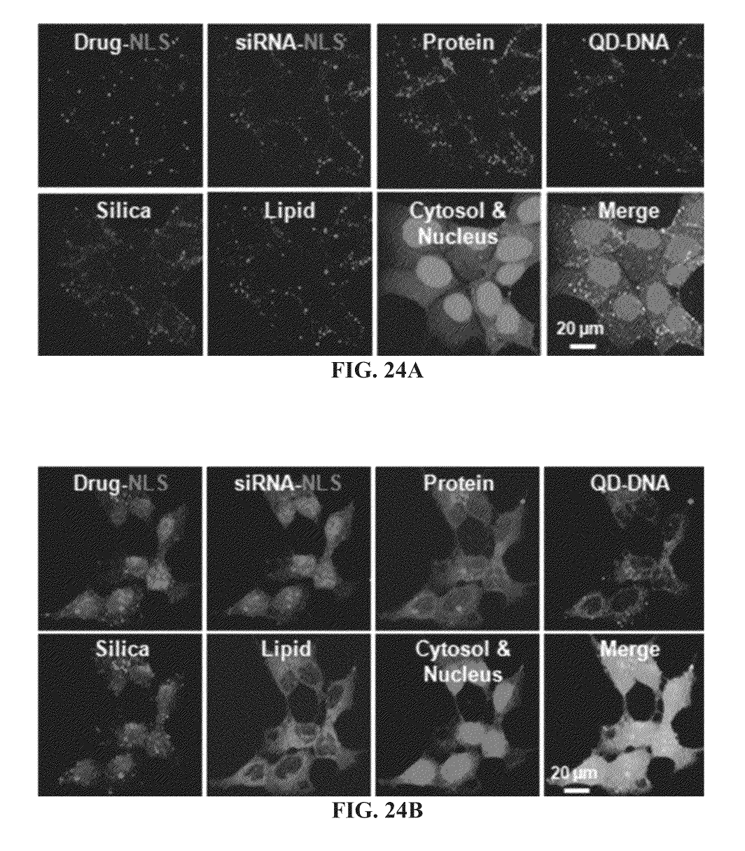

FIG. 24A-24B shows cytoplasmic dispersion of various fluorescently-labeled cargo molecules, as well as the lipid and silica components of protocells. FIG. 24A-24B demonstrates another crucial aspect of our delivery platform technology: unlike liposomes, polymerosomes, and other nanoparticle delivery vehicles, protocells and carriers can simultaneously encapsulate and deliver physicochemically disparate agents in a single platform.

### Example 11

### Biodistribution

For effective in vivo use, any therapeutic agent should be biocompatible. In addition, for targeted uses, biodistribution should be controlled. Generally, these two characteristics can be difficult to control in an independent manner. The platforms herein can be tuned to possess the appropriate biocompatibility and biodistribution based on the associated cargo(s) and/or target.

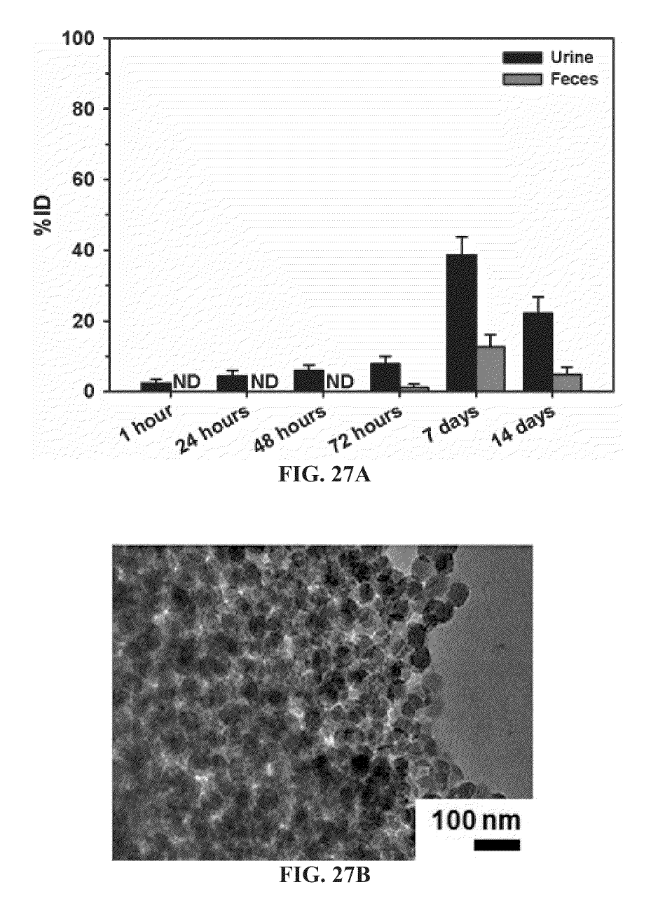

Generally, LCS particles are biocompatible, biodegradable, and non-immunogenic. We have evaluated the biocompatibility, biodegradability, and immunogenicity of LCS particles after repeat intraperitoneal (IP) or subcutaneous (SC) injections in Balb/c and C57B1/6 mice. Balb/c mice injected IP with 200 mg/kg doses of DOPC LCS particles three times each week for four weeks showed no signs of gross or histopathological toxicity. Furthermore, we have demonstrated that intact and partially-degraded particles, as well as silicic acid and other byproducts of silica hydrolysis are excreted in the urine and feces of mice at rates that are determined by the dose, route of administration, and biodistribution (FIG. 27A-27B). These observations that are supported by studies performed previously (see, e.g., Lu J et al., Small 2010; 6:1794-805). Finally, we have shown that LCS particles loaded with a therapeutic protein and modified with a high density (˜10 wt % or 5000 peptides/LCS particle) of a targeting peptide induced neither IgG nor IgM responses upon SC immunization of C57B1/6 mice at a total dose of 1000 mg/kg.

The biodistribution of LCS particles was controlled by tuning their hydrodynamic size and surface modification with targeting ligands. Since liposomes and multilamellar vesicles are the most similar nanoparticle delivery platforms to LCS particles, the performance of LCS particles were benchmarked against the performance of lipid-based nanoparticles. We found that liposomes and multilamellar vesicles, despite being more elastic that LCS particles, can have biodistribution profiles that are largely governed by their overall size and size distributions, an observation that holds true for LCS particles as well. The sizes of liposomes and multilamellar vesicles are, however, difficult to control and subject to slight variations in lipid content, buffer pH and ionic strength, and chemical properties of cargo molecules (see, e.g., Sommerman E F, “Factors influencing the biodistribution of liposomal systems,” Ph.D. dissertation thesis, Dept. of Biochemistry and Molecular Biology, University of British Columbia, 1986, 163 pp.; Comiskey S J et al., Biochemistry 1990; 29:3626-31; and Moon M H et al., J. Chromatogr. A 1998; 813:91-100). In contrast, the diameter of LCS particles was governed by the size of the MSNP core or, in part, by the thickness of the silica shell, which, as we have described herein, is easy to precisely modulate.

The hydrodynamic size of LCS particles dramatically affected their bulk biodistributions: LCS particles (having a diameter of about 250 nm) accumulated in the liver within one hour of injection, while smaller LCS particles (diameter of about 150 nm) remained in circulation for up to two weeks.

Size-dependent biodistribution can be altered, however, by modifying the surface of DOPC LCS particles with various types of targeting ligands. For example, modifying 150 nm LCS particles with CD47, a molecule expressed by erythrocytes that innate immune cells recognize as ‘self’ (see, e.g., Oldenborg P A et al., Science 2000; 288:2051-4), substantially enhanced their circulation half-life. In contrast, modifying 150 nm LCS particles with a proprietary antibody that targets alveolar epithelial cells causes them to rapidly accumulate in the lung. Our ability to engineer LCS particles for high capacity, cell-specific delivery of physicochemically disparate medical countermeasures, as well as our ability to achieve both systemic circulation and targeted accumulation within specific organs demonstrates that LCS particles are an excellent platform on which to base NanoCRISPRs.

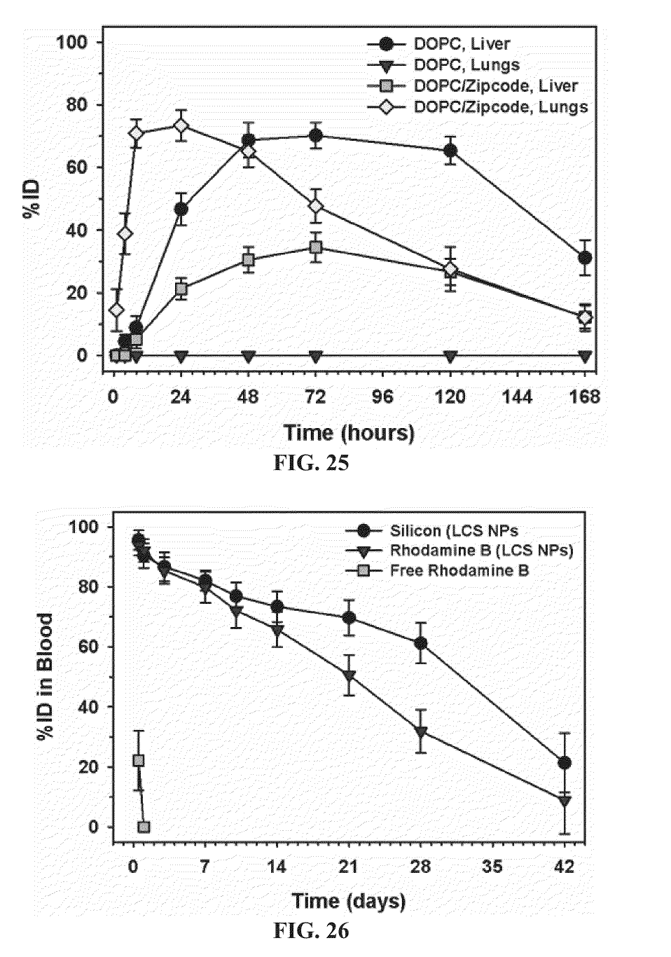

The biodistributions of LCS particles can be controlled by tuning their hydrodynamic diameters, by modifying their surfaces with proteins or peptides that increase circulation times or promote organ-specific accumulation, and by administering them to rodents via parental and non-parental routes. For instance, LCS particles that are 70 nm in diameter also accumulated in the liver and spleen upon IV injection, but their biodistribution can be shifted to favor the lungs by modifying their surfaces with a peptide ‘zip-code’ that binds to lung vasculature (FIG. 25).

Lung accumulation of LCS particles can also be achieved by delivering them as aerosols; LCS particles that are >100 nm in diameter remain in the lung for up to 7 days, while LCS particles that are <100 nm in diameter enter circulation within 8 hours of administration. Finally, LCS particles that are 70 nm in diameter can be engineered to remain in circulation for up to 6 weeks by modifying their surfaces with CD47 (FIG. 26), a protein expressed by erythrocytes that innate immune cells recognize as ‘self’ (see, e.g., Oldenborg P A et al., Science 2000; 288(5473):2051-4). These data demonstrate that LCS particles can be engineered to rapidly accumulate in any useful target host cell.

### Example 12

### Biocompatibility and Biodegradation

Several reasons support our assertion that the amorphous silica that form the cores or shells of LCS particles have low toxicity profiles in vivo: (1) amorphous (i.e., non-crystalline) silica is accepted as ‘Generally Recognized As Safe’ (GRAS) by the U.S. FDA; (2) recently, solid, dye-doped silica nanoparticles received approval from the FDA for targeted molecular imaging of cancer (see, e.g., He Q et al., Small 2009; 5(23):2722-9; and Chen X et al., Acc. Chem. Res. 2011; 44(10):841); (3) compared to solid silica nanoparticles, MSNPs exhibit reduced toxicity and hemolytic activity since their surface porosity decreases the contact area between surface silanol moieties and cell membranes (see, e.g., Tarn D et al., Acc. Chem. Res. 2013; 46(3):792-801; Zhang H et al., J. Am. Chem. Soc. 2012; 134(38):15790-804; and Zhao Y et al., ACS Nano 2011; 5(2):1366-75); (4) the high internal surface area (>1000 m²/g) and ultra-thinness of the pore walls (<2 nm) enable MSNPs to dissolve, and soluble silica (e.g., silicic acid, Si(OH)₄) has extremely low toxicity (see, e.g., He Q et al., Small 2009; 5(23):2722-9; and Lin Y S et al., J. Am. Chem. Soc. 2010; 132(13):4834-42); and (5) in the case of LCS particles, the SLB further reduces interactions between surface silanol moieties and cell membranes and confers immunological behavior comparable to liposomes.

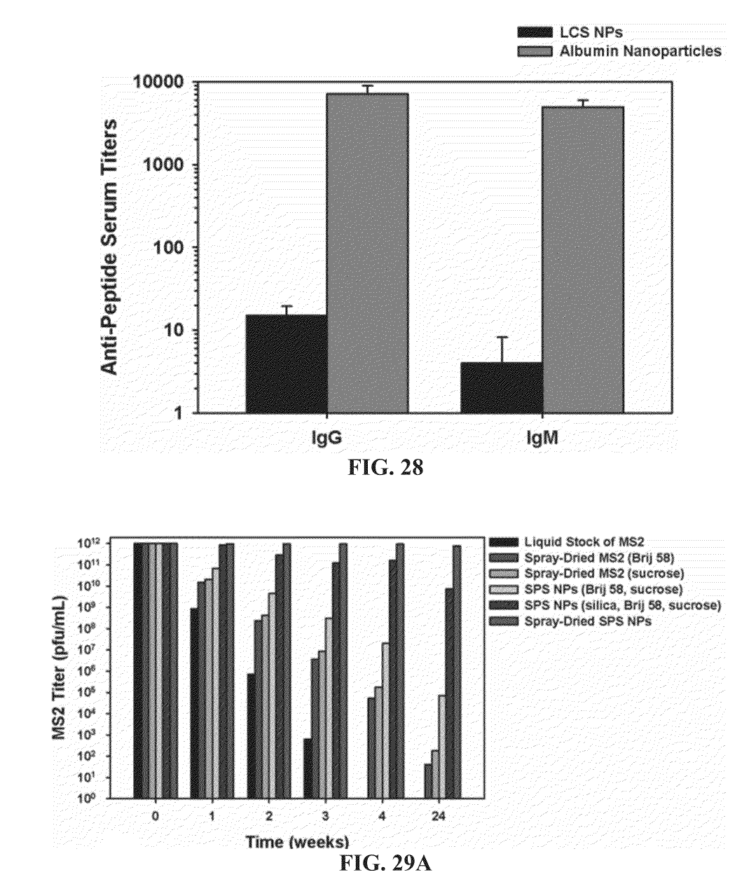

To confirm these observations, we have evaluated the biocompatibility, biodegradability, and immunogenicity of LCS particles after repeat IV or intraperitoneal (IP) injections in mice; BALB/c mice injected IV or IP with large (100 mg/kg) doses of DOPC LCS particles three times each week for 4 weeks showed no signs of gross or histopathological toxicity. Furthermore, we have demonstrated that intact and partially-degraded MSNPs, as well as silicic acid and other byproducts of silica hydrolysis are excreted in the urine and feces of mice at rates that are determined by the dose, route of administration, and biodistribution (FIG. 27A-27B). We have shown that LCS particles modified with a high density (˜10 wt % or ˜5000 peptides per particle) of a targeting peptide induce neither IgG nor IgM responses upon SC immunization of C57BL/6 mice at a total dose of 1000 mg/kg (FIG. 28).

### Example 13

### Spray-Dried Particles and Aerosolized Formulations

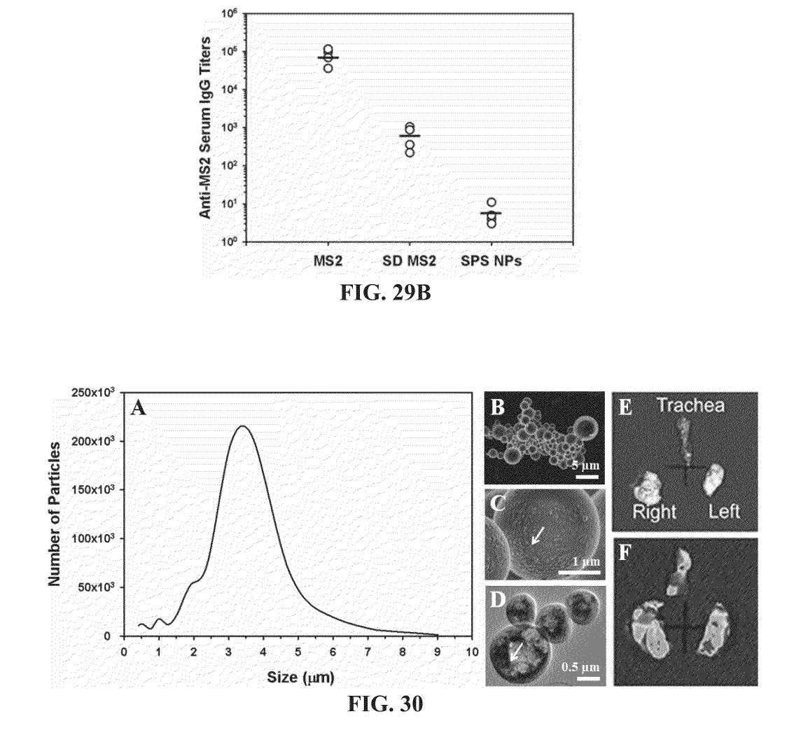

Although spray-drying has been previously used to stabilize phage and adapt them for inhalational administration (see, e.g., Matinkhoo S et al., J. Pharm. Sci. 2011; 100(12):5197-205), aerosol-assisted EISA has several advantages over traditional spray-drying techniques that allow us to precisely control particle size and stability, while maximizing yield and minimizing cost. FIG. 29A shows that carriers (e.g., single phage-in-silica nanoparticles or “SPS NPs”) formed via aerosol-assisted EISA (55 nm mean diameter; one phage per NP, on average) are more stable than spray-dried phage (2.2 μm mean diameter; 42 phage per microparticle, on average).

FIG. 29A also demonstrates the importance of including silica in SPS NP formulations; a model phage (MS2) is ˜16 times more stable upon formulation as SPS NPs that contain silica than upon formulation as SPS NPs that do not contain silica. Furthermore, the silica component of SPS NPs will allow us to precisely control size and release rates, which, in turn, should enable us to tailor biodistribution, maximize phage concentrations at sites of Bp infection, and minimize anti-phage immune responses. As can be seen, SPS NPs dramatically reduced anti-phage antibody responses (FIG. 28), as compared to liquid stock of MS2 or spray-dried MS2 phage.

Furthermore, preliminary experiments indicate that we can generate dry powders that contain 45-57 wt % of SPS NPs and 5.3×10⁹ to 2.8×10¹⁰ pfu/mg of phage (FIG. 30). The powderized form can be aerosolized. For instance, the aerosolized form can include a population of MSNPs, protocells, or silica carriers in a powder form (e.g., prepared with the spray-drying method and the like, or by using a carrier, additive, or excipient and isoniazid, urea, or mixtures thereof that can be administered via the lungs) and including an optional propellant (e.g., a liquefied gas propellant, a compressed gas, or the like).

Aerosol-assisted EISA, additionally, produces particles compatible with a variety of post-synthesis processing procedures, enabling the hydrodynamic size to be varied from 20 nm to >10 μm, and the pore walls to be modified with a wide range of functional moieties that facilitate high capacity loading of physicochemically disparate diagnostic and/or therapeutic molecules. Importantly, aerosol-assisted EISA produces MSNPs that can be easily dispersed in a variety of aqueous and organic solvents without any appreciable aggregation, which enables us to load drugs that have high and low solubility in water.

These particles are also easily encapsulated within anionic, cationic, and zwitterionic supported lipid bilayers (SLBs) via simple liposome fusion. In contrast, particles generated using solution-based techniques aggregate when the pH or ionic strength of their suspension media changes (see, e.g., Liong M et al., J. Mater. Chem. 2009; 19(35):6251-7), typically require complex strategies involving toxic solvents to form SLBs, and have maximum loading capacities of 1-5 wt %, which our MSNPs exceed by an order of magnitude (see, e.g., Cauda V et al., Nano Lett. 2010; 10(7):2484-92; Schlolbauer A et al., Adv. Healthc. Mater. 2012; 1(3):316-20; and Clemens D L et al., Antimicrob. Agents Chemother. 2012; 56(5): 2535-45).

### OTHER EMBODIMENTS

All publications, patents, and patent applications, including U.S. Provisional Application No. 62/057,968, filed Sep. 30, 2014, and U.S. Provisional Application No. 62/129,028, filed Mar. 5, 2015, mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

1. A delivery platform for transforming a plant or an alga, the platform comprising:
a biological package comprising:
a guiding component configured to bind to a target sequence of the plant or alga, or a nucleic acid encoding the guiding component; and/or
a nuclease or a nucleic acid encoding the nuclease, wherein the nuclease is configured to interact with the target sequence after binding by the guiding component;
a particle configured to contain the biological package, wherein the particle comprises an outer surface; and
a supported lipid layer disposed on the outer surface of the particle.

2. The platform of claim 1, wherein the particle comprises a porous core comprising a plurality of pores, and wherein the biological package is disposed within at least one pore.

3. The platform of claim 2, wherein the porous core comprises a metal oxide.

4. The platform of claim 2, wherein the porous core comprises a mesoporous nanoparticle.

5. The platform of claim 4, wherein the porous core is spherical and ranges in diameter from about 10 nm to about 250 nm.

6. The platform of claim 1, wherein the particle comprises an encapsulating shell configured to encapsulate the biological package.

7. The platform of claim 6, wherein the shell has a thickness of from about 0.1 nm to about 10 nm.

8. The platform of claim 7, wherein the shell comprises a metal oxide.

9. The platform of claim 7, wherein the shell comprises an amorphous silica.

10. The platform of claim 7, wherein the shell is porous or non-porous.

11. The platform of claim 1, wherein the biological package comprises a plasmid.

12. The platform of claim 11, wherein the plasmid encodes the guiding component and/or the nuclease.

13. The platform of claim 1, wherein the nuclease is configured to bind the target sequence and/or cleave the target sequence.

14. The platform of claim 1, wherein the guiding component comprises:
a targeting portion comprising a nucleic acid sequence configured to bind to the target sequence; and
an interacting portion comprising a nucleic acid sequence configured to interact with the nuclease.

15. The platform of claim 14, wherein the target sequence encodes a polypeptide having at least 80% sequence identity to any one of SEQ ID NOs:201-209, or a fragment thereof.

16. The platform of claim 14, wherein the target sequence encodes a polypeptide selected from the group consisting of a lipase, a laminarinase, an oxidase, a dehydrogenase, a ligase, and a reductase, or a fragment thereof.

17. The platform of claim 14, wherein the interacting portion comprises a structure:
A-L-B,
wherein:
A comprises a nucleic acid sequence having at least 80% sequence identity to any one of SEQ ID NOs:20-32 and 70 or a complement of any of these, or a fragment thereof,
L is a linker, and
B comprises a nucleic acid sequence having at least 80% sequence identity to any one of SEQ ID NOs:40-54, 60-65, and 71 or a complement of any of these, or a fragment thereof.

18. The platform of claim 17, wherein the interacting portion comprises a nucleic acid sequence having at least 80% sequence identity to any one of SEQ ID NOs:80-93 and 100-103 or a complement of any of these, or a fragment thereof.

19. The platform of claim 1, wherein the nuclease is a Cas protein, a modified form thereof, or a deactivated form thereof.

20. The platform of claim 19, wherein the Cas protein comprises an amino acid sequence having at least 80% sequence identity to any one of SEQ ID NOs: 110-117, or a fragment thereof.

21. The platform of claim 1, further comprising an additional cargo disposed within the particle, on a surface of the particle, in proximity to the biological package, and/or within a pore of the particle.

22. The platform of claim 21, wherein the additional cargo comprises a nucleic acid, a polypeptide, a small molecule, an agrochemical, a carbohydrate, a dye, a marker, a nutrient, a penetrant, and/or a surfactant.

23. The platform of claim 1, wherein the supported lipid layer further comprises one or more targeting ligands having an amino acid sequence having at least 80% sequence identity to any one of SEQ ID NOs: 1-6, 210-238, and 240-249, or a fragment thereof.

24. An aerosolized formulation comprising a plurality of delivery platforms of claim 1 and a propellant.

25. The formulation of claim 24, wherein the mean particle size is of from about 2 to about 5 μm.

26. A liquid formulation comprising a plurality of delivery platforms of claim 1 and an aqueous solution.

27. A powdered formulation comprising a plurality of delivery platforms of claim 1 and an optional excipient.

28. A transformed plant or alga comprising a delivery platform of claim 1.

29. A method of transforming a plant or an alga, the method comprising:
administering a delivery platform of claim 1 to the plant or alga, thereby modulating the target sequence of the plant or alga.

30. The method of claim 29, further comprising:
fermenting and/or liquefying the plant or alga, thereby obtaining a biomass;
extracting one or more lipids from the biomass, or a fraction thereof; and
optionally processing the one or more lipids, or a fraction thereof, to form a biofuel.
